SEQUENCE LISTING (28 sequences)

SEQ ID NO: 1 - DNA, 97 bp, Human
tgctgttgacagtgagcgcaggaagaattgttttaggatatagtgaagccacagatgtatatcctaaaacaattcttcctttgcctactgcctcgga

SEQ ID NO: 2 - DNA, 97 bp, Human
tgctgttgacagtgagcgaaggcagaagtatgcaaagcattagtgaagccacagatgtaatgctttgcatacttctgcctgtgcctactgcctcgga

SEQ ID NO: 3 - DNA, 21 bp, Human
ttccacgatatggatttacgg

SEQ ID NO: 4 - DNA, 21 bp, Human
gtttcctgttgcattgagtcc

SEQ ID NO: 5 - DNA, 20 bp, Human
ccctcctcaggtggtgtttg

SEQ ID NO: 6 - DNA, 20 bp, Human
cgacaaatgaagcagcgtgt

SEQ ID NO: 7 - DNA, 21 bp, Human
gacgcctttatcctctctgcg

SEQ ID NO: 8 - DNA, 21 bp, Human
acgacctgatccaattctgcc

SEQ ID NO: 9 - DNA, 21 bp, Human
ctgaccgcagagtcttttcct

SEQ ID NO: 10 - DNA, 20 bp, Human
gagtggtttggctggggtaa

SEQ ID NO: 11 - DNA, 21 bp, Human
ctgcctgggtttccatcttct

SEQ ID NO: 12 - DNA, 23 bp, Human
gccattcatgtcagagctacact

SEQ ID NO: 13 - DNA, 21 bp, Human
ctcgccagtgaaatgatggct

SEQ ID NO: 14 - DNA, 21 bp, Human
gtcggagattcgtagctggat

SEQ ID NO: 15 - DNA, 20 bp, Human
cgcccgaagacatctaccag

SEQ ID NO: 16 - DNA, 21 bp, Human
gatattccaagagcgtgtgcc

SEQ ID NO: 17 - DNA, 26 bp, Human
gctaacctttgatgctataactacga

SEQ ID NO: 18 - DNA, 21 bp, Human
tttgtgcgcatgtagaatctg

SEQ ID NO: 19 - DNA, 22 bp, Human
catcaatgacaaggtggccaag

SEQ ID NO: 20 - DNA, 20 bp, Human
gggcttgatggggtgatagg

SEQ ID NO: 21 - DNA, 19 bp, Human
gatgggcggcggaaaatag

SEQ ID NO: 22 - DNA, 21 bp, Human
gcgtggattctgcataatggt

SEQ ID NO: 23 - DNA, 20 bp, Human
acccccatgactccagagaa

SEQ ID NO: 24 - DNA, 21 bp, Human
cttgtgcctgcaaaatcgcat

SEQ ID NO: 25 - DNA, 22 bp, Human
caccctctagcaatgtcaactc

SEQ ID NO: 26 - DNA, 21 bp, Human
cagactcgggatactctctcc

SEQ ID NO: 27 - DNA, 20 bp, Human
ggagcgcaccatcttcttca

SEQ ID NO: 28 - DNA, 20 bp, Human
gggccgcatcttcacctacc

(19) United States
(12) Patent Application Publication
(10) Pub. No.: US 2023/0032216 A1
(43) Pub. Date: Feb. 2, 2023

(54) INTERLEUKIN-4-INDUCED GENE 1 (IL4I1) AND RESPECTIVE METABOLITES AS BIOMARKERS FOR CANCER

(71) Applicant: DEUTSCHES KREBSFORSCHUNGSZENTRUM STIFTUNG DES ÖFFENTLICHEN RECHTS, Heidelberg (DE)

(72) Inventors: Alexander Boehme, Leipzig (DE); Saskia Trump, Leipzig (DE); Luis Felipe Somarribas Patterson, Heidelberg (DE); Ahmed Sadik, Heidelberg (DE); Christiane A. Opitz, Heidelberg (DE)

(73) Assignee: Deutsches Krebsforschungszentrum Stiftung des oeffentlichen Rechts Im Neuenheimer Feld 280, Heidelberg (DE)

(21) Appl. No.: 17/783,953

(22) PCT Filed: Dec. 10, 2020
(86) PCT No.: PCT/EP2020/085647

§ 371 Date: Jun. 9, 2022

(30) Foreign Application Priority Data
Dec. 10, 2019 (EP) 19214890.6

(51) Int. Cl. C12Q 1/6886

(52) U.S. Cl. CPC C12Q 1/6886; C12Q 2600/158

(57) ABSTRACT

The present invention relates to the use of Interleukin-4-induced gene 1 (IL4I1) as well as metabolites as produced by IL4I1 as marker in diagnosis and therapy of cancer and related metastasis and/or resistance to immunotherapy.

The present invention relates to the use of Interleukin-4-induced gene 1 (IL4I1) as well as metabolites as produced by IL4I1 as markers in diagnosis and therapy of cancer and related metastasis and/or resistance to immunotherapy.

### BACKGROUND OF THE INVENTION

IL4I1 is an L-amino acid oxidase that catalyzes the oxidative deamination of L-amino acids to alpha-keto acids while producing hydrogen peroxide and ammonia. Initially discovered as an immediate-early IL4-inducible gene in B cells, IL4I1 was later on also identified in macrophages and dendritic cells (Molinier-Frenkel et al., 2019).

In cancer patients, IL4I1 is expressed either by tumor cells themselves (e.g., some B cell lymphoma subsets, mesothelioma or ovarian cancer and gliomas) or by tumor-associated macrophages (TAM) or dendritic cells (DC) (Carbonnelle-Puscian, et al. 2009). It is a secreted enzyme physiologically produced by antigen presenting cells (APC) of the myeloid and B cell lineages and T helper type (Th) 17 cells (Molinier-Frenkel et al., 2019). Important roles of IL4I1 in the fine control of the adaptive immune response in mice and humans have emerged during the last few years. Indeed, IL4I1 inhibits T cell proliferation (Lasoudris et al., 2011) and cytokine production and facilitates naive CD4+ T-cell differentiation into regulatory T cells in vitro by limiting the capacity of T lymphocytes to respond to clonal receptor stimulation. It may also play a role in controlling the germinal center reaction for antibody production and limiting Th1 and Th17 responses (Molinier-Frenkel et al., 2019).

Currently, only very little is known about the therapeutic implications of IL4I1-associated conditions. IL4I1 is expressed in tumor-associated macrophages of most human cancers and in some tumor cell types. Such expression, associated with its capacity to facilitate tumor growth by inhibiting the anti-tumor T-cell response, makes IL4I1 a new potential target in the field of immunomodulation in cancer.

Some compounds with alleged IL4I1 inhibitory activity were disclosed in WO 2010/066858. However, the specifically disclosed compounds were found either toxic or with unsatisfactory IL4I1 inhibitory activity. The disclosed compounds had Ki in the mM range, thus lacking efficacy, and rapidly induced complete cell death of a T cell line (Jurkat cells).

WO 2016/040488 discloses methods of promoting myelin formation in central nervous system (CNS) tissue in a subject in need thereof, the method comprising administering to the subject a therapeutically effective amount of IL4I1 protein.

WO 2019/185907 relates to the inhibition of IL4I1, found to be expressed in a large set of human cancers. Provided are compounds for a use in the treatment of cancer that are phenylalanine-derivatives according to a given formula (I). The cancer to be treated displays IL4I1-expressing cells.

There is also very limited information about diagnostic uses of IL4I1 and its metabolic functions in the context of cancer, in particular metastatic cancer.

Lasoudris et al. (in: IL4I1: an inhibitor of the CD8⁺ antitumor T-cell response in vivo, Eur J Immunol. 2011 June;41(6):1629-38) disclose that the L-phenylalanine oxidase IL4I1 inhibits T-cell proliferation in vitro through hydrogen peroxide (H₂O₂) production, and is highly expressed in tumor-associated macrophages. Immunosuppressive functions of IL4I1 are demonstrated in vivo and suggest that IL4I1 facilitates human tumor growth by inhibiting the CD8(+) antitumor T-cell response.

IL4I1 has also been disclosed as part of a so-called stroma-derived prognostic predictor (SDPP) that stratifies disease outcome in breast cancer, renal cancer, and glioma (Finak et al., Stromal gene expression predicts clinical outcome in breast cancer. Nat. Med. 2008; 14:518-527).

Molinier-Frenkel et al. (The IL4I1 Enzyme: A New Player in the Immunosuppressive Tumor Microenvironment. Cells. 2019 Jul. 20;8(7)) disclose that as a secreted enzyme, IL4I1 may represent an easily targetable molecule for cancer immunotherapy.

Bod L., et al. (IL4-induced gene 1 promotes tumor growth by shaping the immune microenvironment in melanoma. Oncoimmunology. 2017;6:e1278331) showed that IL4I1 activity correlated with disease aggressiveness, although they found that IL4I1 did not enhance tumor cell proliferation or angiogenesis.

Cheong and Sun (Targeting the IDO1/TDO2-KYN-AhR Pathway for Cancer Immunotherapy—Challenges and Opportunities. Trends Pharmacol Sci. 2018 Mar;39(3):307-325) review recent progress and future perspectives in targeting the IDO1/TDO2-KYN-AhR signaling pathway for the development of novel cancer immunotherapies.

So far, the secreted IL4I1 (interleukin-4-induced 1) enzyme was disclosed as catabolizing L-phenylalanine, and to a lesser extent arginine to generate H₂O₂, ammonia (NH₃) and the corresponding α-keto acid (Molinier-Frenkel et al., 2019; Boulland et al., 2007; Mason et al., 2004). However, it was found that IL4I1 is the key enzyme of a newly identified Trp-catabolic pathway that yields AHR agonists, including indole metabolites. IL4I1 associates with reduced survival in glioma patients, promotes cancer cell motility, and inhibits T-cell proliferation. Being a more potent AHR activator than IDO1, IL4I1 may explain the failure of clinical studies combining immune checkpoint blockade (ICB) with IDO1 inhibitors: ICB induces IL4I1, which mediates therapy escape. Thus, IL4I1 blockade will open new avenues for cancer therapy. From these findings it is clearly inferred that IL4I1 based treatments provide new avenues for resistance to immunotherapy, conventional therapies leading to immune activation and therapies targeting or inhibiting AHR activation (e.g. IDO/TDO2 inhibitors).

An object of the invention is therefore to identify new approaches to use IL4I1 and its metabolic functions in methods for diagnosing and monitoring cancer. Other objects and aspects will become apparent when studying the following more detailed description of the invention.

The aryl hydrocarbon receptor (AHR) is a ligand-activated transcription factor that enables cells to adapt to changing conditions by sensing compounds from the environment, diet, microbiome, and cellular metabolism (Rothhammer and Quintana, 2019). Initially discovered as the mediator of the toxic effects of the xenobiotic 2,3,7,8-tetrachlorodibenzo-p-dioxin (TCDD, dioxin), the AHR was subsequently shown to exert important roles in development, immunity, and cancer (Boitano et al., 2010; Fernandez-Salguero et al., 1995; Gutierrez-Vazquez and Quintana, 2018; Kiss et al., 2011; Murray et al., 2014; Nguyen and Bradfield, 2008; Rentas et al., 2016; Stockinger et al., 2014). Upon ligand binding the AHR translocates from the cytoplasm into the nucleus, where it heterodimerizes with the AHR nuclear translocator (ARNT) and induces transcription by binding to xenobiotic response elements (XRE) (Nguyen and Bradfield, 2008). AHR target genes regulate a variety of biological processes, including angiogenesis, hematopoiesis, drug and lipid metabolism, cell motility, and immune modulation (Denison and Nagy, 2003; Gutierrez-Vazquez and Quintana, 2018; Murray et al., 2014; Nguyen and Bradfield, 2008; Stockinger et al., 2014). Next to xenobiotics, natural ligands derived from plants, microbiota and endogenous metabolism are potent AHR agonists (Denison and Nagy, 2003; Li et al., 2011; Rothhammer and Quintana, 2019). Tryptophan (Trp) derivatives constitute an important class of endogenous AHR ligands (Denison and Nagy, 2003; Hubbard et al., 2015; Nguyen and Bradfield, 2008). The kynurenine (Kyn) pathway, initiated by indoleamine-2,3-dioxygenase ½ (IDO½) or tryptophan-2,3-dioxgenase (TDO2), is currently considered as the main Trp catabolic route in humans (Cervenka et al., 2017; Lemos et al., 2019; Platten et al., 2019), yielding the AHR agonists Kyn and kynurenic acid (KynA) (DiNatale et al., 2010; Mezrich et al., 2010; Opitz et al., 2011). Cancers express high levels of IDO1 and TDO2, and take advantage of the effects of Trp catabolite-mediated AHR activation to enhance their malignancy and to inhibit anti-tumor immune responses.

The Kyn-AHR axis suppresses T cell proliferation and function by inducing (i) the differentiation of regulatory T cells (Tregs) (Mezrich et al., 2010; Quintana et al., 2010), (ii) the expression of the immune checkpoint molecule programmed cell death protein 1 (PD-1) on CD8⁺ T cells (Liu et al., 2018), (iii) cell death of CD8⁺ T cells (Greene et al., 2019), and (iv) the recruitment of immunosuppressive tumor-associated macrophages (TAM) (Takenaka et al., 2019). Next to immune effects, the Kyn-AHR axis also enhances the malignant phenotype of cancer cells, the most prominent of which is cancer cell motility (Chen et al., 2014; D'Amato et al., 2015; Novikov et al., 2016; Opitz et al., 2011; Xiang et al., 2019).

Inhibition of Trp-catabolizing enzymes (TCEs) thus represents a promising strategy to target both cancer cell malignancy and tumor-derived immunosuppression (Lemos et al., 2019; Platten et al., 2019).

Small molecule inhibitors of IDO1 have entered clinical trials as an adjunct to immune checkpoint blockade (ICB) to improve therapy outcome by concomitantly targeting these two immunosuppressive mechanisms (Lemos et al., 2019; Platten et al., 2019). However, the first phase III clinical trial failed (Long et al., 2019), challenging the inventors' knowledge of Trp catabolism in cancer (Muller et al., 2019; Platten et al., 2019). A molecular understanding of this disappointing result should reveal new opportunities for immunotherapy.

The inventors thus hypothesized that other pathways that activate the AHR could constitute a resistance mechanism against IDO1 inhibitors. To identify new mediators of AHR activation in human tumors, a straightforward approach would be to assess the association of metabolic enzymes beyond IDO1 and TDO2 with the expression of AHR target genes. However, the context-specificity of AHR target gene expression (Rothhammer and Quintana, 2019), which varies greatly across different tissues and ligands, impedes pan-cancer analysis of AHR activity. To meet this challenge, the inventors developed an AHR signature that enables detection of AHR activity, irrespective of cell type or ligand. This pan-tissue AHR signature allows to assess whether other Trp-degrading enzymes beyond IDO1 and TDO2 activate the AHR in human cancers.

The inventors identified interleukin-4 induced 1 (IL4I1) as a major AHR-activating enzyme in human cancer, which associates more frequently with AHR activity than IDO1 or TDO2. IL4I1 enhances tumor cell migration and suppresses anti-tumor immunity. As the key enzyme of a novel Trp catabolic pathway in humans, IL4I1 activity yields indole metabolites and KynA that act as AHR agonists. ICB induces IL4I1, likely mediating resistance to IDO1 inhibitors in combinatorial therapies. Thus, IL4I1 targeting constitutes a new strategy to relieve resistance to immunotherapy and opens new avenues for AHR research. This notion could be generalized to therapies that would stimulate the immune system, which may comprise conventional therapies leading to immune activation, for example chemotherapy and radiotherapy, in addition to targeted therapies, for example kinase inhibitors.

In a first aspect thereof, the present invention relates to method for detecting and/or diagnosing cancer in a patient comprising detecting the change in IL4I1 expression and/or enzymatic activity of IL4I1 in a sample obtained from said patient, and diagnosing and/or detecting cancer in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a patient or patient group, and wherein the method further comprises detecting the activity and/or expression of AHR in a sample obtained from said patient, wherein said activity and/or expression of AHR is modulated, preferably increased, when compared to a control.

In a preferred embodiment, the detecting enzymatic activity of IL4I1 in said sample comprises the detection of the amount and/or concentration of IL4I1 metabolites in the sample. More preferably, detecting the enzymatic activity of IL4I1 comprises the use of chromatography, NMR, metabolite sensors, antibodies, ELISAs, enzymatic assays, colorimetric assays, fluorescence assays or H₂O₂ or ammonia detection, and/or detecting expression using genetic tools, such as chip analysis and primers and probes and PCR analysis, or detecting the amount of protein of IL4I1 using, for examples, antibodies, in samples, like biological fluids and cells/tissue samples.

In a preferred embodiment, the method for detecting the activity and/or expression of AHR comprises determining an aryl hydrocarbon receptor (AHR) activation signature as disclosed in WO2020/201825, or AHR nuclear translocation, or the activity of cytochrome P-450 enzymes or the binding of AHR-ARNT to dioxin-responsive elements (DRE) using reporter assays.

In one aspect, said method comprises detecting the expression and/or enzymatic activity or biological function of IL4I1 in a cell/tissue/biological fluid, wherein a change in the expression, enzymatic activity or biological function in said compartments, in particular expression or activation, when compared to a healthy or other suitable control sample, indicates cancer.

Such a change can be at least about 5%, at least about 10%, at least about 20%, at least about 30%, at least about 40%, at least about 50%, at least about 60%, at least about 70%, at least about 80% or at least about 90% or more of up or down regulation of expression or enzymatic activity or biological function, when compared with a suitable control, such as the value in a healthy cell or a sample derived from a healthy person or group of individuals, or when compared to an internal standard, like a housekeeping gene. In the context of the present invention, the term “about” shall mean +/−5% of the given value, unless indicated otherwise.

In a second aspect thereof, the present invention provides for detecting increased tumor cell motility in a cancer patient comprising detecting the activity and/or expression of IL4I1 in a sample obtained from said patient, and detecting increased tumor cell motility in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group and wherein said method further comprises detecting the activity and/or expression of in a sample obtained from said patient, wherein said activity and/or expression of AHR is modulated, preferably increased, when compared to a control.

The inventors show here that IL4I1 is expressed not only in immune cells (Carbonnelle-Puscian et al., 2009), but also in cancer cells that do not derive from the immune system. For the first time, the inventors link IL4I1 to tumor intrinsic malignant properties, including cancer cell migration and metastasis.

In a third aspect thereof, the present invention provides method for predicting, monitoring or detecting the effect of cancer immune therapy in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for and/or indicates a reduced effect and/or immune evasion of said cancer immune therapy in said patient, immune therapy; and wherein the method further comprises detecting the activity and/or expression of AHR in a sample obtained from said patient, wherein said activity and/or expression of AHR is modulated, preferably increased, when compared to a control; and wherein preferably said increase of said activity and/or expression of said IL4I1 and/or said activity and/or said modulation of the activity and/or expression of AHR, is detected in response to an immune therapy in said patient.

In a preferred embodiment, the cancer immune therapy comprises a combination of immune therapy with IL4I1 modulators, AHR modulators, and/or any conventional cancer treatments.

In a fourth aspect thereof, the present invention provides a method for detecting resistance against a cancer treatment comprising immune therapy in a patient comprising detecting the activity and/or expression of IL4I1 in a sample obtained from said patient, and detecting resistance against a cancer treatment comprising immune therapy in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, and wherein the method further comprises detecting the activity and/or expression of AHR in a sample obtained from said patient, wherein said activity and/or expression of AHR is modulated, preferably increased, when compared to a control.

In a preferred embodiment, method for detecting resistance against a cancer treatment comprising immune therapy wherein the immune therapy comprises immune checkpoint blockade (ICB) and/or IDO1 inhibitors.

In a fifth aspect thereof, the present invention provides a method for predicting survival in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for a reduced survival in said patient, and wherein the method further comprises detecting the activity and/or expression of AHR in a sample obtained from said patient, wherein said activity and/or expression of AHR is modulated, preferably increased, when compared to a control.

In all aspects of the invention, the detection of the I4I1 activity comprises detecting IL4I1 metabolites, in particular IL4I1 tryptophan metabolites such as, for example I3P-derived metabolites, such as KynA, indole-3-acetic acid (IAA), indole-3-aldehyde (I3A) and/or indole-3-lactic acid (ILA) and indole-3-carbinol.

In a most preferred embodiment, the d the activity and/or expression of AHR comprises determining an aryl hydrocarbon receptor (AHR) activation signature as disclosed in WO2020/201825, or AHR nuclear translocation, or the activity of cytochrome P-450 enzymes or the binding of AHR-ARNT to dioxin-responsive elements (DRE) using reporter assays.

Another important aspect of the present invention relates to a diagnostic kit comprising materials for performing a method according to the present invention in one or separate containers, optionally together with auxiliary agents and/or instructions for performing said method. Another important aspect of the present invention then relates to the use of said diagnostic kit in a method according to the present invention.

Generally preferred are the human variants of the biomarker IL4I1 and/or AHR, or closely related species, like the ones from other primates or mammals, such as rodents.

Other aspects and advantages can be readily derived from reading the following description and the non-limiting examples.

The present inventors—while investigating the role of tryptophan degrading enzymes in modulating AHR activity—discovered that the expression of IL4I1, a tryptophan-degrading enzyme expressed in human cancers and not yet implicated in AHR activation, correlated significantly with AHR target gene expression. Gene expression analyses and AHR nuclear translocation established IL4I1 to activate the AHR via the production of tryptophan metabolites including indole-3-pyruvic acid (I3P) and kynurenic acid (see Table 1).

In the context of the present invention, it was found that the modulation of downstream effects of IL4I1, mainly comprising AHR modulation, is caused by the metabolites that are the result of the enzymatic activity or biological function of IL4I1 in a cell/tissue. These metabolites are then, for example, also found in biological fluids, which allows for a more convenient detection of these oncometabolites. I3P and, in particular metabolites derived from I3P represent novel oncometabolites that mediate IL4I1 and AHR-driven malignant properties. Even though it is known that IL4I1 degrades tryptophan in human cells, the inventors have shown for the first time (FIGS. 5 and 6) that the degradation of tryptophan by IL4I1 leads to the accumulation of oncometabolites (I3P and its derivatives) capable of activating the AHR. Therefore, the inventors have demonstrated that IL4I1 is a hitherto unknown source of AHR agonists. Also the inventors have shown in FIGS. 5A and 6C that IL4I1 increases KynA. This finding directly links IL4I1 with the generation of the AHR agonist KynA, further underpinning the importance of IL4I1 for AHR signaling.

Preferably, the detection of said enzymatic activity or biological function of IL4I1 in said sample comprises the detection of the amount and/or concentration of IL4I1 metabolites in said sample. These metabolites are selected from metabolites derived from the conversion of IL4I1 of phenylalanine, tyrosine and/or tryptophan, such as, for example, phenylpyruvic acid (PP), hydroxyphenylpyruvic acid (HPP), indole-3-pyruvic acid (I3P), 2-phenylacetic acid, phenyllactic acid, 4-hydroxybenzaldehyde, 2-hydroxy-2-phenylacetic acid, 4-hydroxyphenyllactic acid, and in particular the I3P derivatives indole-3-acetic acid (IAA), indole-3-aldehyde (I3A) and indole-3-lactic acid (ILA), 4-hydroxyquinoline-2-carboxylic acid (KynA), 1,3-di(1H-indole-3-yl)acetone, (3Z)-1-(1H-indole-3-yl)-3-indole-3-ylidenepropan-2-one, indole-3-carboxylic acid, oxidized indole-3-acetic acid, and indole-3-carbinol and/or the amino acids or amino acid metabolites L-valine, L-isoleucine, L-leucine, L-alanine, L-glutamic acid, L-methionine, L-glutamine, 4-methylsulfanyl-2-oxobutanoate, alpha-keto-isoleucine, alpha-ketoisovalerate, alpha-ketoisocaproic acid, L-proline, and alpha-ketoglutaric acid, and any of the above in combination with ammonia and/or H₂O₂.

In the methods according to the present invention detecting the enzymatic activity or biological function of IL4I1, in particular the generation of metabolites as disclosed herein, can be performed in any suitable manner, including is the use of a peroxide cleavable substrate that can be detected.

In another aspect thereof, the present invention provides for detecting increased tumor cell motility in a cancer patient comprising detecting the activity and/or expression of IL4I1 in a sample obtained from said patient, and detecting increased tumor cell motility in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group and wherein said method further comprises detecting the activity and/or expression of in a sample obtained from said patient, wherein said activity and/or expression of AHR is modulated, preferably increased, when compared to a control.

The inventors show here that IL4I1 is expressed not only in immune cells (Carbonnelle-Puscian et al., 2009), but also in cancer cells that do not derive from the immune system. For the first time, the inventors link IL4I1 to tumor intrinsic malignant properties, including cancer cell migration and metastasis.

Preferably, said cancer is characterized by a modulation, such as an increase of the activity and/or expression of AHR, and/or is selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia. In some embodiments, the cancer is selected from Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM).

IL4I1 promotes tumor cell motility. To test IL4I1's contribution to this phenotype and the functional outcomes of the AHR, the inventors investigated the role of IL4I1 in GBM cell motility. Ectopic IL4I1 increased the motility of AHR-proficient cells, but not of AHR-deficient cells. In keeping with this result, the IL4I1-mediated motility of AHR-proficient cells was reversed by the AHR inhibitor SR1. The finding that IL4I1 acts through the AHR, with its major outputs in immunity and cancer cell migration, enabled the inventors to identify IL4I1 as a driver of cancer cell motility.

AHR-driven motility contributes to the malignant properties of gliomas and other cancer entities, but has so far mainly been linked to its upstream metabolic regulators IDO1 and TDO2. The inventors show here that IL4I1 is of greater importance in cancer than IDO1 and TDO2, and this strong negative association with glioma survival is at least in part due to IL4I1's pro-migratory effects. IL4I1's migratory outcomes do not only explain its importance in glioma but also underlie its impact in metastatic cancers, such as melanoma. In this context, it is important to note that unlike IDO1 and TDO2, IL4I1 is secreted. Thus, in addition to being detected more conveniently in assays, IL4I1 might enhance a systemic pro-metastatic environment that allows metastatic cells to migrate and protects them from immune destruction.

Preferably, said detecting of said activity and/or expression comprises detecting the activity and/or expression of AHR, wherein an increase in said activity and/or expression of AHR when compared to a control is indicative for a modulation, preferably an increase of the activity of IL4I1. The activity of IL4I1 can then, for example, be determined using the metabolites and test therefore as described above.

Expression can be detected using genetic tools, such as chip analysis and primers and probes and PCR analysis, or detecting the amount of protein of IL4I1 using, for examples, antibodies, in samples, like biological fluids and cells/tissue samples.

While the state of the art has speculated about a possible role of IL4I1 in metastasis, these reports have been made in the context of large chip expression signatures, and also lack the important context of AHR and IL4I1, as well as the metabolic functions of IL4I1.

Another aspect of the invention then relates to a method for predicting, monitoring or detecting the effect of cancer immune therapy in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for and/or indicates a reduced effect and/or immune evasion of said cancer immune therapy in said patient, immune therapy; and wherein the method further comprises detecting the activity and/or expression of AHR in a sample obtained from said patient, wherein said activity and/or expression of AHR is modulated, preferably increased, when compared to a control; and wherein preferably said increase of said activity and/or expression of said IL4I1 and/or said activity and/or said modulation of the activity and/or expression of AHR, is detected in response to an immune therapy in said patient.

Importantly, the inventors show that IL4I1 is expressed not only in immune cells (Carbonnelle-Puscian et al., 2009), but also in cancer cells that do not derive from the immune system. The inventors show that catabolism of Trp by the L-amino-acid oxidase IL4I1 elicits major effects in immunity and cancer by signaling through the AHR. Until now IL4I1 has been merely implicated in immune regulatory functions that have been attributed either to the depletion of amino acids, the formation of H₂O₂ or an enzyme-independent function of IL4I1 (Molinier-Frenkel et al., 2019).

Thus, IL4I1 targeting constitutes a new strategy to relieve resistance to immunotherapy.

Expression can be detected using genetic tools, such as chip analysis and primers and probes and PCR analysis, or detecting the amount of protein of IL4I1 using, for examples, antibodies, in samples, like serum samples. The term “immunotherapy” as used herein shall include any artificial stimulation of the immune system to treat cancer, such as cellular immunotherapy, cancer vaccination(s), anti-cancer antibody treatments, immune cell engagers, and cytokine treatments. Particularly mentioned shall be a cancer treatment comprising immune checkpoint blockade (ICB), such as, for example, comprising the use of IDO1 inhibitors (see, for example, Prendergast G C, et al. Discovery of IDO1 Inhibitors: From Bench to Bedside. Cancer Res. 2017; 77(24):6795-6811, and Cheong J E, et al. A patent review of IDO1 inhibitors for cancer. Expert Opin Ther Pat. 2018 April; 28(4):317-330), and/or antibodies selected from anti-CTLA-4, anti-PD-1, anti-PDL-1, anti LAG3 and anti BTLA, and adoptive immunotherapy.

Particularly preferred is a method according to the present invention, wherein said increase of said activity and/or expression of said IL4I1 is detected in response to an immunotherapy in said patient. In the context of the present invention, it was shown for the first time that resistance to immunotherapy is actively linked to the increased activity and/or expression of IL4I1 and the IL4I1-AHR axis.

Another aspect of the invention then relates to a method for detecting resistance against a cancer treatment comprising immune therapy in a patient comprising detecting the activity and/or expression of IL4I1 in a sample obtained from said patient, and detecting resistance against a cancer treatment comprising immune therapy in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, and wherein the method further comprises detecting the activity and/or expression of AHR in a sample obtained from said patient, wherein said activity and/or expression of AHR is modulated, preferably increased, when compared to a control.

Preferably, said immune therapy treatments comprise at least one single immune therapy intervention, for example ICB treatment that comprises the use of IDO1 inhibitors (see, for example, Prendergast G C, et al. Discovery of IDO1 Inhibitors: From Bench to Bedside. Cancer Res. 2017; 77(24):6795-6811, and Cheong J E, et al. A patent review of IDO1 inhibitors for cancer. Expert Opin Ther Pat. 2018 April; 28(4):317-330) and/or antibodies selected from anti-CTLA-4, anti-PD-1, anti-PDL-1, anti LAG3 and anti BTLA, used as a single line of treatment or in combination with one or more cancer therapeutic, for example kinase inhibitors, nuclear receptor antagonists, other chemotherapeutic agents or radiotherapy.

Similar to the aspect above, IL4I1 targeting constitutes a new strategy to relieve resistance to immunotherapy, in this case in the context of ICB-related cancer treatment. As mentioned above, the activity of IL4I1 can then, for example, be determined using the metabolites and tests as described above. Expression can be detected using genetic tools, such as chip analysis and primers and probes and PCR analysis, or detecting the amount of protein of IL4I1 using, for examples, antibodies, in samples, like biological fluids or tissue samples. Particularly preferred is a method according to the present invention, wherein said increase of said activity and/or expression of said IL4I1 is detected in response to immune therapy such as immune checkpoint blockade (ICB), IDO1 inhibitors or both. In the context of the present invention, it was shown for the first time that resistance to immunotherapy is actively linked to the increased activity and/or expression of IL4I1 and the IL4I1-AHR axis. The consequence of a positive finding based on this method would be the use of an IL4I1 inhibitor, or an AHR modulator for therapy.

Another aspect of the invention then relates to a method of stratifying cancer patients into a high survival group and a low survival group comprising detecting the activity and/or expression of IL4I1 in a sample obtained from said cancer patients, wherein if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the low survival group and wherein if said expression or enzymatic activity of said IL4I1 is unchanged or decreased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the high survival group. In a preferred embodiment, the increase of said activity and/or expression of said IL4I1 is detected in response to an immune therapy in said patient.

In a more preferred embodiment, the patients in the high survival group are immune therapy responders and patients in the low survival group are immune therapy non-responders, in particular immune checkpoint blockade (ICB) non responders and/or IDO1 inhibitors non-responders.

Similarly, another aspect of the invention relates to a method of stratifying cancer patients into responders and non-responders group to immune therapy comprising detecting the activity and/or expression of IL4I1 in a sample obtained from said cancer patients after said patients are subject to immune therapy, wherein if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the non-responders group and wherein if said expression or enzymatic activity of said IL4I1 is unchanged or decreased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the responders group. Ina preferred embodiment, the control sample is from the same patients prior to being subject to said immune therapy.

More preferably, the method further comprises detecting the activity and/or expression of AHR in a sample obtained from said patient, wherein said activity and/or expression of AHR is modulated, preferably increased, when compared to a control. In a most preferred embodiment, the modulation of the activity and/or expression of said AHR is detected in response to immune therapy in said patient. The immune therapy can advantageously comprise immune checkpoint blockade (ICB) and/or IDO1 inhibitors.

Another aspect of the invention relates to a method of treating a cancer in a subject in need thereof wherein the method comprises detecting the activity and/or expression of IL4I1 in a sample obtained from said patient, and wherein at least one IL4I1 inhibitor and/or at least one AHR modulator is administered in an effective amount to said subject if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group. In a preferred embodiment, the method further comprises detecting the activity and/or expression of AHR in a sample obtained from said patient, wherein said activity and/or expression of AHR is modulated, preferably increased, when compared to a control.

In a preferred embodiment, the increase of said activity and/or expression of said IL4I1 and/or said modulation of the activity and/or expression of AHR is detected in response to an immune therapy in said patient. More preferred is a method of treating a cancer comprising an immune therapy, in particular a immune therapy involving the use of at least one immune checkpoint inhibitor.

In a preferred embodiment. the one immune checkpoint inhibitor is an anti-CTLA4, anti-PD-L1, anti-PD1, anti-TB/13, anti-TIGIT, anti-LAG3 or a combination thereof.

In a more preferred embodiment, the immune therapy comprises a combination of immune therapy with inhibitors of IDO1, inhibitors of TDO2, AHR modulators and/or any conventional cancer treatments.

Preferred examples of IL4I1 inhibitors are 3-phenyl-2-piperidin-1-ylpropanoic acid, 2-(4-methylpiperazin-1-yl)-3-phenylpropanoic acid, 2-(diethylamino)-3-phenylpropanoic acid, 3-(2,6-dichlorophenyl)-2-piperidin-1-ylpropanoic acid, 3-phenyl-2-(propylamino)propanoic acid, 2-anilino-3-phenylpropanoic acid, L-Phenylalanine N-2-propen-1-yl-3-(trifluoromethyl), L-Phenylalanine, 4-cyano-N-phenyl, N-Propyl-L-phenylalanine, N-Phenyl-L-phenylalanine, 2-amino-3-phenyl-propionic acid. ethyl ester, 2-acteylamino-3-phenyl-propionic acid or 3-(2-pyridyl)-alanine.

Preferred examples of modulators of AHR are 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2,1,3-dichloro-5-[(1E)-2-(4-methoxyphenyl)ethenyl]-benzene, α-Naphthoflavone, 6,2′,4′-Trimethoxyflavone, CH223191, a tetrahydropyridopyrimidine derivative, StemRegenin-1, CH223191, GNF351, CB7993113 HP163, PX-A590, PX-A548, PX-A275, PX-A758, PX-A446, PX-A24590, PX-A25548, PX-A25275, PX-A25758, PX-A26446, an Indole AHR inhibitor, or an oxazole-containing (OxC) compound.

In a more preferred embodiment, the cancer is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells.34.

In a preferred embodiment, the IL4I1 inhibitor is selected from small molecules (e.g., 220307 and CC-668) as well as potentially IL4I1 blocking antibodies.

In a preferred embodiment, the treatment of the cancer is for a cancer characterized by a modulation, such as an increase of the activity and/or expression of AHR, and/or is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia. In some embodiments, the cancer is selected from Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells, and any relapsing and metastasizing forms thereof.

In a more preferred embodiment, the immunotherapy comprises a combination of immunotherapy such as immune checkpoint inhibitor with inhibitors of IDO1, inhibitors of TDO2, AHR modulators and/or any other cancer treatments, in particular conventional cancer treatments.

Preferred examples of IDO1 inhibitors are -methyl-L-tryptophan, Epacadostat, PX-D26116, navoximod, PF-06840003, NLG-919A, BMS-986205, INCB024360A, KHK2455, LY3381916, MK-7162. Preferred examples of TDO2 inhibitors are (680C91, LM10,4-(4-fluoropyrazol-1-yl)-1,2-oxazol-5-amine, fused imidazo-indoles, indazoles). Also preferred are IDO/TDO inhibitors such as HTI-1090/SHR9146, DN1406131, RG70099, EPL-1410

In a preferred embodiment, the AHR modulators can be selected from small organic chemical compound, generally having a molecular weight of less than 2000 daltons, 1500 daltons, 1000 daltons, 800 daltons, or 600 daltons.

In a preferred embodiment, conventional cancer treatments comprise surgery, radiation therapy, chemotherapy, hormone therapy and/or stem cell transplant.

In a preferred embodiment, the AHR modulators are inhibitor of AHR or AHR Nuclear Translocator (ARNT) activity or expression such as an antagonist of AHR as well as antibodies shRNA or a siRNA, blocking the AI-ER pathway.

In some embodiments, an AHR modulator comprises a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2,1,3-dichloro-5-[(1E)-2-(4-methoxyphenyl)ethenyl]-benzene, α-Naphthoflavone, 6,2′,4′-Trimethoxyflavone, CH223191, a tetrahydropyridopyrimidine derivative, StemRegenin-1, CH223191, GNF351, CB7993113 HP163, PX-A590, PX-A548, PX-A275, PX-A758, PX-A446, PX-A24590, PX-A25548, PX-A25275, PX-A25758, PX-A26446, an Indole AHR inhibitor, and an oxazole-containing (OxC) compound.

In some embodiments, a direct AHR modulator comprises Omeprazole, Sulindac, Leflunomide, Tranilast, Laquinimod, Flutamide, Nimodipine, Mexiletine, 4-Hydroxy-Tamoxifen, Vemurafenib etc.

In some embodiments, the modulation of the levels of AHR agonists or antagonists is mediated through one or more of the following:

(a) regulation of enzymes modifying AHR ligands e.g. the cytochrome p450 enzymes by e.g. cytochrome p450 enzyme inhibitors including 3′methoxy-4′nitroflavone (MNF), alpha-naphthoflavone (a-NF), fluoranthene (FL), phenanthrene (Phe), pyrene (PY) etc.

(b) regulation of enzymes producing AHR ligands including direct and indirect inhibitors/activators/inducers of tryptophan-catabolizing enzymes e.g. IDO1 pathway modulators (indoximod, NLG802), IDO1 inhibitors (1-methyl-L-tryptophan, Epacadostat, PX-D26116, navoximod, PF-06840003, NLG-919A, BMS-986205, INCB024360A, KHK2455, LY3381916, MK-7162, TDO2 inhibitors (680C91, LM10,4-(4-fluoropyrazol-1-yl)-1,2-oxazol-5-amine, fused imidazo-indoles, indazoles), dual IDO/TDO inhibitors (HTI-1090/SHR9146, DN1406131, RG70099, EPL-1410), immunotherapy including immune checkpoint inhibition, vaccination, and cellular therapies, chemotherapy, immune stimulants, radiotherapy, exposure to UV light, and targeted therapies such as e.g. imatinib etc.

In some embodiments, indirect AHR modulators affect AHR activation through modulation of the expression of the AHR including e.g. HSP 90 inhibitors such as 17-allylamino-demethoxygeldanamycin (17-AAG), celastrol.

In some embodiments, indirect AHR modulators affect AHR activation by affecting binding partners/co-factors modulating the effects of AHR including e.g. estrogen receptor alpha (ESR1).

Examples of AHR modulators are listed in U.S. Pat. No. 9,175,266, US2019/225683, WO2019101647A1, WO2019101642A1, WO2019101643A1, WO2019101641A1, WO2018146010A1, AU2019280023A1, WO2020039093A1, WO2020021024A1, WO2019206800A1, WO2019185870A1, WO2019115586A1, EP3535259A1, WO2020043880A1 and EP3464248A1, all of which are incorporated by reference in their entirety.

In a preferred embodiment, the treatment of the cancer is for a cancer characterized by a modulation, such as an increase of the activity and/or expression of AHR, and/or is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia. In some embodiments, the cancer is selected from Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells, and any relapsing and metastasizing forms thereof.

Another aspect of the invention then relates to a method for predicting survival in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for a reduced survival in said patient, and wherein the method further comprises detecting the activity and/or expression of AHR in a sample obtained from said patient, wherein said activity and/or expression of AHR is modulated, preferably increased, when compared to a control.

As mentioned above, the activity of IL4I1 can then, for example, be determined using the metabolites and tests as described above. Expression can be detected using genetic tools, such as chip analysis and primers and probes and PCR analysis, or detecting the amount of protein of IL4I1 using, for examples, antibodies, in samples, like biological fluids or tissue samples.

The inventors show here that IL4I1 and the IL4I1-AHR axis is of greater importance in cancer such as for glioma survival compared to IDO1 and TDO2, and this strong negative association with glioma survival is at least in part due to IL4I1's pro-migratory effects. As an example, the inventors analyzed the association of the three AHR-activating enzymes IL4I1, IDO1, and TDO2, with overall survival probability in GBM. High IL4I1 levels associated with reduced overall survival (FIG. 3D). In fact, GBM patients with high tumoral IL4I1 expression (low survival group) had a 2.3 times higher risk of death compared to patients with low IL4I1 expression (high survival group) (FIG. 3D, and Table 2). High TDO2 levels associated with a 1.5 times higher risk of death, while no significant survival difference was associated with IDO1 transcript levels (FIG. 3D and Table 2). Similarly, in low grade glioma (LGG) high expression of IL4I1 (low survival group), IDO1 and TDO2 were all associated with a higher risk of death (FIG. 3E). The risk increase was highest for IL4I1 expression (3.3 fold), followed by IDO1 (2.4 fold), and TDO2 (1.6 fold) (FIG. 3E, and Table 2). Similarly, patients with high expression levels of IL4I1, in additional TCGA tumors show significant worse overall survival outcome compared to low IL4I1 expression (FIG. 9).

As also mentioned above, preferred is a method according to the present invention, wherein said detecting of said activity comprises detecting IL4I1 metabolites (oncometabolites), in particular IL4I1 tryptophan metabolites such as, for example I3P and/or the I3P derivatives indole-3-acetic acid (IAA), indole-3-aldehyde (I3A) and indole-3-lactic acid (ILA). I3P, and in particular the downstream metabolites thereof as disclosed herein, represent novel oncometabolites that mediate IL4I1 and AHR-driven malignant properties.

Preferably, the detection of said enzymatic activity or biological function of IL4I1 in said sample comprises the detection of the amount and/or concentration of IL4I1 metabolites in said sample. These metabolites are selected from metabolites derived from the conversion of IL4I1 of phenylalanine, tyrosine and/or tryptophan, such as, for example, phenylpyruvic acid (PP), hydroxyphenylpyruvic acid (HPP), indole-3-pyruvic acid (I3P), 2-phenylacetic acid, phenyllactic acid, 4-hydroxybenzaldehyde, 2-hydroxy-2-phenylacetic acid, 4-hydroxyphenyllactic acid, and in particular the I3P derivatives indole-3-acetic acid (IAA), indole-3-aldehyde (I3A) and indole-3-lactic acid (ILA), 4-hydroxyquinoline-2-carboxylic acid (KynA), 1,3-di(1H-indole-3-yl)acetone, (3Z)-1-(1H-indole-3-yl)-3-indole-3-ylidenepropan-2-one, indole-3-carb oxylic acid, oxidized indole-3-acetic acid, and indole-3-carbinol and/or the amino acids or amino acid metabolites L-valine, L-isoleucine, L-leucine, L-alanine, L-glutamic acid, L-methionine, L-glutamine, 4-methylsulfanyl-2-oxobutanoate, alpha-keto-isoleucine, alpha-ketoisovalerate, alpha-ketoisocaproic acid, L-proline, and alpha-ketoglutaric acid, and any of the above in combination with ammonia and/or H₂O₂.

In the methods according to the present invention detecting the enzymatic activity or biological function of IL4I1 can be performed in any suitable manner, preferred is the use of chromatography, NMR, metabolite sensors, antibodies, ELISAs, enzymatic assays, colorimetric assays, fluorescence assays and/or H₂O₂ detection or ammonia detection.

Another aspect of the invention then relates to a method according to the present invention, wherein said detecting of said activity comprises detecting the activity and/or expression of AHR, wherein an increase in said activity and/or expression of AHR when compared to a suitable control is indicative for an increase of the activity of IL4I1. Detecting the activity and/or expression of AHR can be achieved in accordance with methods as disclosed in the state of the art in particular in the PCT application WO2020/201825 (in the name of DEUTSCHES KREBSFORSCHUNGSZENTRUM STIFTUNG DES ÖFFENTLICHEN RECHTS), and the examples herein, and also can involve detection using genetic tools, such as chip analysis and primers and probes and PCR analysis, or detecting the amount of protein of IL4I1 using, for examples, antibodies, in samples, like biological fluids and cell/tissue samples. The whole content of the PCT application WO2020/201825 as published in incorporated by reference into the present application.

In the context of the present invention, any biological sample comprising the marker protein IL4I1 (or functionally relevant parts thereof), or a sample comprising cells, comprising the marker protein IL4I1 (or functionally relevant parts thereof), e.g. obtained from a cancer patient, or a sample comprising at least one of the metabolites produced downstream of IL4I1, for example as shown in Table 1, can be used, as long as it contains (or is presumed to contain) at least one of the biomarker(s) (including AHR or AHR targets) or is presumed to have a perturbed AHR activity profile, to be used in the analysis.

Preferably, the biological sample is selected from a sample comprising biological fluids comprising biomarkers (in particular the present oncometabolites, AHR, and/or IL4I1), cells, a suitable sample comprising biological fluids, human cells, tissues, whole blood, cell lines, cellular supernatants, primary cells, IPSCs, hybridomas, recombinant cells, stem cells, and cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cells or tissues. The sample can also be selected from tumor tissue (tumor or metastases), biopsies, whole blood, peripheral blood, or fractions thereof, serum, buffy coat, lymphatic fluid, urine, bone marrow, heparinized whole blood, and frozen samples thereof, such as frozen heparinized whole blood. The cells to be used in the methods according to the present invention can be recombinant or non-recombinant, and express cell-foreign proteins, depending on the desired purpose and circumstances. Totipotent human embryonic stem cells may be excluded, if necessary. The sample can also be a combined sample from a group of subjects, for example, a group of healthy subjects, or a patient group.

In the methods according to the present invention the control sample can be selected from, for example, a sample from a healthy subject, an earlier (prior) sample from the same or a different similar patient, and/or from a group of subjects/patients. Preferred is a method according to the present invention, wherein said subject is selected from a mammalian subject, in particular a human subject, in particular a human patient suffering from an IL4I1- and/or AHR-related physiological or pathological condition. Said control sample can be selected from a sample as described above.

Preferred is a method according to the present invention, wherein one or more reference genes could be used in determining the increase of the activity and/or expression of IL4I1.

Preferred is a method according to the present invention, wherein said cancer is characterized by a modulation, such as an increase or a decrease of the activity and/or expression of IL4I1 and/or AHR, and/or is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia. In some embodiments, the cancer is selected from Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells, and any relapsing and metastasizing forms thereof.

Preferred is a method according to the present invention, further comprising the step of a stratification of said patient to a disease and/or treatment group, for example a treatment group receiving suitable IL4I1 inhibitors or AHR modulators. Said stratification allows defining subgroups of patients with distinct mechanisms of disease, or particular responses to treatments in order to identify and develop treatments that are effective for particular groups of patients.

In another aspect of the present invention, the invention then relates to a diagnostic kit comprising materials for performing a method according to the present invention as herein in one or separate containers, optionally together with auxiliary agents and/or instructions for performing said methods according to the present invention.

Another aspect of the present invention then relates to the use of a diagnostic kit according to the present invention in a method according to the present invention.

In another preferred aspect of the present invention, the invention then relates to a method of treating and/or preventing an IL4I1-related disease or condition, in particular cancer, such as AHR-related cancer, in a cell in a patient in need of said treatment, comprising performing a method according to the present invention, and providing a suitable treatment to said patient, wherein said treatment is based, at least in part, on the results of the method according to the present invention, such as a stratification.

Particularly preferred is a method according to the present invention, wherein said treatment comprises an inhibition of the expression and/or enzymatic activity of IL4I1, more preferably leading to a reduction of IL4I1 expression or IL4I1 associated oncometabolites, possibly in combination with immunotherapy, and/or the co-inhibition of IDO1 or TDO2, to achieve efficient de-repression of anti-tumor immunity. Consequently, in some embodiments said treatment comprises AHR modulation, possibly in combination with immunotherapy, and/or the co-inhibition of IDO1 or TDO2, to achieve efficient de-repression of anti-tumor immunity.

In addition to the above, in the methods of the present invention, in general the IL4I1 and/or AHR can be detected and/or determined using any suitable assay. Detection is usually directed at the qualitative information (“marker yes-no”), whereas determining involves analysis of the quantity of a marker (e.g. expression level and/or activity). Detection is also directed at identifying for example mutations that cause altered functions of individual markers. The choice of the assay(s) depends on the parameter of the marker to be determined and/or the detection process. Thus, the determining and/or detecting can preferably comprise a method selected from subtractive hybridization, microarray analysis, DNA sequencing, RNA sequencing, qPCR, ELISA, IP, PLA, BiFC, HPLC, WB, enzymatic activity tests, fluorescence detection, cell viability assays, for example an MTT assay, phosphoreceptor tyrosine kinase assays, phospho arrays and proliferation assays, for example the BrdU assay, proteomics, cytokine arrays, and mass spectrometry.

Preferably, the inventive methods are also amenable to automation, and e.g. said activity and/or expression is preferably assessed in an automated and/or high-throughput format. Usually, this involves the use of chips and respective machinery, such as robots.

The inventors report that IL4I1 elicits major effects in immunity and cancer by signaling through the AHR. Until now IL4I1 has been merely implicated in immune regulatory functions that have been attributed either to the depletion of amino acids, the formation of H₂O₂ or an enzyme-independent function of IL4I1 (Molinier-Frenkel et al., 2019).

The inventors show here that IL4I1 is expressed not only in immune cells (Carbonnelle-Puscian et al., 2009), but also in cancer cells that do not derive from the immune system. For the first time, the inventors link IL4I1 to tumor intrinsic malignant properties, including cancer cell migration and metastasis.

The finding that IL4I1 acts through the AHR, with its major outputs in immunity and cancer cell migration, enabled the inventors to identify IL4I1 as a driver of cancer cell motility. AHR-driven motility contributes to the malignant properties of gliomas and other cancer entities, but has so far mainly been linked to its upstream metabolic regulators IDO1 and TDO2 (Chen et al., 2014; D'Amato et al., 2015; Gabriely et al., 2017; Novikov et al., 2016; Opitz et al., 2011; Xiang et al., 2019). The inventors show here that IL4I1 is of greater importance in glioma survival than IDO1 and TDO2, and this strong negative association with glioma survival is at least in part be due to IL4I1's pro-migratory effects.

IL4I1's migratory outcomes do not only explain its importance in glioma but may also underlie its impact in metastatic cancers such as melanoma. In this context, it is important to note that unlike IDO1 and TDO2, IL4I1 is secreted (Boulland et al., 2007). Thus, IL4I1 might enhance a systemic pro-metastatic environment that allows metastatic cells to migrate and protects them from immune destruction. In support of this idea, cancer patients exhibit increased systemic concentrations of IL4I1-derived metabolites (Fong et al., 2011; Huang et al., 2016; Locasale et al., 2012).

Next to assigning new functions to IL4I1, its stimulatory effect on AHR also adds key aspects to IL4I1's known involvement in immunity (Molinier-Frenkel et al., 2019). Specifically, the enrichment of MDSCs and Tregs in IL4I1-expressing tumors is likely attributable to the AHR, as it promotes Treg differentiation (Esser et al., 2009; Gagliani et al., 2015; Marshall and Kerkvliet, 2010; Mezrich et al., 2010; Quintana et al., 2008) and MDSC recruitment to tumors (Neamah et al., 2019).

The co-existence of IL4I1, IDO1, and TDO2 as activators of the AHR underscores the biological importance of this metabolic checkpoint. In the context of tumor therapy, the stronger effects of IL4I1 on the AHR—as compared to IDO1 or TDO2—highlight IL4I1 as a key drug target upstream of the AHR. It remains to be investigated whether the co-inhibition of IDO1 or TDO2 (see, for example, WO 2017/107979A1) is necessary in order to avoid therapy escape in response to IL4I1 blockade (see, for example, Prendergast G C, et al. Discovery of IDO1 Inhibitors: From Bench to Bedside. Cancer Res. 2017; 77(24):6795-6811, and Cheong J E, et al. A patent review of IDO1 inhibitors for cancer. Expert Opin Ther Pat. 2018 April; 28(4):317-330). This is also critical in the context of immunotherapy, which enhances IL4I1 levels along with IDO1 and may therefore require concomitant targeting of both of these enzymes to achieve efficient de-repression of anti-tumor immunity. The inventors therefore advocate the development of IL4I1-targeting drugs as a new avenue for cancer therapy and beyond.

### Definitions

As used herein, the term “about” refers to a variation within approximately ±10% from a given value.

An “AHR signaling modulator” or an “AHR modulator” as used herein, refers to a modulator which affects AHR signaling in a cell. In some embodiments, an AHR signaling modulator exhibits direct effects on AHR signaling. In some embodiments, the direct effect on AHR is mediated through direct binding to AHR. In some embodiments, a direct modulator exhibits full or partial agonistic and/or antagonistic effects on AHR. In some embodiments, an AHR modulator is an indirect modulator.

In some embodiments, an AHR signaling modulator is a small molecule compound. The term “small molecule compound” herein refers to small organic chemical compound, generally having a molecular weight of less than 2000 daltons, 1500 daltons, 1000 daltons, 800 daltons, or 600 daltons.

In some embodiments, an AHR modulator comprises a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2,1,3-dichloro-5-[(1E)-2-(4-methoxyphenyl)ethenyl]-benzene, α-Naphthoflavone, 6,2′,4′-Trimethoxyflavone, CH223191, a tetrahydropyridopyrimidine derivative, StemRegenin-1, CH223191, GNF351, CB7993113 HP163, PX-A590, PX-A548, PX-A275, PX-A758, PX-A446, PX-A24590, PX-A25548, PX-A25275, PX-A25758, PX-A26446, an Indole AHR inhibitor, and an oxazole-containing (OxC) compound.

In some embodiments, a direct AHR modulator comprises:

(a) Drugs: e.g. Omeprazole, Sulindac, Leflunomide, Tranilast, Laquinimod, Flutamide, Nimodipine, Mexiletine, 4-Hydroxy-Tamoxifen, Vemurafenib etc.

(b) Synthethic compounds: e.g. 10-Chloro-7H-benzimidazo[2,1-a]benz[de]isoquinolin-7-one (10-Cl-BBQ), Pifithrin-a hydrobromide,

(c) Natural compounds: e.g., kynurenine, kynurenic acid, cinnabarinic acid, ITE, FICZ, indoles including indole-3-carbinol, indole-3-pyruvate, indole-aldehyde, microbial metabolites, dietary components, quercetin, resveratrol, curcurmin, or

(d) Toxic compounds: e.g. TCDD, cigarette smoke, 3-methylcholantrene, benzo(a)pyrene, 2,3,7,8-tetrachlorodibenzofuran, fuel emissions, halogenated and nonhalogenated aromatic hydrocarbon, pesticides.

In some embodiments, indirect AHR modulators affect AHR activation through modulation of the levels of AHR agonists or antagonists.

In some embodiments, the modulation of the levels of AHR agonists or antagonists is mediated through one or more of the following:

(a) regulation of enzymes modifying AHR ligands e.g. the cytochrome p450 enzymes by e.g. cytochrome p450 enzyme inhibitors including 3¢methoxy-4¢nitroflavone (MNF), alpha-naphthoflavone (a-NF), fluoranthene (FL), phenanthrene (Phe), pyrene (PY) etc. (b) regulation of enzymes producing AHR ligands including direct and indirect inhibitors/activators/inducers of tryptophan-catabolizing enzymes e.g. IDO1 pathway modulators (indoximod, NLG802), IDO1 inhibitors (1-methyl-L-tryptophan, Epacadostat, PX-D26116, navoximod, PF-06840003, NLG-919A, BMS-986205, INCB024360A, KHK2455, LY3381916, MK-7162, TDO2 inhibitors (680C91, LM10,4-(4-fluoropyrazol-1-yl)-1,2-oxazol-5-amine, fused imidazo-indoles, indazoles), dual IDO/TDO inhibitors (HTI-1090/SHR9146, DN1406131, RG70099, EPL-1410), immunotherapy including immune checkpoint inhibition, vaccination, and cellular therapies, chemotherapy, immune stimulants, radiotherapy, exposure to UV light, and targeted therapies such as e.g. imatinib etc.

In some embodiments, indirect AHR modulators affect AHR activation through modulation of the expression of the AHR including e.g. HSP 90 inhibitors such as 17-allylamino-demethoxygeldanamycin (17-AAG), celastrol.

In some embodiments, indirect AHR modulators affect AHR activation by affecting binding partners/co-factors modulating the effects of AHR including e.g. estrogen receptor alpha (ESR1).

Examples of AHR modulators are listed in U.S. Pat. No. 9,175,266, US2019/225683, WO2019101647A1, WO2019101642A1, WO2019101643A1, WO2019101641A1, O2018146010A1, AU2019280023A1, WO2020039093A1, WO2020021024A1, WO2019206800A1, WO2019185870A1, WO2019115586A1, EP3535259A1, WO2020043880A1 and EP3464248A1, all of which are incorporated by reference in their entirety.

As used herein, the phrase “biological sample” refers to any sample taken from a living organism. In some embodiments, the living organism is a human. In some embodiments, the living organism is a non-human animal.

In some embodiments, a biological sample includes, but is not limited to, biological fluids comprising biomarkers, cells, tissues, and cell lines. In some embodiments, a biological sample includes, but is not limited to, primary cells, induced pluripotent cells (IPCs), hybridomas, recombinant cells, whole blood, stem cells, cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cell types thereof.

“Treatment” or “treating” shall mean a reduction and/or amelioration of the symptoms of the disease. An effective treatment achieves, for example, a shrinking of the mass of a tumor and the number of cancer cells. A treatment can also avoid (prevent) and reduce the spread of the cancer, such as, for example, affect metastases and/or the formation thereof. A treatment may be a naïve treatment (before any other treatment of a disease had started), or a treatment after the first round of treatment (e.g. after surgery or after a relapse). In some embodiments, a treatment is a combined treatment, involving, conventional cancer treatments for example, surgery, radiation therapy, chemotherapy, hormone therapy and/or stem cell transplant. In some embodiments, treatment can also modulate auto-immune response, infection and inflammation.

Biological sample refers to a sample suitable for further analysis in particular detection of IL4I1 or AHR activity. Such sample includes but is not limited to biological fluids, mammalian, for example human, cells, tissues, whole blood, cell lines, cellular supernatants, primary cells, IPSCs, hybridomas, recombinant cells, stem cells, and cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cells or tissues, and in particular cancer cells that do not derive from the immune system. Tumor and cancer cells are of particular interest in the present invention.

Control or control sample refers to for example to a biological sample from a healthy subject or group of healthy subjects, a prior sample from the same patient, or a different patient or a patient group. In a preferred embodiment of the invention the control is from the same patient or group of patients than the sample where IL4I1 and/or AHR activity is detected but taken from the patient or group of patients prior to immune therapy.

The present invention thus relates to the following items.

Item 1. A method for detecting and/or diagnosing cancer in a patient comprising detecting the change in IL4I1 expression and/or enzymatic activity of IL4I1 in a sample obtained from said patient, and diagnosing and/or detecting cancer in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a patient or patient group, and wherein the method further comprises detecting the activity and/or expression of AHR in a sample obtained from said patient, wherein said activity and/or expression of AHR is modulated, preferably increased, when compared to a control.

Item 2. The method according to item 1, wherein said detecting enzymatic activity of IL4I1 in said sample comprises the detection of the amount and/or concentration of IL4I1 metabolites in said sample.

Item 3. The method according to item 2, wherein said metabolites are selected from metabolites derived from the conversion of IL4I1 of phenylalanine, tyrosine and/or tryptophan, such as, for example, phenylpyruvic acid (PP), hydroxyphenylpyruvic acid (HPP), indole-3-pyruvic acid (I3P), 2-phenylacetic acid, phenyllactic acid, 4-hydroxybenzaldehyde, 2-hydroxy-2-phenylacetic acid, 4-hydroxyphenyllactic acid, and in particular the I3P derivatives indole-3-acetic acid (IAA), indole-3-aldehyde (I3A) and indole-3-lactic acid (ILA), 4-hydroxyquinoline-2-carboxylic acid (KynA), 1,3-di(1H-indole-3-yl)acetone, (3Z)-1-(1H-indole-3-yl)-3-indole-3-ylidenepropan-2-one, indole-3-carboxylic acid, oxidized indole-3-acetic acid, and indole-3-carbinol and/or the amino acids or amino acid metabolites L-valine, L-isoleucine, L-leucine, L-alanine, L-glutamic acid, L-methionine, L-glutamine, 4-methylsulfanyl-2-oxobutanoate, alpha-keto-isoleucine, alpha-ketoisovalerate, alpha-ketoisocaproic acid, L-proline, and alpha-ketoglutaric acid, and any of the above in combination with ammonia and/or H₂O₂.

Item 4. The method according to any one of items 1 to 3, wherein detecting the enzymatic activity of IL4I1 comprises the use of chromatography, NMR, metabolite sensors, antibodies, ELISAs, enzymatic assays, colorimetric assays, fluorescence assays or H₂O₂ or ammonia detection, and/or detecting expression using genetic tools, such as chip analysis and primers and probes and PCR analysis, or detecting the amount of protein of IL4I1 using, for examples, antibodies, in samples, like biological fluids and cells/tissue samples.

Item 5. A method for detecting increased tumor cell motility in a cancer patient comprising detecting the activity and/or expression of IL4I1 in a sample obtained from said patient, and detecting increased tumor cell motility in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group and wherein said method further comprises detecting the activity and/or expression of in a sample obtained from said patient, wherein said activity and/or expression of AHR is modulated, preferably increased, when compared to a control.

Item 6. A method for predicting, monitoring or detecting the effect of cancer immune therapy in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for and/or indicates a reduced effect and/or immune evasion of said cancer immune therapy in said patient, immune therapy and wherein the method further comprises detecting the activity and/or expression of AHR in a sample obtained from said patient, wherein said activity and/or expression of AHR is modulated, preferably increased, when compared to a control, and wherein preferably said increase of said activity and/or expression of said IL4I1 and/or said activity and/or said modulation of the activity and/or expression of AHR, is detected in response to an immune therapy in said patient.

Item 7. A method for predicting, monitoring or detecting the effect of cancer immune therapy in a cancer patient according to item 6 wherein the cancer immune therapy comprises a combination of immune therapy with IL4I1 modulators, AHR modulators, and/or any other conventional cancer treatments.

Item 8. A method for detecting resistance against a cancer treatment comprising immune therapy in a patient comprising detecting the activity and/or expression of IL4I1 in a sample obtained from said patient, and detecting resistance against a cancer treatment comprising immune therapy in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, and wherein the method further comprises detecting the activity and/or expression of AHR in a sample obtained from said patient, wherein said activity and/or expression of AHR is modulated, preferably increased, when compared to a control.

Item 9. A method for detecting resistance against a cancer treatment comprising immune therapy according to item 8 wherein the immune therapy comprises immune checkpoint blockade (ICB) and/or IDO1 inhibitors.

Item 10. A method for predicting survival in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for a reduced survival in said patient, and wherein the method further comprises detecting the activity and/or expression of AHR in a sample obtained from said patient, wherein said activity and/or expression of AHR is modulated, preferably increased, when compared to a control.

Item 11. The method according to any one of items 1 to 10, wherein said detecting of said activity comprises detecting IL4I1 metabolites, in particular IL4I1 tryptophan metabolites such as, for example I3P-derived metabolites, such as KynA, indole-3-acetic acid (IAA), indole-3-aldehyde (I3A) and/or indole-3-lactic acid (ILA) and indole-3-carbinol.

Item 12. The method according to any one of items 1 to 11, wherein the method for detecting the activity and/or expression of AHR comprises determining an aryl hydrocarbon receptor (AHR) activation signature as disclosed in WO2020/201825, or AHR nuclear translocation, or the activity of cytochrome P-450 enzymes or the binding of AHR-ARNT to dioxin-responsive elements (DRE) using reporter assays.

Item 13. The method according to any one of items 1 to 12 wherein said biological sample is selected from a suitable sample comprising biological fluids, mammalian, for example human, cells, tissues, whole blood, cell lines, cellular supernatants, primary cells, IPSCs, hybridomas, recombinant cells, stem cells, and cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cells or tissues, and in particular cancer cells that do not derive from the immune system.

Item 14. The method according to any one of items 1 to 13, wherein said control sample is selected for example from a sample from a healthy subject or group of healthy subjects, a prior sample from the same patient, or a different patient or a patient group.

Item 15. The method according to any one of items 1 to 14, wherein said cancer is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells.

Item 16. The method according to any one of items 1 to 15, further comprising the step of a stratification of said patient to a disease and/or treatment group, for example a treatment group receiving suitable IL4I1 inhibitors.

Item 17. A diagnostic kit comprising materials for performing a method according to any one of items 1 to 16 in one or separate containers, optionally together with auxiliary agents and/or instructions for performing said method.

Item 18. A method of stratifying cancer patients into a high survival group and a low survival group comprising detecting the activity and/or expression of IL4I1 in a sample obtained from said cancer patients, wherein if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the low survival group and wherein if said expression or enzymatic activity of said IL4I1 is unchanged or decreased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the high survival group.

Item 19. A method of stratifying cancer patients according to item 18 wherein said increase of said activity and/or expression of said IL4I1 is detected in response to an immune therapy in said patient.

Item 20. The method according to any of the items 18-19, wherein patients in the high survival group are immune therapy responders and patients in the low survival group are immune therapy non-responders, in particular immune checkpoint blockade (ICB) non responders and/or IDO1 inhibitors non-responders.

Item 21. A method of stratifying cancer patients into responders and non-responders group to immune therapy comprising detecting the activity and/or expression of IL4I1 in a sample obtained from said cancer patients after said patients are subject to immune therapy, wherein if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the non-responders group and wherein if said expression or enzymatic activity of said IL4I1 is unchanged or decreased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the responders group.

Item 22. A method of stratifying cancer patients into responders and non-responders group to immune therapy according to item 21 wherein the control sample is from the same patients prior to being subject to said immune therapy.

Item 23. A method of stratifying cancer patients according to any of the items 18 to 22 wherein the method further comprises detecting the activity and/or expression of AHR in a sample obtained from said patient, wherein said activity and/or expression of AHR is modulated, preferably increased, when compared to a control.

Item 24. A method of stratifying cancer patients according to item 23 wherein said modulation of said activity and/or expression of said AHR is detected in response to immune therapy in said patient.

Item 25. A method of stratifying cancer patients according to any of items 19-24 wherein the immune therapy comprises immune checkpoint blockade (ICB) and/or IDO1 inhibitors.

Item 26. A method of treating a cancer in a subject in need thereof wherein the method comprises detecting the activity and/or expression of IL4I1 in a sample obtained from said patient, and wherein at least one IL4I1 inhibitor and/or at least one AHR modulator is administered in an effective amount to said subject if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group.

Item 27. A method of treating a cancer according to item 26 wherein the method further comprises detecting the activity and/or expression of AHR in a sample obtained from said patient, wherein said activity and/or expression of AHR is modulated, preferably increased, when compared to a control.

Item 28. The method of treating a cancer according to item 26 or 27 wherein said increase of said activity and/or expression of said IL4I1 and/or said modulation of the activity and/or expression of AHR is detected in response to an immune therapy in said patient.

Item 29. The method of treating a cancer according to any of item 26 to 28 comprising an immune therapy.

Item 30. The method of treating a cancer according to any of item 26 to 29, wherein the immune therapy comprises the use of at least one immune checkpoint inhibitor.

Item 31. The method of treating a cancer according to item 30 comprising an anti-CTLA4, anti-PD-L1, anti-PD1, anti-TIM3, anti-TIGIT, anti-LAG3 or a combination thereof.

Item 32. The method of treating a cancer according to any of item 28 or 31 wherein the immune therapy comprises a combination of immune therapy with inhibitors of IDO1, inhibitors of TDO2, AHR modulators and/or any other cancer treatments.

Item 33. A method of treating a cancer according to any of items 26 to 32, wherein the IL4I1 inhibitor is selected from 3-phenyl-2-piperidin-1-ylpropanoic acid, 2-(4-methylpiperazin-1-yl)-3-phenylpropanoic acid, 2-(diethylamino)-3-phenylpropanoic acid, 3-(2,6-dichlorophenyl)-2-piperidin-1-ylpropanoic acid, 3-phenyl-2-(propylamino)propanoic acid, 2-anilino-3-phenylpropanoic acid, L-Phenylalanine N-2-propen-1-yl-3-(trifluoromethyl), L-Phenylalanine, 4-cyano-N-phenyl, N-Propyl-L-phenylalanine, N-Phenyl-L-phenylalanine, 2-amino-3-phenyl-propionic acid. ethyl ester, 2-acteylamino-3-phenyl-propionic acid and 3-(2-pyridyl)-alanine.

Item 34. A method of treating a cancer according to any of items 26 to 33, wherein the modulators of AHR is selected from a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2, 1,3-dichloro-5-[(1E)-2-(4-methoxyphenyl)ethenyl]-benzene, α-Naphthoflavone, 6, 2′,4′-Trimethoxyflavone, CH223191, a tetrahydropyridopyrimidine derivative, StemRegenin-1, CH223191, GNF351, CB7993113 HP163, PX-A590, PX-A548, PX-A275, PX-A758, PX-A446, PX-A24590, PX-A25548, PX-A25275, PX-A25758, PX-A26446, an Indole AHR inhibitor, and an oxazole-containing (OxC) compound.

Item 35. A method of treating a cancer according to any one of items 26 to 34, wherein said cancer is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells.

Item 36. Inhibitors of IL4I1 for use in a method of treatment of a cancer in a patient identified as having an expression or enzymatic activity of IL4I1 increased in said patient when compared to control genes e.g., one or more housekeeping genes in said sample, or a control sample, e.g. a sample derived from a healthy subject, a group of healthy subjects, a prior sample from the same patient, a different patient or patient group.

Item 37. Inhibitors of IL4I1 for use in a method of treating a cancer according to item 36 wherein the method further comprises detecting the activity and/or expression of AHR in a sample obtained from said patient, wherein said activity and/or expression of AHR is modulated, preferably increased, when compared to a control.

Item 38. Inhibitors of IL4I1 for use in a method of treating a cancer according to item 37 wherein said increase of said activity and/or expression of said IL4I1 and/or said modulation of the activity and/or expression of AHR is detected in response to an immune therapy in said patient.

39. Inhibitors of IL4I1 for use in a method of treating a cancer according to any of item 36 to 38 comprising an immune therapy.

Item 40. Inhibitors of IL4I1 for use in a method of treating a cancer according to any of item 36 to 39, wherein the immune therapy comprises the use of at least an immune checkpoint inhibitor.

Item 41. Inhibitors of IL4I1 for use in a method of treating a cancer according to item 40 comprising an anti-CTLA4, anti-PD-L1, anti-PD1, anti-TB/13, anti-TIGIT, anti-LAG3 or a combination thereof.

Item 42. Inhibitors of IL4I1 for use in a method of treating a cancer according to any of item 36 to 41 the immune therapy comprises a combination of immune therapy with inhibitors of IDO1, inhibitors of TDO2, AHR modulators and/or any other cancer treatments.

Item 43. Inhibitors of IL4I1 for use in a method of treating a cancer according to any of items 36 to 42, wherein the IL4I1 inhibitor is selected from 3-phenyl-2-piperidin-1-ylpropanoic acid, 2-(4-methylpiperazin-1-yl)-3-phenylpropanoic acid, 2-(diethylamino)-3-phenylpropanoic acid, 3-(2,6-dichlorophenyl)-2-piperidin-1-ylpropanoic acid, 3-phenyl-2-(propylamino)propanoic acid, 2-anilino-3-phenylpropanoic acid, L-Phenylalanine N-2-propen-1-yl-3-(trifluoromethyl), L-Phenylalanine, 4-cyano-N-phenyl, N-Propyl-L-phenylalanine, N-Phenyl-L-phenylalanine, 2-amino-3-phenyl-propionic acid. ethyl ester, 2-acteylamino-3-phenyl-propionic acid and 3-(2-pyridyl)-alanine.

Item 44. Inhibitors of IL4I1 for use in a method of treating a cancer according to any one of items 36 to 43, wherein said cancer is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells.

Item 45. Modulators of AHR, in particular inhibitors of AHR for use in a method of treatment of a cancer in a patient identified as having an expression or enzymatic activity of IL4I1 increased in said patient when compared to control genes e.g., one or more housekeeping genes in said sample, or a control sample, e.g. a sample derived from a healthy subject, a group of healthy subjects, a prior sample from the same patient, a different patient or patient group.

Item 46. Modulators of AHR, in particular inhibitors of AHR for use in a method of treating a cancer according to item 45 wherein the method further comprises detecting the activity and/or expression of AHR in a sample obtained from said patient, wherein said activity and/or expression of AHR is modulated, preferably increased, when compared to a control.

Item 47. Modulators of AHR, in particular inhibitors of AHR for use in a method of treating a cancer according to item 46 wherein said increase of said activity and/or expression of said IL4I1 and/or said modulation of the activity and/or expression of AHR is detected in response to an immune therapy in said patient.

Item 48. Modulators of AHR, in particular inhibitors of AHR for use in a method of treating a cancer according to any of item 45 to 47 comprising an immune therapy.

Item 49. Modulators of AHR, in particular inhibitors of AHR for use in a method of treating a cancer according to any of item 45 to 48, wherein the immune therapy comprises the use of at least an immune checkpoint inhibitor.

Item 50. Modulators of AHR, in particular inhibitors of AHR for use in a method of treating a cancer according to item 49 comprising an anti-CTLA4, anti-PD-L1, anti-PD1, anti-TB/13, anti-TIGIT, anti-LAG3 or a combination thereof.

Item 51. Modulators of AHR, in particular inhibitors of AHR for use in a method of treating a cancer according to any of item 45 to 50 the immune therapy comprises a combination of immune therapy with inhibitors of IDO1, inhibitors of TDO2, AHR modulators and/or any other cancer treatments.

Item 52. Modulators of AHR, in particular inhibitors of AHR for use in a method of treating a cancer according to any of items 45 to 51, wherein the modulators of AHR is selected from a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2, 1,3-dichloro-5-[(1E)-2-(4-methoxyphenyl)ethenyl]-benzene, α-Naphthoflavone, 6, 2′,4′-Trimethoxyflavone, CH223191, a tetrahydropyridopyrimidine derivative, StemRegenin-1, CH223191, GNF351, CB7993113 HP163, PX-A590, PX-A548, PX-A275, PX-A758, PX-A446, PX-A24590, PX-A25548, PX-A25275, PX-A25758, PX-A26446, an Indole AHR inhibitor, and an oxazole-containing (OxC) compound.

Item 53. Modulators of AHR, in particular inhibitors of AHR for use in a method of treating a cancer according to any one of items 45 to 52, wherein said cancer is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells.

TABLE 1
Amino acids and metabolites converted and modulated by IL4I1

| | | PubChem |
|---|---|---|
| Chemical Name | InChI Key | CID |
| 3-Phenylpyruvic | BTNMPGBKDVTSJY- | 997 |
| acid (PP) | UHFFFAOYSA-N | |
| | InChI = 1S/C9H8O3/c10- | |
| | 8(9(11)12)6-7-4-2-1-3-5-7/ | |
| | h1-5H,6H2,(H,11,12) | |
| Indole-3-pyruvic acid | RSTKLPZEZYGQPY- | 803 |
| (I3P) | UHFFFAOYSA-N | |
| | InChI = 1S/C11H9NO3/c13- | |
| | 10(11(14)15)5-7-6-12-9- | |
| | 4-2-1-3-8(7)9/h1-4,6,12H, | |
| | 5H2,(H,14,15) | |
| 4- | KKADPXVIOXHVKN- | 979 |
| Hydroxyphenylpyruvic | UHFFFAOYSA-N | |
| acid (HPP) | InChI = 1S/C9H8O4/c10-7-3- | |
| | 1-6(2-4-7)5-8(11)9(12)13/h1- | |
| | 4,10H,5H2,(H,12,13) | |
| 2-Phenylacetic acid | WLJVXDMOQOGPHL- | 999 |
| | UHFFFAOYSA-N | |
| | InChI = 1S/C8H8O2/c9- | |
| | 8(10)6-7-4-2-1-3-5-7/h1- | |
| | 5H,6H2,(H,9,10) | |
| 4- | RGHHSNMVTDWUBI- | 126 |
| Hydroxybenzaldehyde | UHFFFAOYSA-N | |
| | InChI = 1S/C7H6O2/c8-5- | |
| | 6-1-3-7(9)4-2-6/h1-5,9H | |
| 2-Hydroxy-2- | IWYDHOAUDWTVEP- | 1292 |
| phenylacetic acid | UHFFFAOYSA-N | |
| | InChI = 1S/C8H8O3/c9- | |
| | 7(8(10)11)6-4-2-1-3-5-6/h1- | |
| | 5,7,9H,(H,10,11) | |
| 3-Indoleacetic | SEOVTRFCIGRIMH- | 802 |
| acid (IAA) | UHFFFAOYSA-N | |
| | InChI = 1S/C10H9NO2/c12- | |
| | 10(13)5-7-6-11-9-4-2-1-3- | |
| | 8(7)9/h1-4,6,11H,5H2, | |
| | (H,12,13) | |
| 1H-Indole-3-aldehyde | OLNJUISKUQQNIM- | 10256 |
| (I3A) | UHFFFAOYSA-N | |
| | InChI = 1S/C9H7NO/c11- | |
| | 6-7-5-10-9-4-2-1-3-8(7)9/h1- | |
| | 6,10H | |
| Indole-3-lactic | XGILAAMKEQUXLS- | 92904 |
| acid (ILA) | UHFFFAOYSA-N | |
| | InChI = 1S/C11H11NO3/ | |
| | c13-10(11(14)15)5-7-6-12- | |
| | 9-4-2-1-3-8(7)9/h1-4,6,10, | |
| | 12-13H,5H2,(H,14,15) | |
| 4-Hydroxyquinoline- | HCZHHEIFKROPDY- | 3845 |
| 2-carboxylic acid, | UHFFFAOYSA-N | |
| kynurenic acid | InChI = 1S/C10H7NO3/c12- | |
| (KynA) | 9-5-8(10(13)14)11-7-4-2-1-3- | |
| | 6(7)9/h1-5H,(H,11,12) | |
| | (H,13,14) | |
| 1,3-di(1H-indol-3- | OULRFLUQRMGBEN- | 11483104 |
| yl)acetone | UHFFFAOYSA-N | |
| | InChI = 1S/C19H16N2O/ | |
| | c22-15(9-13-11-20-18-7- | |
| | 3-1-5-16(13)18)10-14-12- | |
| | 21-19-8-4-2-6-17(14)19/h1- | |
| | 8,11-12,20-21H,9-10H2 | |
| (3Z)-1-(1H-indol- | BHYVQPWGWDXGBR- | 57345798 |
| 3-yl)-3-indol-3- | UKTHLTGXSA-N | |
| ylidenepropan-2-one | InChI = 1S/C19H14N2O/c22- | |
| | 15(9-13-11-20-18-7-3-1-5- | |
| | 16(13)18)10-14-12-21-19-8- | |
| | 4-2-6-17(14)19/h1-9,11- | |
| | 12,21H,10H2/b13-9+ | |
| L-valine | KZSNJWFQEVHDMF- | 6287 |
| | BYPYZUCNSA-N | |
| | InChI = 1S/C5H11NO2/c1- | |
| | 3(2)4(6)5(7)8/h3-4H,6H2,1- | |
| | 2H3,(H,7,8)/t4-/m0/s1 | |
| L-isoleucine | AGPKZVBTJJNPAG- | 6306 |
| | WHFBIAKZSA-N | |
| | InChI = 1S/C6H13NO2/c1-3- | |
| | 4(2)5(7)6(8)9/h4-5H,3,7H2,1- | |
| | 2H3,(H,8,9)/t4-,5-/m0/s1 | |
| L-leucine | ROHFNLRQFUQHCH- | 6106 |
| | YFKPBYRVSA-N | |
| | InChI = 1S/C6H13NO2/c1- | |
| | 4(2)3-5(7)6(8)9/h4-5H,3,7H2, | |
| | 1-2H3,(H,8,9)/t5-/m0/s1 | |
| L-alanine | QNAYBMKLOCPYGJ- | 5950 |
| | REOHCLBHSA-N | |
| | InChI = 1S/C3H7NO2/c1- | |
| | 2(4)3(5)6/h2H,4H2,1H3, | |
| | (H,5,6)/t2-/m0/s1 | |
| L-glutamic acid | WHUUTDBJXJRKMK- | 33032 |
| | VKHMYHEASA-N | |
| | InChI = 1S/C5H9NO4/c6- | |
| | 3(5(9)10)1-2-4(7)8/h3H,1- | |
| | 2,6H2,(H,7,8)(H,9,10)/t3-/ | |
| | m0/s1 | |
| L-methionine | FFEARJCKVFRZRR- | 6137 |
| | BYPYZUCNSA-N | |
| | InChI = 1S/C5H11NO2S/c1- | |
| | 9-3-2-4(6)5(7)8/h4H,2- | |
| | 3,6H2,1H3,(H,7,8)/t4-/m0/s1 | |
| L-glutamine | ZDXPYRJPNDTMRX- | 5961 |
| | VKHMYHEASA-N | |
| | InChI = 1S/C5H10N2O3/c6- | |
| | 3(5(9)10)1-2-4(7)8/h3H,1- | |
| | 2,6H2,(H2,7,8)(H,9,10)/t3-/ | |
| | m0/s1 | |
| 4-methylsulfanyl-2- | SXFSQZDSUWACKX- | 4584184 |
| oxobutanoate | UHFFFAOYSA-M | |
| | InChI = 1S/C5H8O3S/c1-9- | |
| | 3-2-4(6)5(7)8/h2-3H2,1H3, | |
| | (H,7,8)/p-1 | |
| Alpha-Keto-isoleucine | JVQYSWDUAOAHFM- | 439286 |
| | BYPYZUCNSA-N | |
| | InChI = 1S/C6H10O3/c1-3- | |
| | 4(2)5(7)6(8)9/h4H,3H2,1- | |
| | 2H3,(H,8,9)/t4-/m0/s1 | |
| alpha-Ketoisovalerate | QHKABHOOEWYVLI- | 49 |
| | UHFFFAOYSA-N | |
| | InChI = 1S/C5H8O3/c1- | |
| | 3(2)4(6)5(7)8/h3H,1-2H3, | |
| | (H,7,8) | |
| alpha-Ketoisocaproic | BKAJNAXTPSGJCU- | 70 |
| acid | UHFFFAOYSA-N | |
| | InChI = 1S/C6H10O3/c1- | |
| | 4(2)3-5(7)6(8)9/h4H,3H2,1- | |
| | 2H3,(H,8,9) | |
| L-proline | ONIBWKKTOPOVIA- | 145742 |
| | BYPYZUCNSA-N | |
| | InChI = 1S/C5H9NO2/c7- | |
| | 5(8)4-2-1-3-6-4/h4,6H,1- | |
| | 3H2,(H,7,8)/t4-/m0/s1 | |
| alpha-ketoglutaric acid | KPGXRSRHYNQIFN- | 51 |
| | UHFFFAOYSA-N | |
| | InChI = 1S/C5H6O5/c6- | |
| | 3(5(9)10)1-2-4(7)8/h1- | |
| | 2H2,(H,7,8)(H,9,10) | |
| (2Z)-3H-indol-3- | ZNWKELRZJMEGNL- | n.a. |
| ylidene-ethanoic acid | FNORWQNLBJ | |
| (oxidized indole-3- | InChI = 1/C10H7NO2/c12- | |
| acetic acid) | 10(13)5-7-6-11-9-4-2-1-3- | |
| | 8(7)9/h1-6H,(H,12,13) | |
| Indole-3-carboxylic | KMAKOBLIOCQGJP- | 69867 |
| acid | UHFFFAOYAT | |
| | InChI = 1/C9H7NO2/c11- | |
| | 9(12)7-5-10-8-4-2-1-3- | |
| | 6(7)8/h1-5,10H,(H,11,12) | |
| 4-Hydroxyphenyllactic | JVGVDSSUAVXRDY- | 9378 |
| acid | UHFFFAOYAV | |
| | InChI = 1/C9H10O4/c10-7-3- | |
| | 1-6(2-4-7)5-8(11)9(12)13/h1- | |
| | 4,8,10-11H,5H2,(H,12,13) | |
| Indole-3-carbinol | IVYPNXXAYMYVSP- | 3712 |
| | UHFFFAOYAO | |
| | InChI = 1/C9H9NO/c11-6-7- | |
| | 5-10-9-4-2-1-3-8(7)9/h1- | |
| | 5,10-11H,6H2 | |
| Phenyllactic acid | VOXXWSYKYCBWHO- | 3848 |
| | UHFFFAOYAI | |
| | InChI = 1/C9H10O3/c10- | |
| | 8(9(11)12)6-7-4-2-1-3-5-7/ | |
| | h1-5,8,10H,6H2,(H,11,12) | |

TABLE 2
Age adjusted multivariate cox proportional hazards of high expression
of IL4I1, IDO1 or TDO2 in GBM and LGG TCGA cohorts

| | | | Cox | | | |
|---|---|---|---|---|---|---|
| | | | proportional | | | |
| Tumor | Enzyme | coef | hazard | se. coef. | z | P-value |
| GBM | IL4I1 | 0.8247 | 2.281 | 0.206 | 3.999 | 6.391E−05 |
| GBM | IDO1 | 0.1501 | 1.162 | 0.184 | 0.816 | 0.414 |
| GBM | TDO2 | 0.4113 | 1.509 | 0.202 | 2.040 | 0.041 |
| LGG | IL4I1 | 1.2068 | 3.343 | 0.201 | 6.017 | 1.777E−09 |
| LGG | IDO1 | 0.8895 | 2.434 | 0.193 | 4.600 | 4.219E−06 |
| LGG | TDO2 | 0.4982 | 1.646 | 0.250 | 1.991 | 0.046 |

The invention shall now be further described in the following examples with reference to the accompanying figures, nevertheless, without being limited thereto. For the purposes of the present invention, all references as cited herein are incorporated by reference in their entireties.

As literal support for the priority right and for convenience, the items of the priority application are repeated below:

Item 1. A method for detecting and/or diagnosing cancer in a patient comprising detecting the expression or enzymatic activity of IL4I1 in a sample obtained from said patient, and diagnosing and/or detecting cancer in said patient, if said expression or enzymatic activity of said IL4I1 is increased in said patient, when compared to control genes e.g., one or more housekeeping genes in said sample, or a control sample, e.g. a sample derived from a healthy subject, a group of healthy subjects, a prior sample from the same patient, a different patient or patient group.

Item 2. The method according to item 1, wherein said detection of expression and/or enzymatic activity of IL4I1 in said sample comprises the detection of the amount and/or concentration of IL4I1 and/or IL4I1 metabolites in said sample.

Item 3. The method according to item 2, wherein said metabolites are selected from metabolites derived from the conversion of IL4I1 of phenylalanine, tyrosine and/or tryptophan, such as, for example, phenylpyruvic acid (PP), hydroxyphenylpyruvic acid (HPP), indole-3-pyruvic acid (I3P), 2-phenylacetic acid, phenyllactic acid, 4-hydroxybenzaldehyde, 2-hydroxy-2-phenylacetic acid, 4-hydroxyphenyllactic acid, and in particular the I3P derivatives indole-3-acetic acid (IAA), indole-3-aldehyde (I3A) and indole-3-lactic acid (ILA), 4-hydroxyquinoline-2-carboxylic acid (KynA), 1,3-di(1H-indole-3-yl)acetone, (3Z)-1-(1H-indole-3-yl)-3-indole-3-ylidenepropan-2-one, indole-3-carboxylic acid, oxidized indole-3-acetic acid, and indole-3-carbinol and/or the amino acids or amino acid metabolites L-valine, L-isoleucine, L-leucine, L-alanine, L-glutamic acid, L-methionine, L-glutamine, 4-methylsulfanyl-2-oxobutanoate, alpha-keto-isoleucine, alpha-ketoisovalerate, alpha-ketoisocaproic acid, L-proline, and alpha-ketoglutaric acid and any of the above in combination with ammonia and/or H₂O₂.

Item 4. The method according to any one of items 1 to 3, wherein detecting the expression and/or enzymatic activity of IL4I1 comprises the use of chromatography, NMR, metabolite sensors, antibodies, ELISAs, enzymatic assays, colorimetric assays, fluorescence assays, and/or H₂O₂ detection or ammonia detection, and/or detecting expression using genetic tools, such as chip analysis and primers and probes and PCR analysis, or detecting the amount of protein of IL4I1 using, for examples, antibodies, in samples, like biological fluids and cells/tissue samples.

Item 5. A method for detecting increased tumor cell motility in a cancer patient comprising detecting the activity and/or expression of IL4I1 in a sample obtained from said patient, and detecting increased tumor cell motility in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group.

Item 6. The method according to item 5, wherein said cancer is characterized by a modulation, such as an increase of the activity and/or expression of AHR, and/or is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM), preferably displaying IL4I1-expressing cells, and any relapsing and metastasizing forms thereof.

Item 7. A method for predicting or detecting or monitoring the effect of cancer immunotherapy in a cancer patient comprising detecting the activity and/or expression of IL4I1 in a sample obtained from said patient, wherein if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for and/or indicates a reduced effect and/or immune evasion of said cancer immunotherapy in said patient.

Item 8. The method according to item 7, wherein said increase of said activity and/or expression of said IL4I1 is detected in response to an immunotherapy in said patient.

Item 9. A method for detecting resistance against a cancer treatment comprising immunotherapy, such as for example immune checkpoint blockade (ICB), in a patient comprising detecting the activity and/or expression of IL4I1 in a sample obtained from said patient, and detecting resistance against a cancer treatment comprising ICB in said patient, if said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group.

Item 10. The method according to item 9, wherein said ICB treatment comprises the use of IDO1 inhibitors and/or antibodies selected from anti-CTLA-4, anti-PD-1, anti-PDL-1, anti LAG3 and anti BTLA.

Item 11. A method for predicting survival in a cancer patient comprising detecting the activity and/or expression of IL4I1 in a sample obtained from said patient, wherein said expression or enzymatic activity of said IL4I1 is increased in said patient when compared to control genes e.g., one or more housekeeping genes in said sample, or a control sample, e.g. a sample derived from a healthy subject, a group of healthy subjects, a prior sample from the same patient, a different patient or patient group is predictive for a reduced survival in said patient.

Item 12. The method according to any one of items 1 to 11, wherein said detecting of said activity comprises detecting IL4I1 metabolites, in particular IL4I1 tryptophan metabolites such as, for example I3P or I3P derived metabolites. More preferably, the IL4I1 tryptophan metabolites are selected from KynA, indole-3-acetic acid (IAA), indole-3-aldehyde (I3A) and/or indole-3-lactic acid (ILA) and indole-3-carbinol.

Item 13. The method according to any one of items 1 to 12, wherein detecting the enzymatic activity of IL4I1 comprises the use of chromatography, NMR, metabolite sensors, antibodies, ELISAs, enzymatic assays, colorimetric assays, fluorescence assays, and/or H₂O₂ or ammonia detection, and/or detecting expression using genetic tools, such as chip analysis and primers and probes and PCR analysis, or detecting the amount of protein of IL4I1 using, for examples, antibodies, in samples, like biological fluids and cells/tissue samples.

Item 14. The method according to any one of items 1 to 13, wherein said detection of said activity comprises detecting the activity and/or expression of AHR, wherein an increase in said activity and/or expression of AHR when compared to a control is indicative for an increase of the activity of IL4I1.

Item 15. The method according to any one of items 1 to 14, wherein said biological sample is selected from a suitable sample comprising biological fluids, mammalian, for example human, cells, tissues, whole blood, cell lines, cellular supernatants, primary cells, IPSCs, hybridomas, recombinant cells, stem cells, and cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and the transformed counterparts of said cells or tissues, and in particular cancer cells that do not derive from the immune system. In a preferred embodiment, the biological sample is cancer cells or tumor cells

Item 16. The method according to any one of items 1 to 15, wherein said control sample comprises control genes e.g., one or more housekeeping genes in said sample, or is a control sample derived for example from a healthy subject or group of healthy subjects, a prior sample from the same patient, a different patient or patient group.

Item 17. The method according to any one of items 1 to 16, wherein said cancer is characterized by a modulation, such as an increase of the activity and/or expression of AHR, and/or is preferably selected from the group consisting of B cell lymphoid malignancies, such as follicular lymphoma, Hodgkin lymphoma, primary mediastinal B cell lymphoma, diffuse large B cell lymphoma, marginal zone lymphoma and chronic lymphoid leukemia, ovarian carcinomas, mesotheliomas, colon carcinomas, breast carcinomas, melanomas, glioblastomas, prostate cancer, endometrial cancer, and lung carcinomas, preferably displaying IL4I1-expressing cells, and relapsing and/or metastasizing forms thereof.

Item 18. The method according to any one of items 1 to 17, further comprising the step of a stratification of said patient to a disease and/or group. In a preferred embodiment, the stratification of said patient is to a high survival group and a low survival group. In a more preferred embodiment, the patients in the high survival group are immunotherapy responders and patients in the low survival group are immunotherapy non-responders, in particular immune checkpoint blockade (ICB) non responders. In more preferred embodiment, the low survival group or immunotherapy non-responders is the group receiving an effective amount of IL4I1 inhibitors in a method of treatment of cancer patient.

Item 19. A diagnostic kit comprising materials for performing a method according to any one of items 1 to 18 in one or separate containers, optionally together with auxiliary agents and/or instructions for performing said method.

Item 20. Use of a diagnostic kit according to item 19 for a method according to any one of items 1 to 19.

Item 21. A method for treating and/or preventing an IL4I1-related disease or condition in a cell, for example in a patient in need of said treatment, comprising performing a method according to any one of items 1 to 18, and providing a suitable treatment to said patient, wherein said treatment is based, at least in part, on the results of the method according to the present invention.

### DESCRIPTION OF THE FIGURES

FIG. 1 shows that a pan-tissue AHR signature reveals a strong association of IL4I1 with AHR activity. Spider plots show the incidence of the seven Trp-degrading enzymes in AHR-associated modules (AAMs), depicted by the shaded area. The dots on the axes have values of either 0 (inner circle—absent) or 1 (outer circle—present). The shaded area is formed by connecting the dots representative for each tumor type.

FIG. 2 shows that IL4I1 activates the AHR. (A) IL4I1 expression in two human glioblastoma (GBM) specimens. IL4I1 staining (left), HE staining (right). Magnification 20×, scale bar 100 μm. (B) Barcode plots showing the regulation of the AHR signature in shCtrl (top) and shAHR (bottom) U-87MG glioblastoma cells expressing ectopic IL4I1, cultured for 120 h (n=3). (C) mRNA expression of selected AHR target genes in shCtrl and shAHR U-87MG cells expressing ectopic IL4I1, relative to shCtrl U-87MG cells without IL4I1 expression (dashed line), cultured for 120 h (n=3 for EGR1, IL1B, MMP1; n=4 for all other genes). (D) Immunoblot (left) and quantification (right) of nuclear to cytoplasmic ratio of AHR protein expression in LN-229 cells upon 4 h treatment with supernatants of Ctrl and IL4I1-expressing U-251MG cells, cultured for 120 h (n=3). (E) TIPARP mRNA expression in U-87MG cells treated for 24 h with 1 μM SR1 and medium (M) or supernatants of Ctrl and IL4I1-expressing U-87MG cells, relative to U-87MG cells treated with medium containing DMSO (n=3). (F) mRNA expression of selected AHR target genes in CAS-1 cells. Left plot: RNA interference of IL4I1 (siIL4I1), relative to cells treated with siCtrl (dashed line), cultured for 72 h (n=3). Right plot: downregulation of IL4I1 using CRISPR/Cas9 (sgIL4I1), relative to cells treated with non-targeting control sgRNA (dashed line), cultured for 72 h (n=4). n represents the number of independent experiments. Data are presented as mean±S.E.M and were analyzed by two-tailed paired student's t-test (C-F). * P<0.05, ** P<0.01, *** P<0.001.

FIG. 3 shows that IL4I1 promotes AHR-mediated pro-tumor effects. (A, B) Representative images (left) and quantification (right) of the percentage of the area covered by Ctrl and IL4I1-expressing U-87MG (A) and U-251MG (B) cells in a wound healing assay at the indicated time points (n=3 for 6 h, 9 h and 12 h in U-87MG and 48 h and 96 h in U-251MG, n=4 for 12 h in U-87MG). (C) Representative images (left) and quantification (right) of the percentages of the area covered by Ctrl and ectopically IL4I1-expressing U-87MG cells in the absence or presence of AHR inhibition with 1 μM SR1, in a wound healing assay at the indicated time points (n=3). (D) Kaplan Meier curves of the overall survival outcome of TCGA GBM (The Cancer Genome Atlas-glioblastoma multiforme) patients when divided into groups of high and low expression of IL4I1, IDO1 and TDO2. The p-values represent the probability of the age adjusted cox proportional hazard when comparing the group of high expression to that of low expression of the enzymes. (E) Overall survival probability as described in D but for the TCGA LGG (The Cancer Genome Atlas-Lower Grade Glioma) patient cohort Data are presented as mean±S.E.M and were analyzed by two-tailed unpaired student's t-test (A, B, C). * P<0.05, ** P<0.01, **** P<0.0001.

FIG. 4 shows that indole-3-pyruvate is the key metabolite mediating IL4I1-driven AHR activity and motility. (A) Concentration of Phe, Tyr and Trp in supernatants of Ctrl and IL4I1-expressing U-87MG cells, cultured for 120 h (n=3). (B-C) Representative images (B) and quantification (C) of the percentage of the area covered by U-87MG cells treated with 25 μM I3P and 1 μM SR1, in a wound healing assay at the indicated time points (n=5). n values represent the number of independent experiments. Data are presented as mean±S.E.M and were analyzed by two-tailed unpaired student's t-test (C). * P<0.05, ** P<0.01, *** P<0.001.

FIG. 5 shows that indole-3-pyruvate gives rise to kynurenic acid and indole-derivatives. IL4I1 activity generates indole-metabolites and KynA. Relative abundance of Kyn, KynA, I3P, IAA, I3A and ILA in supernatants of Ctrl and IL4I1-expressing U-87MG cells (120 h) (n=6 for Kyn, KynA, I3P and IAA; n=5 for I3A and ILA). n values represent the numbers of independent experiments. Data are presented as mean±S.E.M and were analyzed by one sample t-test * P<0.05, ** P<0.01, *** P<0.001, **** P<0.0001. n.s., not significant; n.d., not detected.

FIG. 6 shows that IL4I1 activity generates metabolites derived from aromatic amino acids. (A) Relative abundance of PP, PAA, HPP, HBA and HPAA in supernatants of Ctrl and IL4I1-expressing U-87MG cells (120 h) (n=6 for PP and HPP; n=5 for PAA and HPAA; n=4 for HBA). (B) Relative abundance of PP, PAA, HPP, HBA and HPAA in supernatants of Ctrl and IL4I1-expressing U-251MG cells (120 h) (n=6 for PP, HPP, HBA; n=5 for PAA and HPAA). (C) Relative abundance of Kyn, KynA, I3P, IAA, I3A and ILA in supernatants of Ctrl and IL4I1-expressing U-251MG cells (120 h) (n=6 for Kyn, KynA, I3P, IAA, I3A; n=5 for ILA). (D) Relative abundance of 4-hydroxyphenyllactic acid, indole-3-carboxylic acid, oxidized indole-3-acetic acid, phenyllactic acid and indole-3-carbinol in concentrated supernatants of Ctrl and IL4I1-expressing U87-MG and U-251MG cells (120 h) (n=6) n values represent the numbers of independent experiments. Data are presented as mean±S.E.M and were analyzed by two-tailed paired student's t-test. * P<0.05, ** P<0.01, *** P<0.001, **** P<0.0001. n.s., not significant; n.d., not detected.

FIG. 7 shows that IL4I1 expression is increased in cancer and metastasis. (A) Heatmap of the median log 2 TPM of IDO1, IDO2, TDO2 and IL4I1 expression in the Genotype-Tissue Expression (GTEx) dataset comprising 30 non-diseased tissues. Empty cells denote that no expression was detected. Dot size correspond to the expression level, small dot denoting low expression and big dot denoting high expression levels. (B) Heatmap of the median log 2 TPM of the enzymes as in A of 32 TCGA tumors. (C) IL4I1 expression as log 2 counts per million (log 2 CPM) comparing primary with metastatic melanoma of the TCGA cohort (unpaired Wilcoxon rank test; ****=P<0.0001). (D) Barcode plot showing AHR activation in metastatic versus primary melanoma patients of the TCGA cohort. (E) IL4I1 activity in metastatic melanoma resected from four individual patients in the presence of 10 mM Phe. Data are mean±SD (n=3, technical replicates per patient). (F) Spider plot showing the incidence of gene ontologies enriched in AAMs containing IL4I1.

FIG. 8 shows that IL4I1 is a metabolic immune checkpoint. (A) Bar graph representation of the significant incidences of differentially regulated immune cells infiltrating high vs low IL4I1 expressing TCGA tumors. (B) Heatmap representation showing the significant log 2 fold change of immune cell infiltration in TCGA tumors with high vs low IL4I1 expression separated by median expression of IL4I1. (C) Fold change of IDO1, IL4I1 and TDO2 expression in advanced melanoma patients before and after receiving nivolumab (anti-PD-1 mAb) treatment (GSE91061). (D) Barcode plot of AHR activation after nivolumab treatment in advanced melanoma patients. (E) IL4I1 expression levels as log 2 CPM in advanced melanoma in patients either naïve to ipilimumab (top) or having received ipilimumab preceding nivolumab treatment (bottom); before nivolumab therapy (light gray) and after receiving nivolumab (dark gray). The p-value was estimated using a paired Wilcoxon sum ranked test. (F) IL4I1, IDO1 and checkpoint (CP) expression reported as z-scores in advanced melanoma in patients naïve to ipilimumab before (light gray) and after (dark gray) receiving nivolumab treatment, grouped according to treatment response as progressive disease (left), partial or complete response (PR/CR, middle) and stable disease (right Unless otherwise stated, n values represent independent experiments. Data are presented as mean±S.E.M and were analyzed by one-way ANOVA with Dunnett's multiple comparisons test. *** P<0.001,**** P<0.0001. n.s., not significant.

FIG. 9 shows that high IL4I1 expression associates with poor survival outcome in different TCGA tumors. Kaplan Meier curves of the overall survival outcome of patient samples of six TCGA tumors when divided into groups of high and low expression of IL4I1. The TCGA tumors from left to right, top to bottom, are kidney renal clear cell carcinoma (KIRC), kidney renal papillary cell carcinoma (KIRP), liver hepatocellular carcinoma (LIHC), lung squamous cell carcinoma (LUSC), pancreatic adenocarcinoma (PAAD), and uveal melanoma (UVM). The p-values represent the probability of the age adjusted cox proportional hazard when comparing the group of high expression to that of low expression of the enzymes.

FIG. 10 shows Hallmark gene-sets differentially regulated after Nivolumab therapy (anti-PD1) (Riaz et. al. (PMID: 29033130)). Divergent barplot representation showing the hallmark of cancer gene sets that are significantly differentially up- or downregulated in advanced melanoma patients after receiving the mono-clonal anti-PD1 antibody Nivolumab compared to before receiving the treatment. Gene-sets are ordered in descending order. Orange barplots represent upregulated hallmark gene-sets, and green barplots represent downregulated gene-sets. The length of the gene corresponds to the enrichment score equivalent to the −log 10 FDR corrected p-value.

FIG. 11 shows the transcription start site of IL4I1 showing binding motifs of transcription factors with chromatin accessibility. Schematic representation of regulatory elements in the human IL4I1 promoter site at the transcription start site (chr19:49,894,887-49,898,887; ENCODE 3 Nov. 2018). The Layered H3K4Me1 and Layered H3K27Ac tracks show where modification of histone proteins is suggestive sites of regulatory activity. The Layered H3K4Me3 track shows histone marks associated with the IL4I1 promoter. The DNase I Hypersensitivity track indicates accessible chromatin sites. The Transcription Factor ChIP tracks show DNA binding regions of transcription factors regulating IL4I1 transcription as assayed by chromatin immunoprecipitation with antibodies specific to the transcription factor followed by sequencing of the precipitated DNA (ChIP-seq). The main transcription factors regulating IL4I1 transcription are POLR2A, TBP, ARNT, IRF4, STAT1, STAT3, and TCF7. The analysis was performed using the UCSC cancer genomics browser (PMID: 25392408).

FIG. 12 shows IL4I1 expression strongly associates with Hallmark gene-sets of cell motility and immune response across 32 TCGA tumors. Bubble plot showing the Pearson correlation coefficient between IL4I1 expression and biological process activity (BPA) scores generated for the 50 hallmarks of cancer gene sets across 32 TCGA tumors. The strength of the correlation is defined the intensity of the color. The bigger the size of the circle the higher the significance (smaller p-values). Empty fields represent no significant correlation. The gene sets with a black border highlight gene-sets involved in cell motility and the gene sets with a red border highlight gene-sets representing the immune functions.

FIG. 13 shows IL4I1 expression correlates strongly with breast cancer immune suppressive markers (Azizi 2018 (PMID: 29961579)). Bubble plot shows the Pearson correlation coefficient between IL4I1 expression and the expression of immune suppressive markers identified from the single cell RNA-seq dataset of breast cancer T cells reported by Azizi et. al (2018) (PMID: 29961579). The strength of the correlation is defined the intensity of the color. The bigger the size of the circle the higher the significance (smaller p-values). Empty fields represent no significant correlation.

FIG. 14 shows IL4I1 expression correlates strongly with lung tissue exhaustion markers (Sazbo 2019 (PMID: 31624246)). Bubble plot showing the Pearson correlation coefficient between IL 41 I expression and the expression of T cell exhaustion markers identified from the single cell RNA-seq dataset of lung tissue T cells reported by Sazbo et. al (2019) (PMID: 31624246). The strength of the correlation is defined the intensity of the color. The bigger the size of the circle the higher the significance (smaller p-values). Empty fields represent no significant correlation.

FIG. 15 shows IL4I1 expression correlates strongly with breast cancer Treg markers (Plitas 2016 (PMID: 27851913)). Bubble plot showing the Pearson correlation coefficient between IL4I1 expression and the expression of Treg markers identified from the single cell RNA-seq dataset of breast cancer T cells reported by Plitas et. al (2016) (PMID: 27851913). The strength of the correlation is defined the intensity of the color. The bigger the size of the circle the higher the significance (smaller p-values). Empty fields represent no significant correlation.

FIG. 16 shows IL4I1 expression correlates strongly with lung tissue Treg markers (Sazbo 2019 (PMID: 31624246)). Bubble plot showing the Pearson correlation coefficient between IL4I1 expression and the expression of Treg markers identified from the single cell RNA-seq dataset of lung tissue T cells reported by Sazbo et. al (2019) (PMID: 31624246). The strength of the correlation is defined the intensity of the color. The bigger the size of the circle the higher the significance (smaller p-values). Empty fields represent no significant correlation.

FIG. 17 shows IL4I1 expression correlates strongly with breast cancer Treg markers (Azizi 2018 (PMID: 29961579)). Bubble plot showing the Pearson correlation coefficient between IL4I1 expression and the expression of Treg markers identified from the single cell RNA-seq dataset of breast cancer T cells reported by Azizi et. al (2018) (PMID: 29961579). The strength of the correlation is defined the intensity of the color. The bigger the size of the circle the higher the significance (smaller p-values). Empty fields represent no significant correlation.

FIG. 18 shows IL4I1 expression correlates strongly with colorectal cancer Treg markers from blood (Zhang 2019 (PMID: 31341169)). Bubble plot showing the Pearson correlation coefficient between IL4I1 expression and the expression of Treg markers identified from the single cell RNA-seq dataset of T cells sorted from the blood of colorectal cancer patients reported by Zhang et. al (2019) (PMID: 31341169). The strength of the correlation is defined the intensity of the color. The bigger the size of the circle the higher the significance (smaller p-values). Empty fields represent no significant correlation.

FIG. 19 shows colorectal cancer Treg markers infiltrating the tumors (Zhang 2019 (PMID: 31341169)). Bubble plot showing the Pearson correlation coefficient between IL4I1 expression and the expression of Treg markers identified from the single cell RNA-seq dataset of T cells infiltrating colorectal cancer tissues of colorectal cancer patients reported by Zhang et. al (2019) (PMID: 31341169). The strength of the correlation is defined the intensity of the color. The bigger the size of the circle the higher the significance (smaller p-values). Empty fields represent no significant correlation.

FIG. 20 shows higher IL4I1 expression in colorectal cancer Tregs infiltrating the tumors than in Tregs circulating the blood of the same patients (Zhang 2019 (PMID: 31341169)). Boxplot representation showing higher expression of IL4I1 in Tregs infiltrating the tumor tissue of colorectal cancer patients compared to Tregs circulating in the blood of the same patients. IL4I1 expression is normalized log 2 counts per million (log 2(cpm+1)).

FIG. 21 shows IL4I1 strongly associates with immunotherapy non-response markers (Sade Feldman (PMID: 30388456)). Bubble plot showing the Pearson correlation coefficient between IL 4 I1 expression and immunotherapy non-response markers identified from the single cell RNA-seq dataset of advanced melanoma patients receiving anti-PD1, anti-CTLA-4 or a combination therapy (anti-PD1 and anti-CTLA-4) reported by Sade-Feldman et. al (2018) (PMID: 30388456). The strength of the correlation is defined the intensity of the color. The bigger the size of the circle the higher the significance (smaller p-values). Empty fields represent no significant correlation.

FIG. 22 show IL4I1 expression is higher in patient groups with higher enrichment of immunotherapy non-responder Tregs across 32 TCGA tumors. Boxplot representation of IL4I1 expression in 32 TCGA tumors divided into groups according to the enrichment score of the immunotherapy non-responsive Treg (NR-Tregs) cell population, identified from the single cell RNA-seq dataset of advanced melanoma patients receiving anti-PD1, anti-CTLA-4 or a combination therapy (anti-PD1 and anti-CTLA-4) reported by Sade-Feldman et. al (2018) (PMID: 30388456). Blue boxplots represent patient groups of low NR-Treg enrichment, and the red boxplots represent patient groups of high NR-Treg enrichment. IL4I1 expression is normalized log 2 counts per million (log 2(cpm+1)).

### EXAMPLES

Material and Methods

Microarray and RNA-Seq Data Analysis

Array datasets-For the microarray datasets generated from the different experimental conditions in this study, the Affymetrix WT PLUS Reagent Kit was used to generate labeled ss-cDNA from input amounts of 100 ng total RNA. 5.5 μg of fragmented and labeled ss-cDNA were hybridized for 17 h at 45° C. on Affymetrix Human Gene 2.0 ST Arrays according to the manufacturer's instructions. Gene Expression Microarrays were scanned using the Affymetrix GeneChip® Scanner 3000 according to the GeneChip® Expression Wash, Stain and Scan Manual for Cartridge Arrays (P/N 702731). The Affymetrix microarray chips “human gene 2.0 ST” were annotated using NetAffx and analyzed using the oligo package (Carvalho et al., 2007). Other Affymetrix chips were analyzed using the affy and affycoretools packages (Gautier et al., 2004). Raw CEL files were imported from disk or downloaded from GEO using GEOquery (Davis and Meltzer, 2007), followed by RMA normalization and summary.

RNA-seq datasets-Raw counts and metadata were downloaded from GEO using GEOquery (Davis and Meltzer, 2007). The harmonized HT-Seq counts and Fragments Per Kilobase of transcript per Million mapped reads (FPKM) of TCGA datasets were downloaded using TCGAbiolinks (Colaprico et al., 2016) from Genomic Data Commons (GDC) (https://gdc.cancer.gov/), and only patients with the identifier “primary tumor” were retained, with the exception of melanoma that was split into datasets for primary and metastatic cohorts. The FPKM values were converted to transcripts per million (TPMs) (Li and Dewey, 2011). TPM data of normal tissues were downloaded from the Genotype-Tissue Expression (GTEx) dataset (https://gtexportal.org/home/). All TPM values were log 2 transformed. Raw counts of the Riaz et al. (2007) (Riaz et al., 2017) advanced melanoma dataset were downloaded from https://github.com/riazn/bms038_analysis, and based on principle component analysis one outlier patient pair was removed (Pt-84). RNA-seq counts were saved as DGELists (Robinson et al., 2010). Genes with less than 10 counts were filtered followed by trimmed mean of M values (TMM) normalization (Robinson and Oshlack, 2010) and variance modeling using voom (Law et al., 2014).

Colorectal Cancer scRNA-Seq Dataset

The scRNA-seq immune cells dataset of colorectal cancer (CRC) patients (Zhang et al., 2019) (PMID: 31341169) was retrieved from GEO (GSE108989) as a raw counts matrix. The dataset was converted as previously described for analysis using scanpy. Apoptotic cells were removed if the percentage of their mitochondrial gene expression was higher than 10%. Cells with more than 8000 genes were considered as doublets and were removed, leaving a total number of 10833 cells. The count matrix was normalized to counts per million (CPM+1), and scaled to the natural logarithm.

Highly variable genes were selected if they had a dispersion of at least 0.5 and a mean expression value of eight. The effect of mitochondrial genes as well as the number of counts per cell were regressed out using the function regress.out to correct for potential confounding effects between expression profiles due to cell stress and cell size. An additional weighted scaling was performed followed by scaling to unit variance. Genes with a unit variance higher than of 10 were removed.

Survival Analysis

Patients of the TCGA cohorts were grouped into high or low gene expression groups by estimating a Monte-Carlo based maximal selected ranked statistics (Hothorn and Zeileis, 2008). Survival difference between the groups was estimated by fitting an age adjusted cox proportional hazard model. Kaplan-Meier curves were used for visualizing the fitted cox proportional hazard models. The reported p-value is that of the proportional hazard ratio of the high versus low group comparison.

Defining Infiltrating Immune Populations and Estimating the Immunophenogram Scores

Metagenes describing 28 different immune populations were retrieved from Charoentong et al. (Charoentong et al., 2017). Sample-wise GSVA scores were generated for each tumor type. The difference between the infiltrating cells in the tumors separated by median IL4I1 expression (high vs low) was performed using the limma package (Ritchie et al., 2015). Only populations showing a log 2 fold change of at least 0.3 and an adjusted p-value of 0.05 were considered significant. The scores of the four compartments of the immunophenogram (antigen processing machinery (MHC), checkpoints (CP), effector cells (EC) and suppressor cells (SC)) were generated using the pipeline described by the authors of Charoentong et al. (Charoentong et al., 2017). The CP values generated are a negative weighted z-score, which corresponds to high expression of suppressive molecules and low expression of effector molecules. To allow easier comparison of CP values in the response groups before and after nivolumab treatment, the negative weighted z-scores were multiplied by −1. After this multiplication step, positive CP values will indicate high expression of suppressive molecules and low expression of effector molecules, which can be easily compared to expression levels of IL4I1 and IDO1 in the same groups. The CP molecules and their effect on immune response are reported in Table S8.

Gene Correlation Networks Associated with AHR Activity

The normalized DGELists of TCGA tumors were used for weighted gene co-expression network analysis (WGCNA) (Langfelder and Horvath, 2008). Soft thresholds were estimated for signed hybrid networks in single block settings. WGCNA was run using bi-weighted correlations and Eigen genes representing the first principle components of each module were returned. Selection of WGCNA modules associated with AHR activity was conducted by performing a global test (Goeman and Finos, 2012; Goeman et al., 2004) using the AHR activity as the response and the WGCNA modules as the model predictors. AHR activity was correlated with WGCNA modules using Pearson correlation. Modules that overlapped the global test and the Pearson correlation results, with a p-value of 0.05 or less in both tests, were selected as the AHR-associated modules (AAMs). Modules with both positive and negative associations with AHR activity were considered.

Biological Process Activity Scores

The hallmark gene-sets of cancer were downloaded from the MSigDb database (v6) (Liberzon et al., 2011), in addition, gene-sets describing Treg cell populations extracted from single cell data of infiltrating immune cells from breast cancer (PMID: 29961579; PMID: 27851913), lung tissue (PMID: 31624246) and colorectal cancer (PMID: 31341169) and blood of the colorectal cancer patients (PMID: 31341169). The gene-sets were used to estimate a normalized enrichment score for their respective biological processes (BPA score) (PMID: 31653878; PMID: 27322546) in the normalized bulk RNA-seq data of 32 TCGA tumors. The BPA scores were correlated with the expression level of IL4I1 across the 32 TCGA tumors using Pearson's correlation coefficient.

Correlation Analysis

The correlation between the expression of IL4I1 and markers of immune suppression, T cell exhaustion or Tregs in the 32 TCGA tumors was performed using Pearson's correlation coefficient. These markers were identified from single cell data of infiltrating immune cells from breast cancer (PMID: 29961579; PMID: 27851913), lung tissue (PMID: 31624246) and colorectal cancer (PMID: 31341169) and blood of the colorectal cancer patients (PMID: 31341169).

Evaluating the Enrichment of Immunotherapy Non-Responder Tregs in TCGA Tumors

Generating a single sample score for the immunotherapy non-responder Treg cell populations (NR-Treg) in the 32 TCGA tumors was performed by performing gene set variance analysis. This method transforms the expression matrix from a gene/sample matrix into a gene-set per sample matrix allowing the assessment of the activity of the underlying pathway. This method is both non-parametric and unsupervised and it bypasses the need for constructing a design or contrast matrix to model the conditions tested or the different phenotypes. One of the strengths of this method is allowing direct sample to sample comparisons, owed to interrogating the relative enrichment of pathways within a sample instead of absolute enrichment between conditions by focusing on searching the sample space instead of phenotypes.

To overcome the gene specific biases, such as GC content or gene length biases, the expression values are brought to a similar scale by a non-parametric kernel estimation of its cumulative density function performed using a Gaussian kernel. This estimated expression level statistic is ranked and ordered to produce a distribution of the genes that allows evaluating if the genes of a gene-set(s) are present on either tails of the rank distribution. Then a normalized enrichment score is estimated using a Kolmogorov Smirnov like random walk statistic. This method penalizes large deviations of the genes of the gene-set(s) at the tails of the distribution, therefore, if genes of a gene-set(s) are both up and down regulated, they will cancel each other out, emphasizing that positive or negative scores would reflect if genes of a gene-set(s) are up or down regulated. The resulting enrichment score is calculated as the difference between the magnitude of the largest positive and negative random walk deviations. Based on simulations of the null distribution, where no change in gene expression is present, a threshold of the normalized enrichment score greater than 0.1 would be used to indicate up- or downregulation.

Cell Culture

U-87MG were obtained from ATCC. CAS-1 and U-251MG were from ICLC and ECACC, respectively. CAS-1, HEK293T, LN-229, U-87MG and U-251MG were cultured in phenol red-free high glucose DMEM medium (Gibco, 31053028) supplemented with 10% FBS (Gibco, 10270106), 2 mM L-glutamine (Gibco, 25030-024), 1 mM sodium pyruvate (Gibco, 11360-039), 100 U/mL penicillin and 100 μg/mL streptomycin (Gibco, 15140-122) (henceforth, referred to as complete DMEM. Cell lines were cultured at 37° C. and 5% CO₂.

Generation of Transgenic Cell Lines

A human IL 4I1 cDNA clone flanked by Gateway compatible recombination sites was purchased from MyBiosource (MBS1270935). The cDNA clone was recombined into the lentivirus compatible Gateway expression vector pLX301 (a gift from D. Root, Addgene plasmid 25895) (Yang et al., 2011). Production of lentiviruses was achieved by transfecting HEK293T with pMD2.G (a gift from D. Trono, Addgene plasmid 12259), psPAX2 (a gift from D. Trono, Addgene plasmid 12260), and lentiviral plasmid, using FuGENE HD (Promega, E2311) according to the manufacturer's protocol. Viral supernatants were harvested at 48 h and 72 h, pooled and filtered through a 0.45 μm pore filter. Stable IL4I1-expressing (pLX301-IL4I1) and control (pLX301) cell lines were generated by infecting U-87MG and U-251MG cells for 24 h with the respective viral supernatants in presence of 8 μg/mL polybrene (Merck Millipore, TR-1003-G), followed by selection with medium containing 1 μg/mL puromycin (AppliChem, A2856). Stable expression of IL4I1 was confirmed by qRT-PCR, immunoblot and IL4I1 enzymatic activity.

Stable knockdown of AHR in U-87MG cells was achieved using shERWOOD UltramiR Lentiviral shRNA targeting AHR (transOMIC Technologies, TLHSU1400-196-GVO-TRI). Glioma cells were infected with viral supernatants containing either shAHR or shControl (shC) sequences to generate stable cell lines.

shERWOOD UltramiR shRNA sequences are:

shAHR (ULTRA-3234821):
(SEQ ID NO: 1)
5’-TGCTGTTGACAGTGAGCGCAGGAAGAATTGTTTTAGG
ATATAGTGAAGCCACAGATGTATATCCTAAAACAATTCTTCCTTTGCC
TACTGCCTCGGA-3’;
shC (ULTRA-NT#4):
(SEQ ID NO: 2)
5’-TGCTGTTGACAGTGA GCGAAGGCAG
AAGTATGCAAAGCATTAGTGAAGCCACAGATGTAATGCTTTGCA
TACTTCTGCCTGTGCCTACTGCCTCGG A-3′.

siRNA mediated gene knockdown of IL4I1 was carried out using ON-TARGETplus Human SMARTpool siRNA reagent (Dharmacon, L-008109-00-0005). siRNA transfections were done with Lipofectamine RNAiMAX (Thermo Fisher Scientific, 13778100), following the manufacturer's protocol. ON-TARGETplus Non-targeting Pool siRNA (Dharmacon, D-001810-10-05) was used as control.

For gene and protein expression experiments involving Ctrl and IL4I1-expressing U-87MG and U-251MG cells, 4×10⁵ cells per well were seeded in 2 mL in 6-well plates and incubated for 72 h or 120 h. For metabolomics experiments, cells were seeded at a density of 4×10⁵ cells per well in 2 mL of complete DMEM in 6-well plates and incubated for 24 h. In cases where more cells and supernatant were needed, 2.6×10⁶ cells per 10 cm dish were seeded in 13.3 mL of complete DMEM and incubated for 24 h. Afterwards, cells were washed once with phosphate-buffered saline (PBS), 2 or 13.3 mL of fresh FBS-free DMEM were added (depending on the well size and cell density used) and cells were incubated for 120 h. Supernatants were snap frozen in liquid nitrogen and stored at −80° C. until metabolite measurement.

Experiments targeting AHR with siRNA in U-87MG cells were achieved by seeding 4×10⁵ cells per well in 6-well plates, followed by incubation for 24 h. Cells were transfected with either siCtrl or siAHR and fresh medium containing treatment was added 48 h later. For experiments where AHR and IL 41 I were knocked down in CAS-1 cells, 4×10⁵ cells per well in 6-well plates were seeded and incubated for 24 h. Cells were transfected with respective siCtrl or targeting siRNA. Complete DMEM was replaced with 1.5 mL of FBS-free DMEM 24 h post-transfection and cells were incubated for 72 h.

CAS-1 cells with stable downregulation of IL4I1 were generated using transEDIT CRISPR All-in-one lentiviral expression vectors targeting IL4I1 (transOMIC Technologies, CAHS1001-259307-GVO-TRI). Cells were exposed to viral supernatants containing either sgControl (sgCtrl) or sgIL4I1 for 24 h, in presence of 8 μg/mL polybrene (Merck Millipore, TR-1003-G). Transduced cells were selected by sorting ZsGreen positive cells using a BD FACSAria Fusion (BD Biosciences). transEDIT CRISPR All-in-one sgRNA sequences without PAM (5′-3′) are:

sgCtrl (TELG1017):
(SEQ ID NO: 27)
GGAGCGCACCATCTTCTTCA
sgIL4I1-2 (TEVH-1143054):
(SEQ ID NO: 28)
GGGCCGCATCTTCACCTACC

Migration Assays

Cells were seeded in 2-well cell culture inserts (ibidi, 80209) placed in 24-well plates. For U-87MG cells, as well as Ctrl and IL4I1-expressing U-87MG cells, 3×10⁴ cells in 100 μL medium per well of cell culture insert were seeded. In the case of Ctrl and IL4I1-expressing U-251MG cells, 4×10⁴ in 100 μL medium per well of cell culture insert were seeded. Cells were incubated for 16 h, inserts were removed and 1 mL of complete DMEM medium was added to each well of the plate. Cell migration was monitored by taking pictures at indicated time points, until gap (500 μm+/−100 μm) was covered by cells. In the case where U-87MG cells were treated with I3P or SR1, treatments were applied at the time of seeding the cells in the 2 well cell culture inserts. In addition, after 16 h incubation time and insert removal, 1 mL of complete DMEM medium containing treatments was applied to cells and kept throughout the assay. For each independent experiment, 3 pictures per well were taken and each condition was carried out at least in 3 wells. Pictures were taken with an ECLIPSE Ti-E/B inverted microscope (Nikon) with the 4×objective and using the NIS Elements software version 4.13.04. Pictures were analyzed with TScratch software version 1.0 (Geback et al., 2009). Cell migration was assessed by calculating the percentage of covered area for each time point normalized to the area at the 0 h time point.

RNA Isolation and Real Time PCR

Total RNA was harvested from cultured cells using the RNeasy Mini Kit (Qiagen, 80204) followed by cDNA synthesis using the High Capacity cDNA reverse transcriptase kit (Applied Biosystems, 4368813). A StepOne Plus real-time PCR system (Applied Biosystems) was used to perform quantitative real-time PCR (qRT-PCR) of cDNA samples using SYBR Select Master mix (Thermo Scientific, 4309155). Data was processed and analyzed using the StepOne Software v2.3. Relative quantification of target genes was done against RNA18S as reference gene using the 2^{-ΔΔCt} method. Human primer sequences are listed in Table 3.

TABLE 3
Human primer sequences

| Gene | | |
|---|---|---|
| Name | Forward Primer (5′→3′) | Reverse Primer (5′→3′) |
| ABCG2 | TTCCACGATATGGATTTACGG | GTTTCCTGTTGCATTGAGTCC |
| | (SEQ ID NO: 3) | (SEQ ID NO: 4) |
| AHRR | CCCTCCTCAGGTGGTGTTTG | CGACAAATGAAGCAGCGTGT |
| | (SEQ ID NO: 5) | (SEQ ID NO: 6) |
| CYP1B1 | GACGCCTTTATCCTCTCTGCG | ACGACCTGATCCAATTCTGC |
| | (SEQ ID NO: 7) | C (SEQ ID NO: 8) |
| EGR1 | CTGACCGCAGAGTCTTTTCCT | GAGTGGTTTGGCTGGGGTAA |
| | (SEQ ID NO: 9) | (SEQ ID NO: 10) |
| EREG | CTGCCTGGGTTTCCATCTTCT | GCCATTCATGTCAGAGCTAC |
| | (SEQ ID NO: 11) | ACT (SEQ ID NO: 12) |
| IL1B | CTCGCCAGTGAAATGATGGCT | GTCGGAGATTCGTAGCTGGA |
| | (SEQ ID NO: 13) | T (SEQ ID NO: 14) |
| IL4I1 | CGCCCGAAGACATCTACCAG | GATATTCCAAGAGCGTGTGC |
| | (SEQ ID NO: 15) | C (SEQ ID NO: 16) |
| MMP1 | GCTAACCTTTGATGCTATAACTA | TTTGTGCGCATGTAGAATCT |
| | CGA (SEQ ID NO: 17) | G (SEQ ID NO: 18) |
| NPTX1 | CATCAATGACAAGGTGGCCAAG | GGGCTTGATGGGGTGATAGG |
| | (SEQ ID NO: 19) | (SEQ ID NO: 20) |
| RNA18S | GATGGGCGGCGGAAAATAG | GCGTGGATTCTGCATAATGG |
| | (SEQ ID NO: 21) | T (SEQ ID NO: 22) |
| SERPINB2 | ACCCCCATGACTCCAGAGAA | CTTGTGCCTGCAAAATCGCA |
| | (SEQ ID NO: 23) | T (SEQ ID NO: 24) |
| TIPARP | CACCCTCTAGCAATGTCAACTC | CAGACTCGGGATACTCTCTC |
| | (SEQ ID NO: 25) | C (SEQ ID NO: 26) |

Protein Isolation and Western Blots

For all experiments, whole cell lysates were prepared in ice cold tris(hydroxymethyl)aminomethane (281 mM; Merck, 1083870500), Tris HCl (212 mM; Carl Roth, 9090.4) and EDTA (1 mM, pH 8; Gibco, 15575-038) containing 4% sodium dodecyl sulfate (SDS; VWR, 442444H) and glycerol (40%, Sigma Aldrich) supplemented with cOmplete™ Protease Inhibitor Cocktail (Roche, 11697498001). For AHR translocation assays, protein content in the nuclear and the cytoplasmic fractions of LN-229 glioblastoma cells was compared by immunoblotting. LN-229 cells were treated with supernatants of Ctrl and IL4I1-expressing U-251MG cells (120 h) for 4 h. Lysates were snap frozen in liquid nitrogen and thawed three times following 10 cycles of ultrasonication after each freeze-thaw cycle. To isolate protein from the two different cellular fractions NE-PER™ Nuclear and Cytoplasmic Extraction Reagents (Thermo Fisher Scientific, 78835) were used. Extraction was performed following the manufacturer's instructions. Nuclear specific Lamin A served as control for appropriate fractionation and was detected using polyclonal rabbit anti-Lamin A antibody (BioLegend, 613501; 1:500). AHR was detected using the primary mouse monoclonal anti-AHR antibody clone RPT1 (Abcam, ab2770). After incubation with the primary antibody membranes were washed three times for 10 min and incubated with a 1:5000 dilution of horseradish peroxidase-conjugated anti-rabbit (for IL4I1, Lamin A; BioRad, 170-6515) or anti-mouse antibody (for β-actin, AHR; BioRad, 170-6516) for 1 h at RT, respectively. Antibodies were prepared in wash buffer (50 mM Tris-HCL, 50 mM Tris-Base, 150 mM NaCl, pH 6.8) containing 2% milk powder (Carl Roth, T145.2). Immunoblots were detected using ECL Select Western Blotting Detection Reagent (Amersham, RPN2235) on a FluorChem FC3 system (BioLabTec).

Metabolomic Analyses

Consumption of phenylalanine, tyrosine and tryptophan by control and IL4I1-expressing and non-expressing U-87MG and U-251MG cells was assessed by quantification of the amino acids in the cell culture supernatants after 120 h incubation. For phenylalanine and tyrosine detection, amino acids were labeled with the fluorescent dye AccQ-Tag™ (Waters) according to the manufacturer's protocol. The derivatization product was separated at 42° C. on an Acquity BEH C18 column (Waters) using an Acquity H-class UPLC system (Waters) coupled to an Acquity fluorescence detector (FLR) (Waters). Samples were analyzed on the UPLC as described by Yang et al. (2015) (Yang et al., 2015). For analyses of Trp consumption, supernatants were mixed with an equal volume of 12% perchloric acid and incubated on ice for 10 min. Prior to analysis, samples were centrifuged for 10 min at 4° C. and 16,400 g to precipitate proteins and remove remaining cell debris. Metabolites were then separated by reversed phase chromatography on an Acquity HSS T3 column (100 mm×2.1 mm, 1.7 μm, Waters) connected to an Acquity H-class UPLC system (Waters). The column was heated to 37° C. and equilibrated with 5 column volumes of 100% solvent A (20 mM sodium acetate, 3 mM zinc acetate, pH 6) at a flow rate of 0.55 mL/min. Clear separation of Trp was achieved by increasing the concentration of solvent B (acetonitrile) in solvent A as follows: 4 min 0% B, 10 min 5% B, 13 min 15% B, 15 min 25% B, and return to 0% B in 3 min. Trp was detected by fluorescence (Acquity FLR detector, Waters, excitation: 254 nm, emission: 401 nm). Standards were used for quantification (Sigma). Data acquisition and processing was performed with the Empower3 software suite (Waters).

Further, the inventors conducted an untargeted metabolomics approach to identify metabolites that were differentially abundant in supernatants of Ctrl and IL4I1-expressing U-87MG and U-251MG cells cultured for 120 h. To this end, 50 μL of cell culture supernatants were mixed with 200 μL ice-cold acetonitrile by vortexing followed by incubation at −20° C. for 1 h. Samples were centrifuged for 15 min at 4° C. and transferred into TruView UPLC-MS vials (Waters). A pool sample was prepared by mixing equal volumes of all samples. Samples were measured using an I-class UPLC system coupled to a Vion IMS QTof MS (Waters). Metabolites were separated using either a Cogent Diamond Hydride 2.0 column (150×2.1 mm, 2.2 μm; MicroSolv USA) or a HSS T3 column (100×2.1 mm, 1.8 μm; Waters). For instrument control and acquisition of MS data UNIFI 1.8.2 (Waters) was used. Follow-up data analyses were performed using Progenesis QI (Waters). ILA was detected at 204.0662 Da (neg. mode; expected monoisotopic mass: 205.0739 Da; deviation −2 ppm) and identified by the expected fragment ions at 116.0495, 128.0495, 130.0652 and 204.0655 Da. The Trp-, Phe- and Tyr-derived metabolites were identified by comparing their fragmentation patterns resulting from LC-MS measurements using an HPLC (Agilent 1290) coupled to a triple quad MS (Agilent 6460) with external standards. For targeted quantification of the metabolites, MRM mode was used. For all test compounds 10 mM stock solutions were prepared by gravimetrically adding the required amount into 1.5 mL Eppendorf safe-lock tubes and dissolving the test compound in 1 mL DMSO. For each compound, stock solutions covered a concentration range from 10 mM to 0.039 mM.

For quantification of the metabolites, 300 μL of each bioassay supernatant was added to Eppendorf safe-lock tubes. Associated calibration samples were prepared by adding 300 cell culture media and 1 μL of test compound stock solution into an Eppendorf safe-lock tube. Subsequently, 300 μL acetonitrile was added to trigger precipitation of media components. All samples were centrifuged at 8000 rpm for 4 min and 150 μL of the supernatants was transferred into 1.5 mL glass vials equipped with 200 μL glass inserts for HPLC analysis. 5 μL of the sample was injected for the analysis. Water and acetonitrile with 0.1% formic acid were used as eluent A and B, respectively for HPLC separation for 5 min with a flow rate of 0.5 mL/min. Separation was achieved using a 50 mm long Poroshell 120 EC-C18 2.7 micron column (Agilent). The Agilent Jetstream ESI source was set to gas with a gas sheath temperature of 300° C., a gas flow of 10 L/min and a sheath gas flow of 11 L/min. The nebulizer pressure was set to 55 psi and capillary voltage at 2000 V throughout the run. For instrument control and data acquisition MassHunter software suite (Agilent) was used.

Software and Statistics

Graphical and statistical analyses of qRT-PCR, protein, as well as metabolite data were performed using GraphPad Prism software version 8.0. Unless otherwise indicated, data represents the mean±S.E.M of at least 3 independent experiments. In cases where data was expressed as fold change, these values were log 10 transformed and the resulting values were used for statistical analysis. Depending on the data, the following statistical analyses were applied: one sample t-test, two-tailed student's t-test (paired or unpaired), one-way ANOVA with Tukey's multiple comparisons test and repeated measures ANOVA with Dunnett's multiple comparisons test. Significant differences were reported as * P<0.05, ** P<0.01, *** P<0.001, **** P<0.0001. n. s. indicates no significant difference. For bioinformatics analysis, unless stated otherwise, all pairwise comparisons were performed using Kruskal-Wallis and Wilcoxon sum rank tests, and all reported p-values were adjusted using the Benjamini-Hochberg procedure.

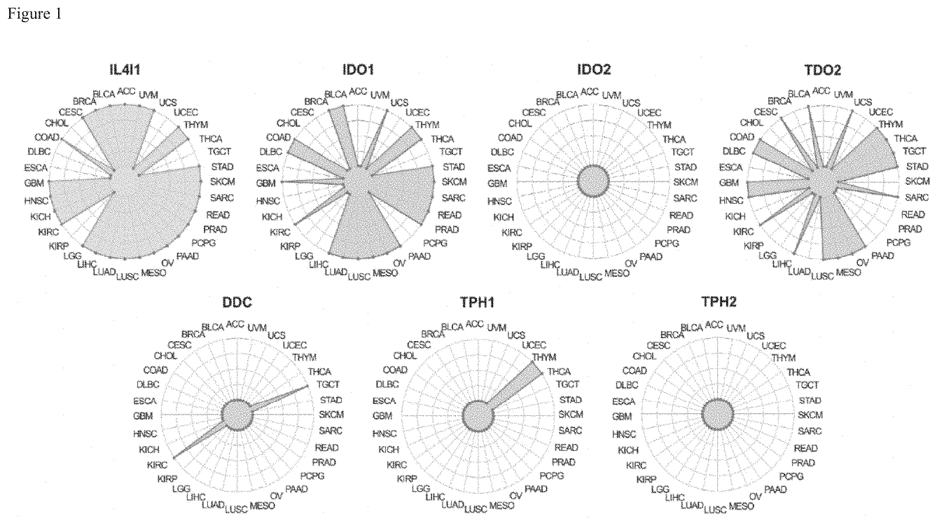

The L-amino acid oxidase IL4I1 exhibited the highest incidence in AHR associated modules (AAMs), as it was present in 26 out of the 32 tumor types (FIG. 1). Thus, AHR activity in human tumors associates more strongly with IL4I1 than with any other Trp-degrading enzyme, including the two bona fide AHR activators IDO1 and TDO2. This was surprising, as IL4I1 is mainly recognized for catalyzing the oxidative deamination of phenylalanine (Phe) to phenylpyruvate (PP), while producing hydrogen peroxide (H₂O₂) and ammonia (Boulland et al., 2007; Mason et al., 2004), but has not yet been implicated in AHR signaling.

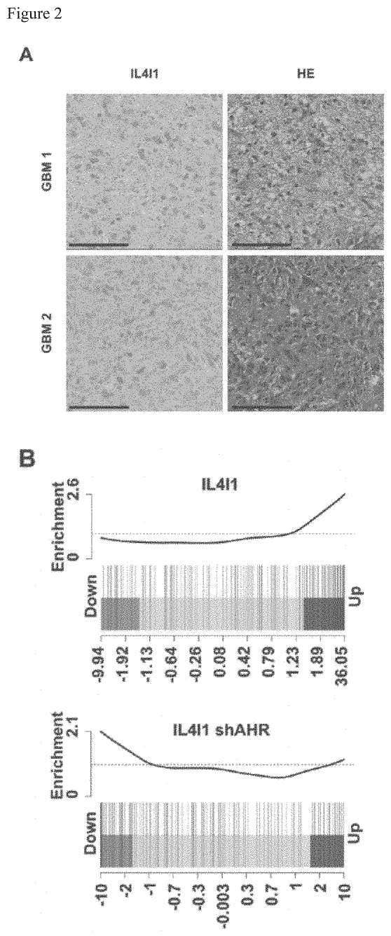

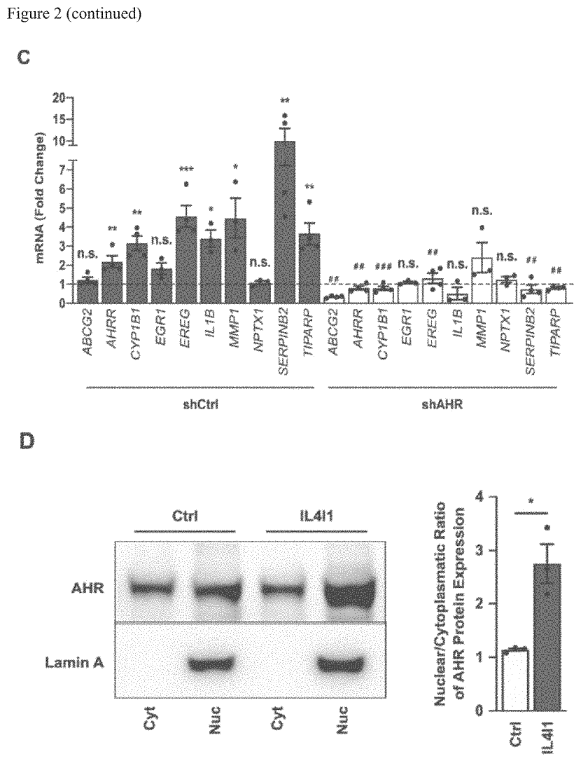

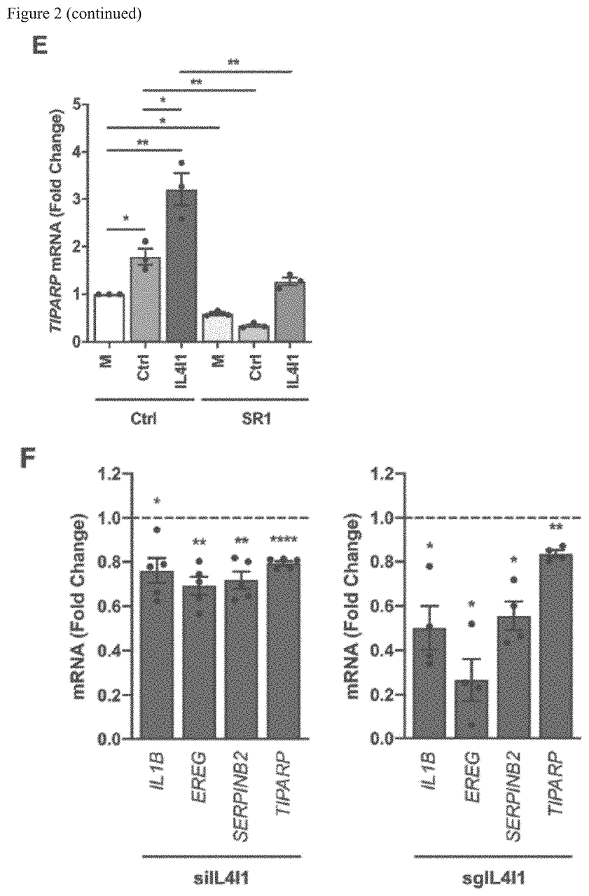

IL4I1 activates the AHR. The inventors next tested experimentally whether IL4I1 activates the AHR. For this purpose, the inventors opted for human cell cultures and tissues, as mice express an additional L-amino acid oxidase (Lao1) (Hughes, 2010) that can compensate for Il4i1 deficiency. IL4I1 protein was expressed in human GBM tissue (FIG. 2A), suggesting GBM cells as a suitable model to investigate IL4I1. The inventors compared AHR-proficient (U87-MG) with AHR-deficient (U-251MG) GBM cell lines. IL4I1 was ectopically expressed in cell lines with low endogenous IL4I1, or downregulated by RNA interference or CRISPR/Cas9 in a cell line with high endogenous IL4I1. Ectopic expression of IL4I1 enhanced its enzymatic activity, as assessed by H₂O₂ levels. In the AHR-proficient cells, IL4I1 induced the pan-tissue AHR signature (FIG. 2B, top), which was reversed by AHR knockdown (FIG. 2B, bottom). Selected AHR target genes were confirmed by qRT-PCR (FIG. 2C). To test whether IL4I1-derived metabolites activate the AHR, the inventors treated GBM cells with supernatants of cells with ectopic IL4I1. In support of the inventors' hypothesis, the supernatants elicited increased nuclear AHR localization (FIG. 2D) and target gene transcription (FIG. 2E). Conversely, IL4I1 downregulation decreased expression of the AHR target genes IL1B, EREG, SERPINB2 and TIP ARP (FIG. 2F).

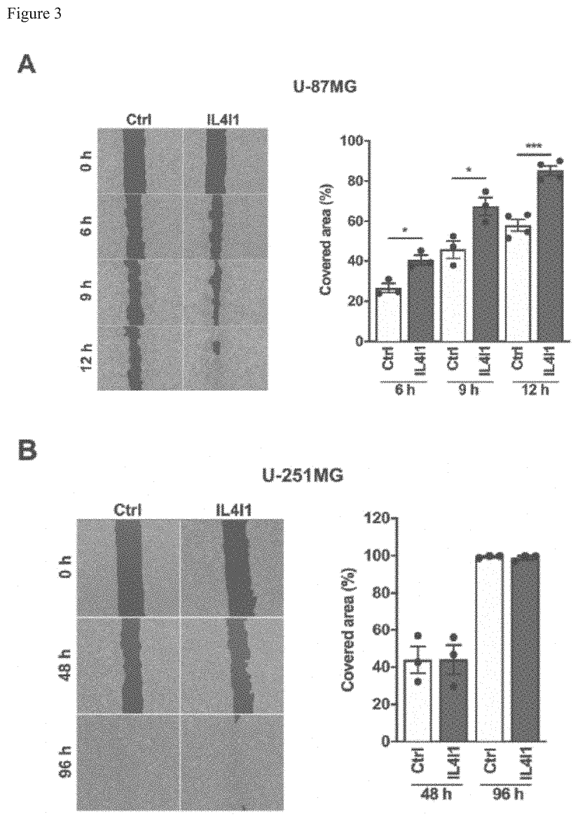

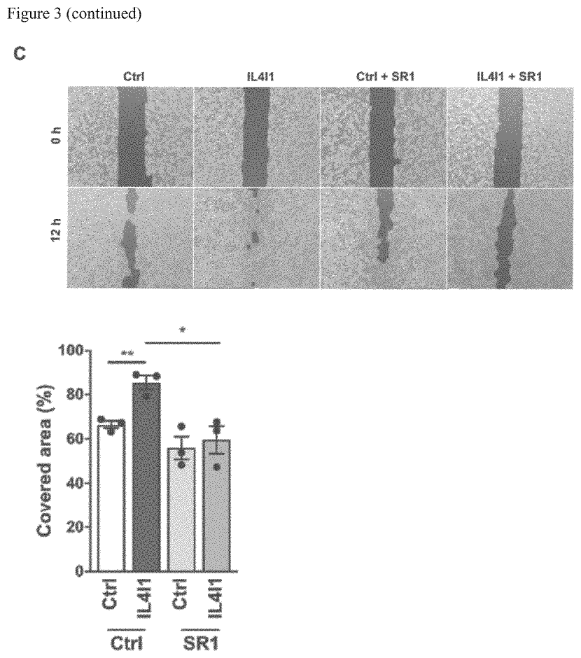

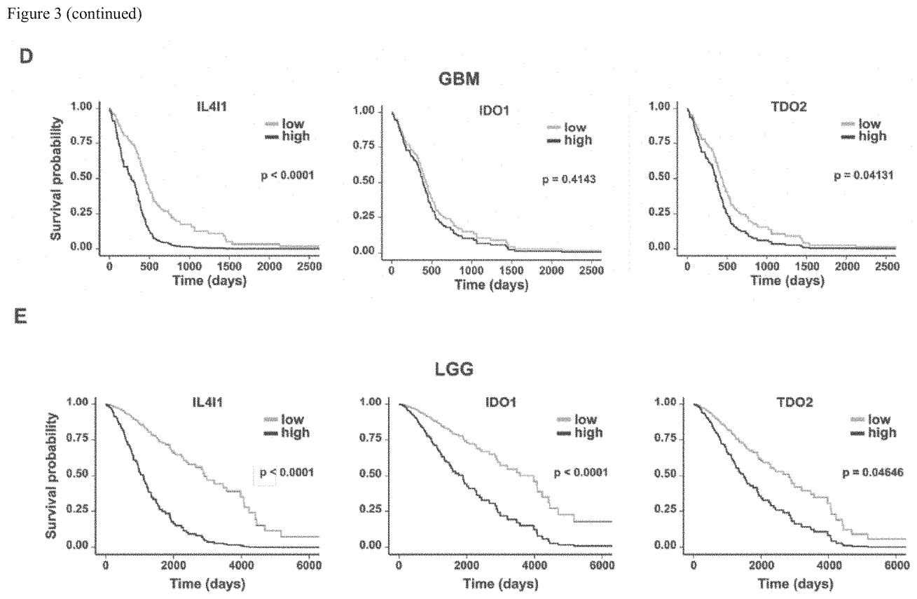

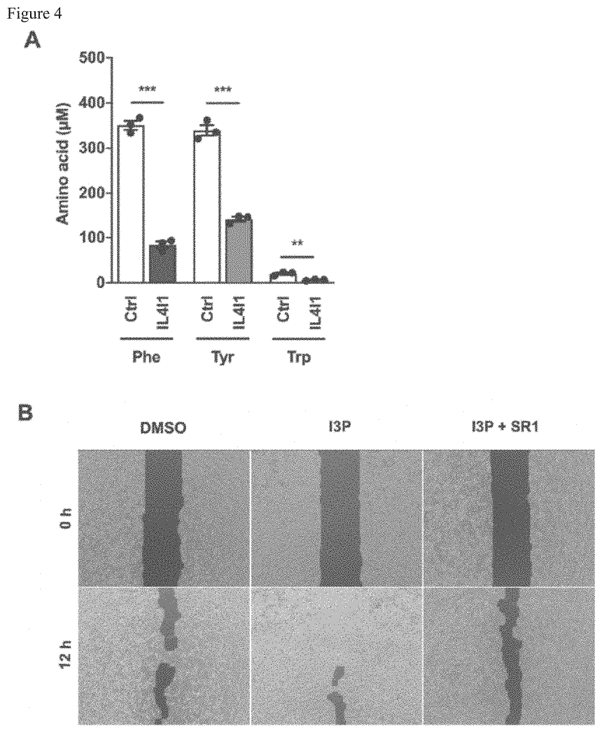

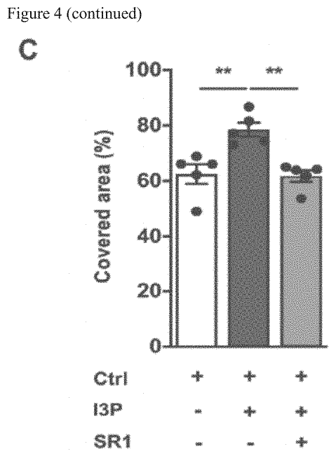

IL4I1 promotes tumor cell motility, suppresses T cell proliferation, and is associated with reduced survival in glioma patients. To test IL4I1's contribution to the functional outcomes of the AHR, the inventors first investigated the role of IL4I1 in GBM cell motility. Ectopic IL4I1 increased the motility of AHR-proficient cells (FIG. 3A), but not of AHR-deficient cells (FIG. 3B). In keeping with this result, the IL4I1-mediated motility of AHR-proficient cells was reversed by the AHR inhibitor SR1 (FIG. 3C). This experiment confirms the inventors' hypothesis that IL4I1 is a key driver of cancer cell motility and elicits major effects in immunity and cancer by signaling through the AHR axis. Through this axis, IL4I1 is linked to tumor intrinsic malignant properties, including cell motility (cancer cell migration and metastasis).

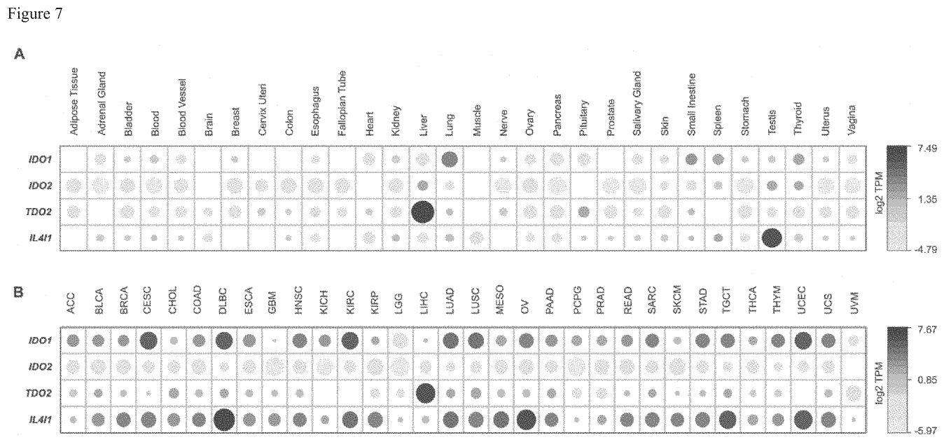

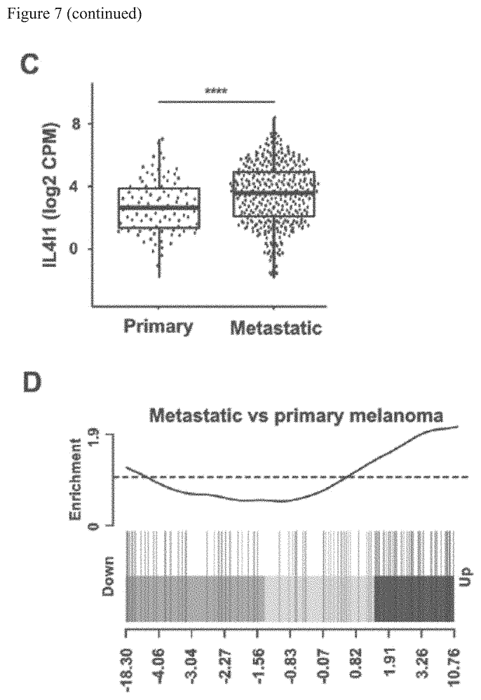

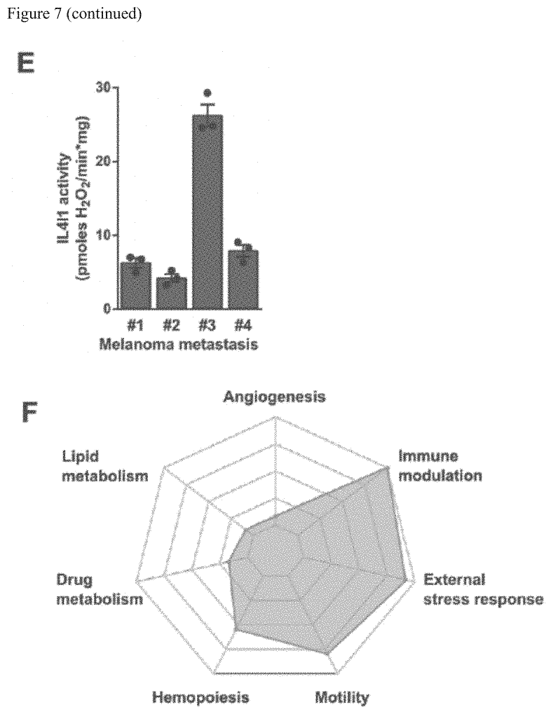

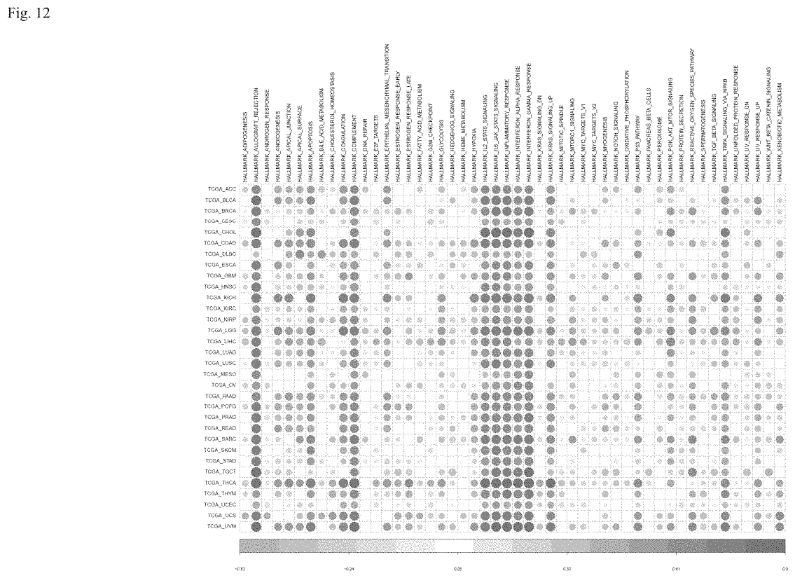

These results are further confirmed by the data shown in FIG. 12, where biological process activity scores of the Hallmark gene-sets of cancer were generated for each single patient across all 32 TCGA tumors, and were further correlated with IL4I1 expression levels. Indeed, IL4I1 is correlating highly with the enrichment scores generated for the Hallmarks of cancer gene-sets specific to cell motility. By analyzing the AAMs that comprise IL4I1, these AAMs were enriched with gene ontologies of cell motility, across all cancer types (FIG. 7F). This provides further evidence that the effect of IL4I1 on cell motility can be extrapolated across any cancer type (FIG. 7F and FIG. 12).

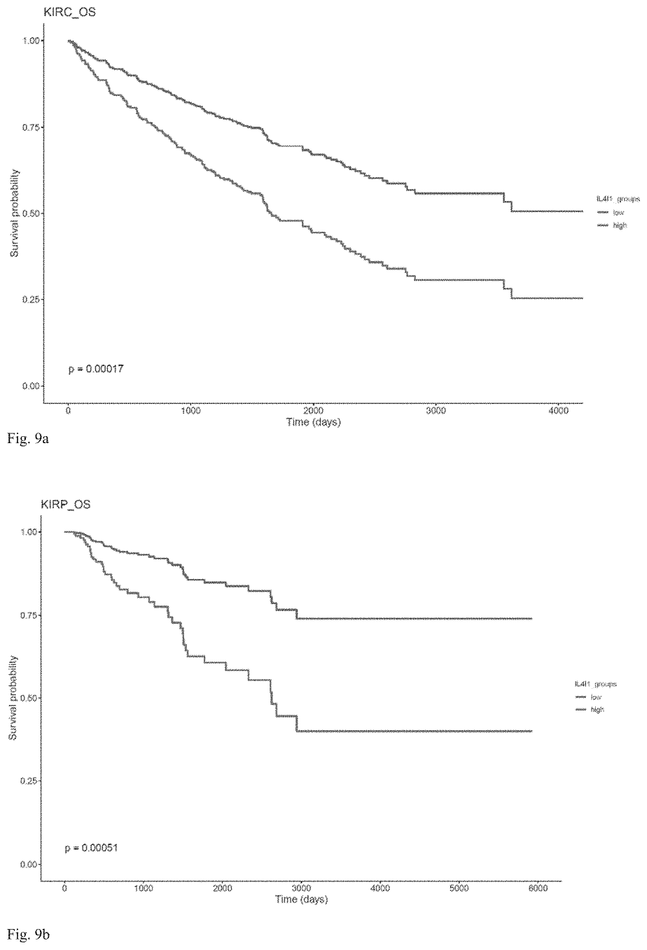

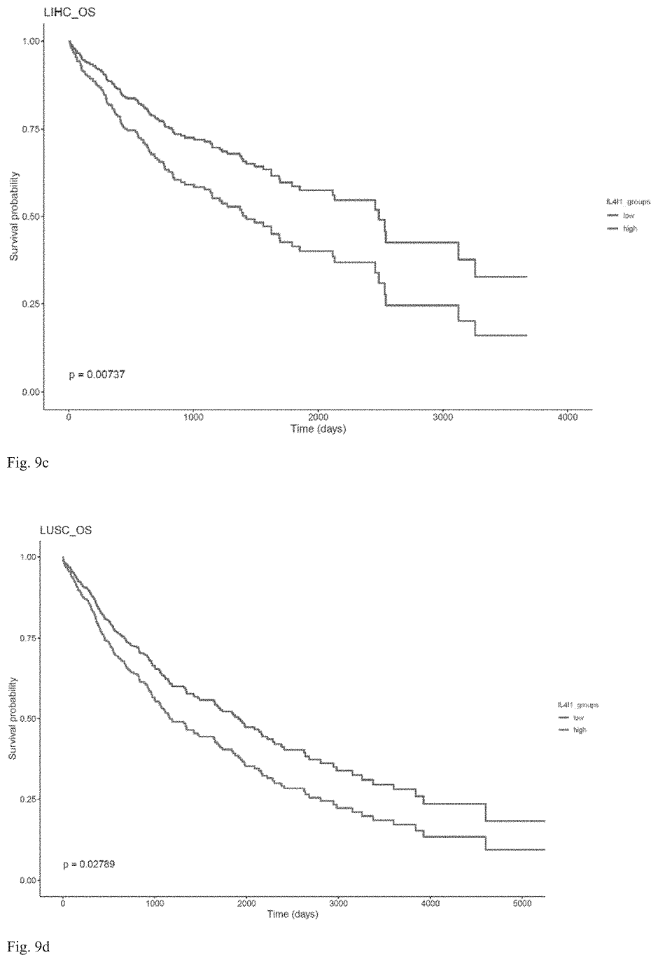

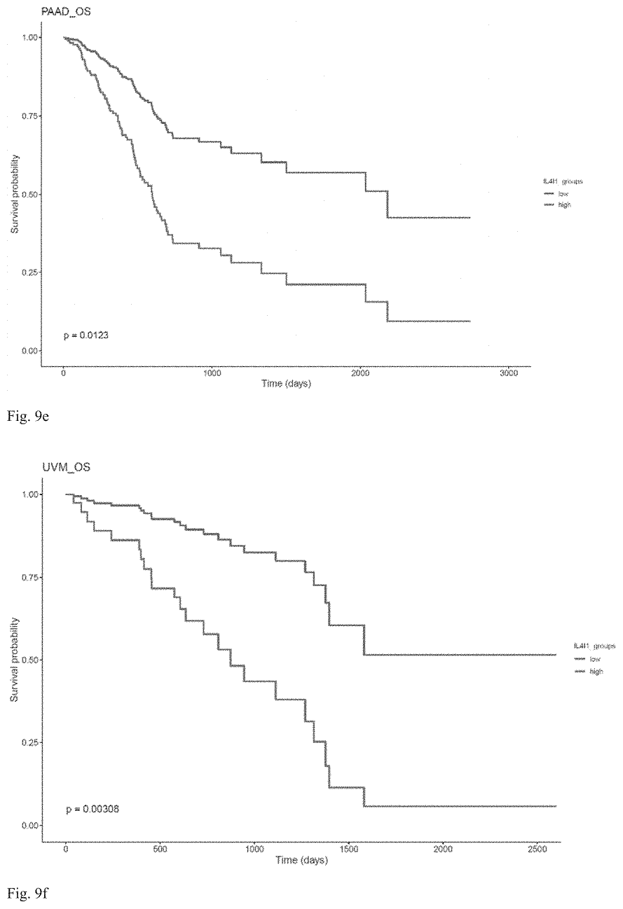

To translate the inventors' findings to patients, the inventors analyzed the association of the three AHR-activating enzymes IL4I1, IDO1, and TDO2, with overall survival probability in GBM. High IL4I1 levels associated with reduced overall survival (FIG. 3D). In fact, GBM patients with high tumoral IL4I1 expression (low survival group) had a 2.3 times higher risk of death compared to patients with low IL4I1 expression (high survival group) (FIG. 3D, and Table 2). High TDO2 levels associated with a 1.5 times higher risk of death, while no significant survival difference was associated with IDO1 transcript levels (FIG. 3E and Table S6). Similarly, in low grade glioma (LGG) high expression of IL4I1 (low survival group), IDO1 and TDO2 were all associated with a higher risk of death (FIG. 3E). The risk increase was highest for IL4I1 expression (3.3 fold), followed by IDO1 (2.4 fold), and TDO2 (1.6 fold) (FIG. 3E, and Table 2). Similarly, patients with high expression levels of IL4I1, in additional TCGA tumors show significant worse overall survival outcome compared to low IL4I1 expression (FIG. 9).

IL4I1 expression is increased in cancer and metastasis. To explore IL4I1's potential as a therapeutic target in malignancies the inventors investigated its expression in normal tissues versus primary tumors and metastases. Across most tumor entities, IL4I1 expression was enhanced in primary cancer tissues compared to normal tissues (FIGS. 7A and 7B). In the majority of tumors, IL4I1 expression was higher than IDO1 or TDO2, highlighting IL4I1 as a major Trp-catabolizing enzyme in cancer (FIG. 7B). In support of their malignant properties, IL4I1 levels (FIG. 7C) and AHR activity (FIG. 7D) were significantly higher in metastatic melanoma, as compared to primary melanoma. In agreement, IL4I1 enzymatic activity was detected in freshly excised metastatic melanoma tissue (FIG. 7E).

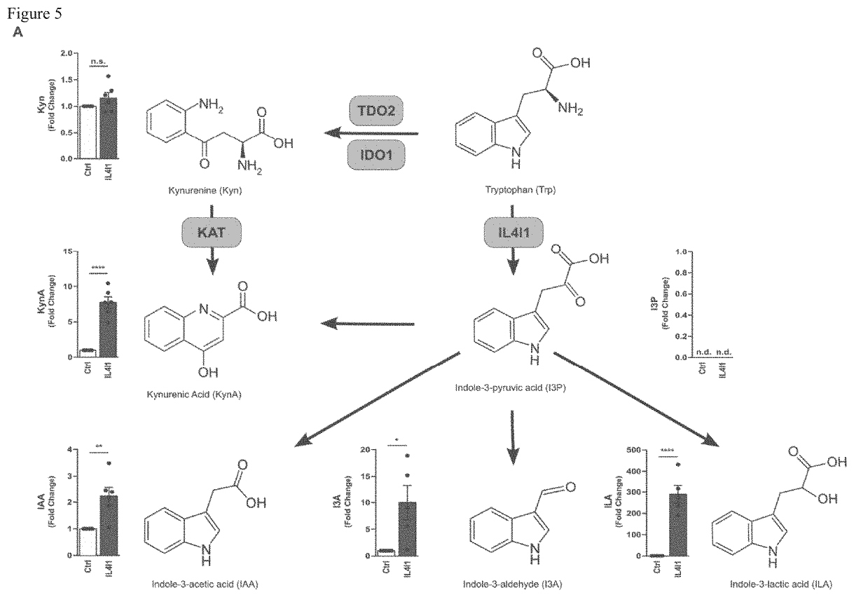

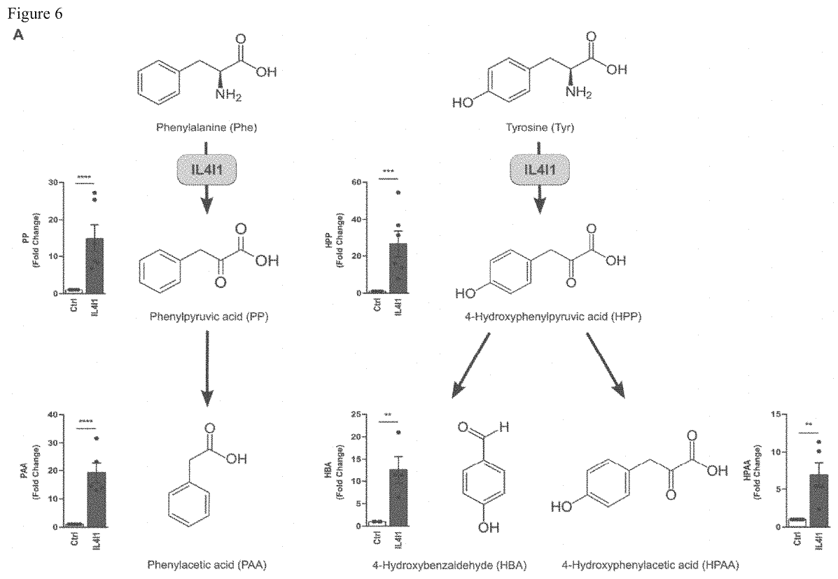

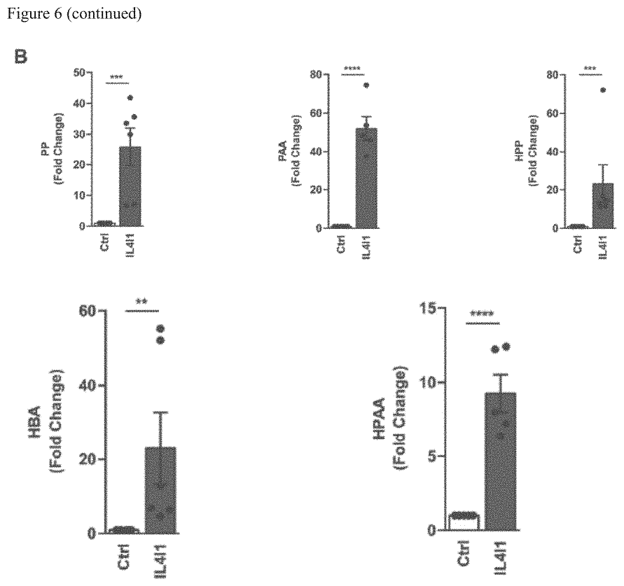

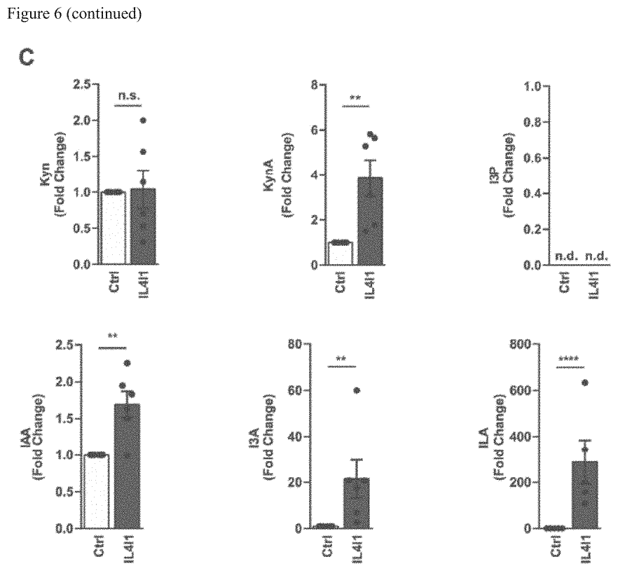

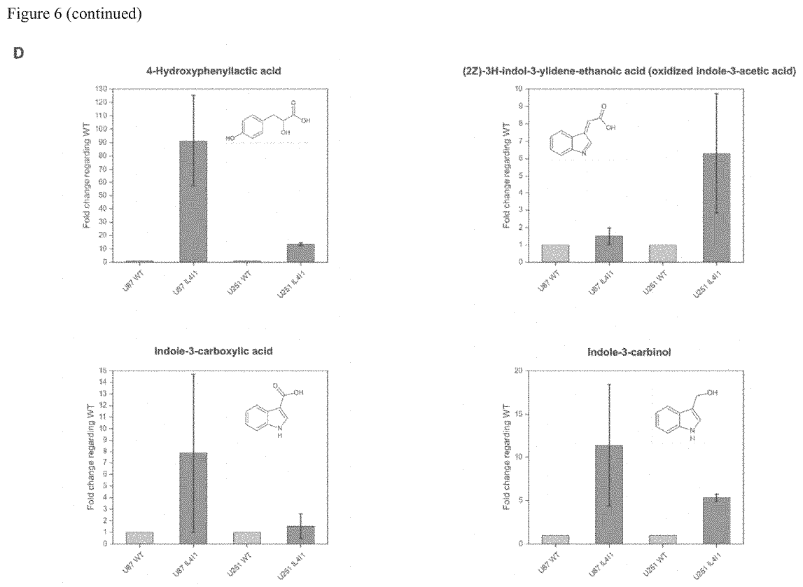

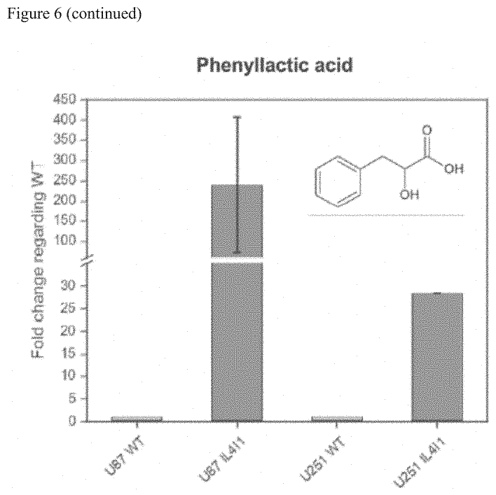

Indole-3-pyruvate gives rise to the AHR agonists kynurenic acid and indole-3-aldehyde. Surprisingly, all known IL4I1 products but I3P were detectable in supernatants of cells with ectopic IL4I1 (FIGS. 5 and 6). Yet, the inventors detected increased levels of I3P derivatives, including indole-3-acetic acid (IAA), indole-3-aldehyde (I3A) and indole-3-lactic acid (ILA) (FIGS. 5A and 6C). Moreover, IL4I1 enhanced the levels of KynA, while it did not affect Kyn levels (FIGS. 5A and 6C). KynA formation from I3P was unexpected as KynA is typically considered to originate from IDO1- or TDO2-derived Kyn (Cervenka et al., 2017; Platten et al., 2019). One reaction through which I3P could enhance KynA levels is the transamination of Kyn, as kynurenine aminotransferase (KAT) can use alpha-ketoacids as amino group acceptors (Han et al., 2004). However, Kyn concentrations were not reduced in cells with ectopic IL4I1 (FIGS. 5 and 6C), rendering this hypothesis unlikely. Interestingly, KynA formed spontaneously from I3P under cell-free conditions (data not shown). This was enhanced in the presence of H₂O₂ (data not shown), suggesting that IL4I1-derived H₂O₂ promotes I3P's conversion to the AHR agonist KynA.

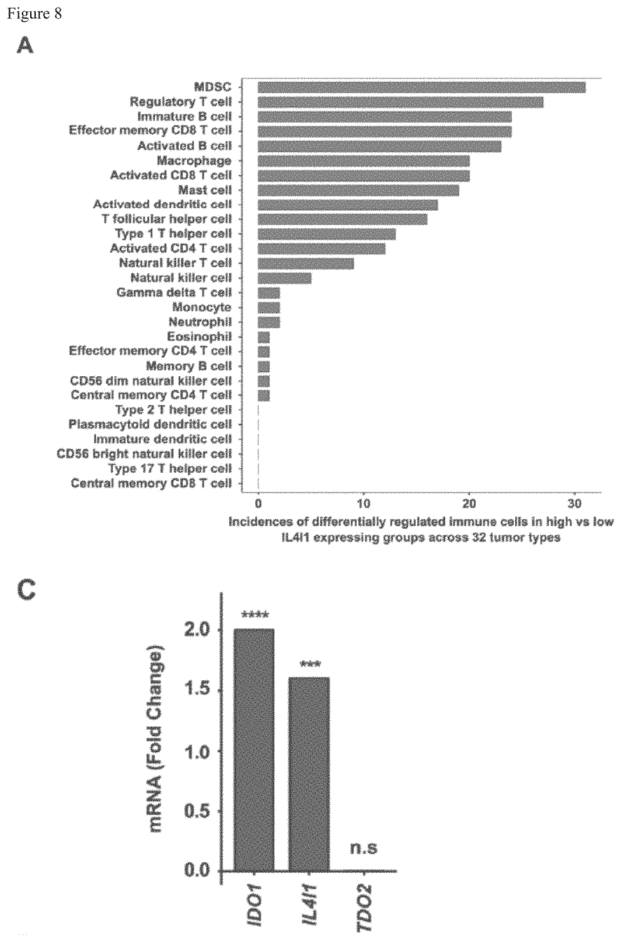

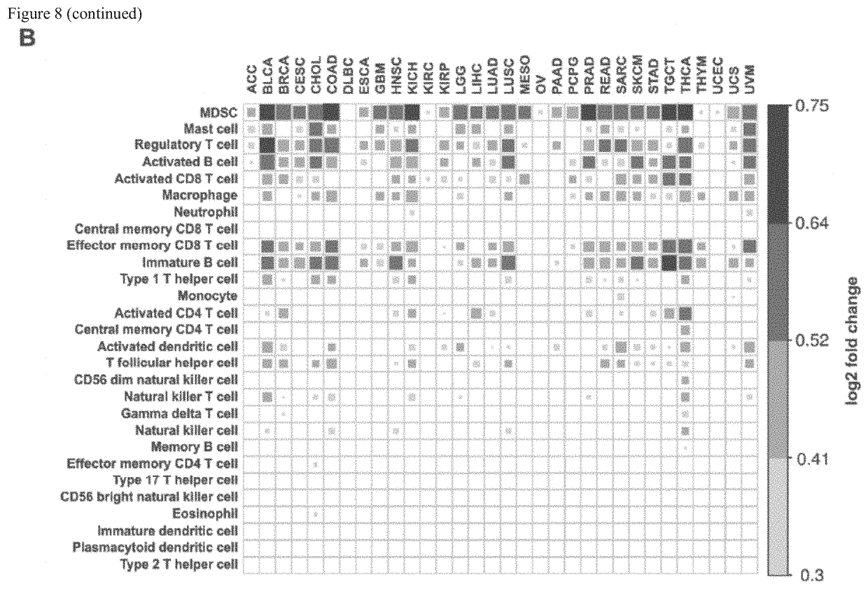

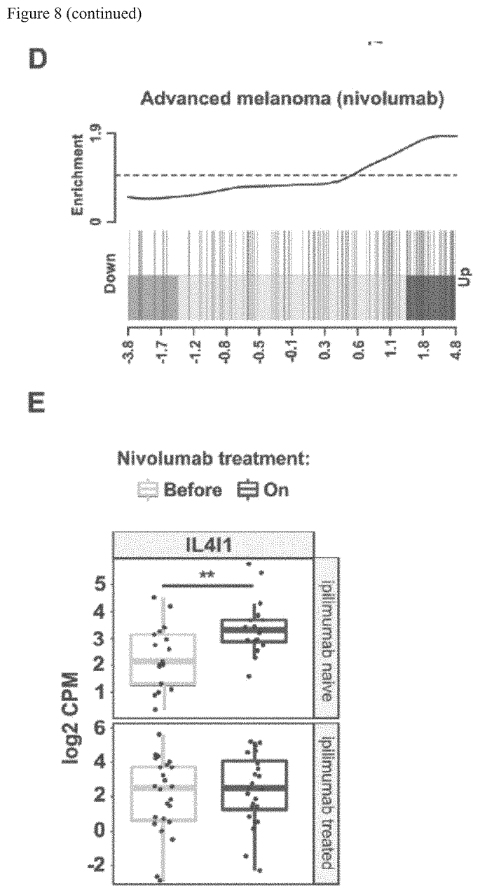

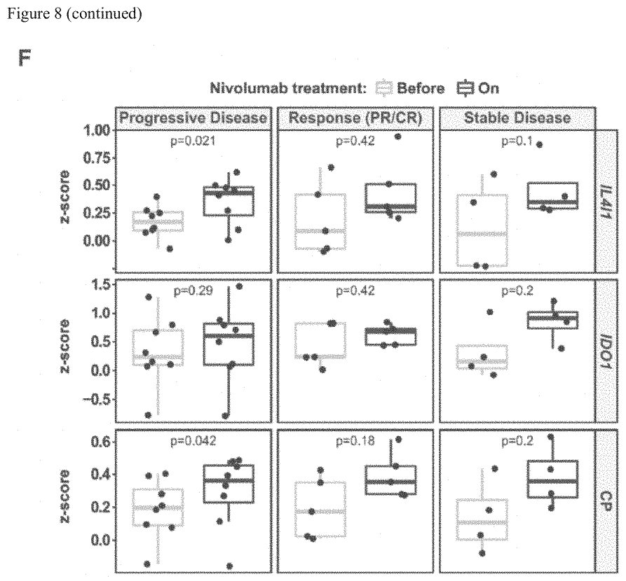

Although IL4I1 drives motility and metastasis, motility was only the third most enriched ontology group for AAMs containing IL4I1 (FIG. 7F). In fact, immune modulation was most enriched, and thus might be an even more prominent IL4I1 outcome in cancer. Across the 32 cancer types, IL4I1 high expressing tumors showed an enrichment of suppressive immune cells such as myeloid-derived suppressor cells (MDSCs) and Treg cells (FIGS. 8A and 8B).

ICB through antibodies targeting the PD-1/(programmed cell death 1 ligand 1) PD-L1 interaction has been approved for multiple cancers based on unprecedented clinical responses in a fraction of patients. However, most cancer patients do not benefit from ICB, and there is an urgent need to identify the molecular basis for ICB resistance. IL4I1's association with suppressive immune cells suggests that IL4I1 could circumvent ICB by activating an alternative immunosuppressive mechanism. Therefore, the inventors investigated whether Trp-degrading enzymes are upregulated upon ICB. The inventors found that the anti-PD-1 monoclonal antibody (mAb) nivolumab induced IL4I1 and IDO1 expression in advanced melanoma (FIG. 8C) (Riaz et al., 2017). Moreover, nivolumab treatment resulted in AHR activation (FIG. 8D), suggesting that IL4I1 signaling to the AHR represents a novel metabolic immune checkpoint that mediates ICB resistance. To further investigate this possibility, the inventors analyzed complete response (CR) information of matched before and on treatment RNA-seq data from 41 patients. Out of these, 24 patients had received the anti-CTLA4 mAb ipilimumab, prior to nivolumab initiation (Riaz et al., 2017). In agreement with the entire cohort (FIG. 8C), the inventors observed a significant IL4I1 increase in response to nivolumab in the ipilimumab-naïve patients (FIG. 8E). Of note, IL4I1 (but not IDO1) and immune checkpoint (CP) molecules were significantly induced in ipilimumab-naïve patients that developed progressive disease (PD) (FIG. 8F), suggesting that IL4I1 induction represents a resistance mechanism against ICB. In addition, the data shows that patients receiving ICB and had progressive disease showed higher IL4I1 upon ICB therapy but no significant difference in IDO1 expression (FIG. 8F). These critical findings have been recently confirmed in Sadik et. al 2020 Cell (PMID: 32818467).

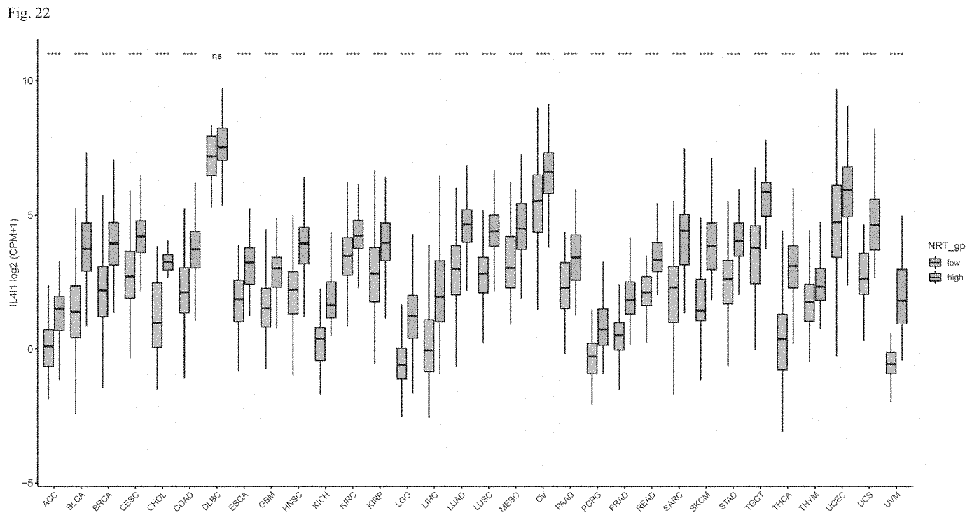

The inventors show that 11411 can be a predictor of non-response (and be used of stratifying cancer patients into non-responders and responders) to immunotherapy (FIG. 8F and FIG. 22). IL4I1 targeting constitutes a new strategy to relieve resistance to immunotherapy, in this case in the context of ICB-related cancer treatment. As mentioned above, the activity of IL4I1 can then, for example, be determined using the metabolites and tests as described above. Expression can be detected using genetic tools, such as chip analysis and primers and probes and PCR analysis, or detecting the amount of protein of IL4I1 using, for examples, antibodies, in samples, like biological fluids or tissue samples. Particularly preferred is a method according to the present invention, wherein said increase of said activity and/or expression of said IL4I1 is detected in response to an ICB immunotherapy in said patient. In the context of the present invention, it was shown for the first time that resistance to immunotherapy is actively linked to the increased activity and/or expression of IL4I1 and the IL4I1-AHR axis. The consequence of a positive finding based on this method would be the use of an IL4I1 inhibitor, or an AHR modulator for therapy.

As a further confirmation of the fact that high IL4I1 levels are associated with reduced overall survival in various types of cancers/tumors (FIGS. 3D, 3E and 9), FIGS. 8E and 8F also show that advanced melanoma patients receiving the anti-PD1 monoclonal antibody Nivolumab (PMID: 29033130), not only show higher expression of IL4I1 after receiving the Nivolumab treatment, but also higher IL4I1 expression in patients with progressive disease compared to patients with stable disease or partial and complete response (FIGS. 8E and 8F).

These results demonstrate that higher IL4I1 expression associates with worse survival outcome across different types of cancers. The examples we show underscore the effect of a high basal expression of IL4I1 on survival outcome, but also demonstrate that high IL4I1 expression induced in patients upon immunotherapeutic intervention, mediates worse clinical outcomes.

In addition, the findings that immunotherapy such as ICB induces IL4I1 are not limited to melanomas.

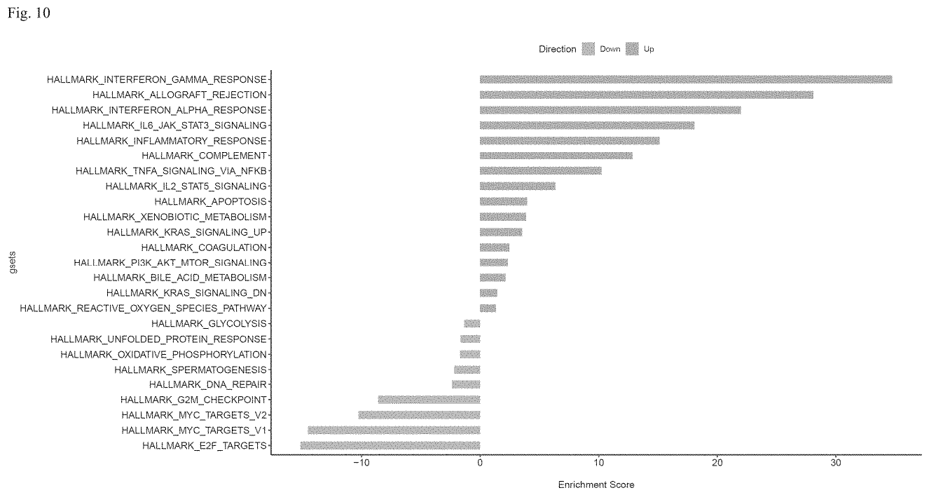

Indeed, when comparing the enrichment of the hallmark of cancer gene sets (PMID: 21546393) in the advanced melanoma patients (PMID: 29033130) before and on Nivolumab therapy, a strong upregulation of inflammatory hallmark gene signatures is observed. The strongest enrichment was for the hallmark gene set of interferon gamma response (HALLMARK_INTERFERON_GAMMA_RESPONSE) (see FIG. 10).

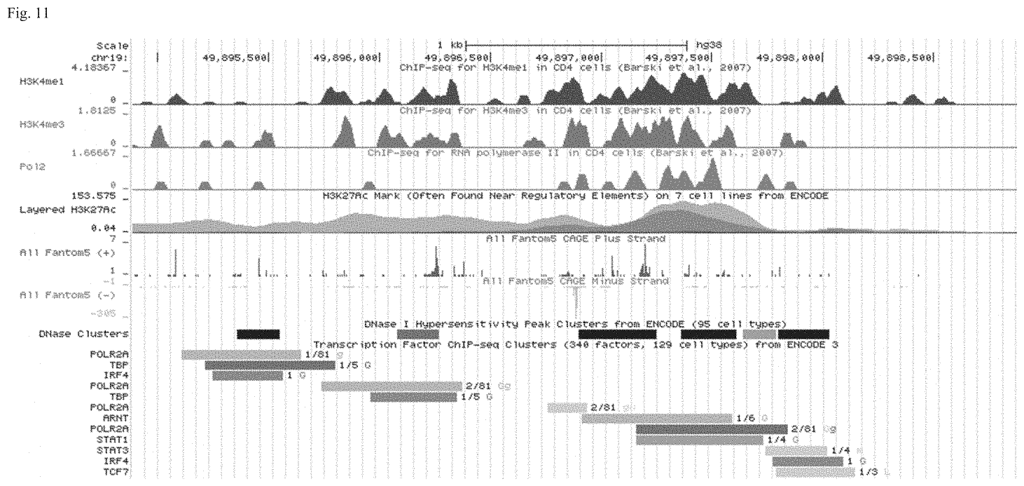

Analyzing the promoter region at the transcription start site of IL4I1, showed that the transcription factors (TFs) IRF4, STAT1 and STAT3 have promoter-binding sites in the regions with high chromatin accessibility (FIG. 11). Taken together, the strong enrichment of interferon gamma response upon immune therapy and the presence of TF binding sites of IRF4, STAT1 and STAT3 strengthen the induction of IL4I1 in response to immune therapy. Moreover, we generated single sample biological activity scores (BPA) (PMID: 31653878; PMID: 27322546) of all Hallmark gene sets across all TCGA tumors and correlated the BPA scores with the expression of IL4I1. Of note, the highest correlations across all TCGA tumors were for the immune hallmark gene sets (FIG. 12). This supports the extrapolation of the effect of immune therapeutic interventions on inducing IL4I1 expression to any TCGA tumors.

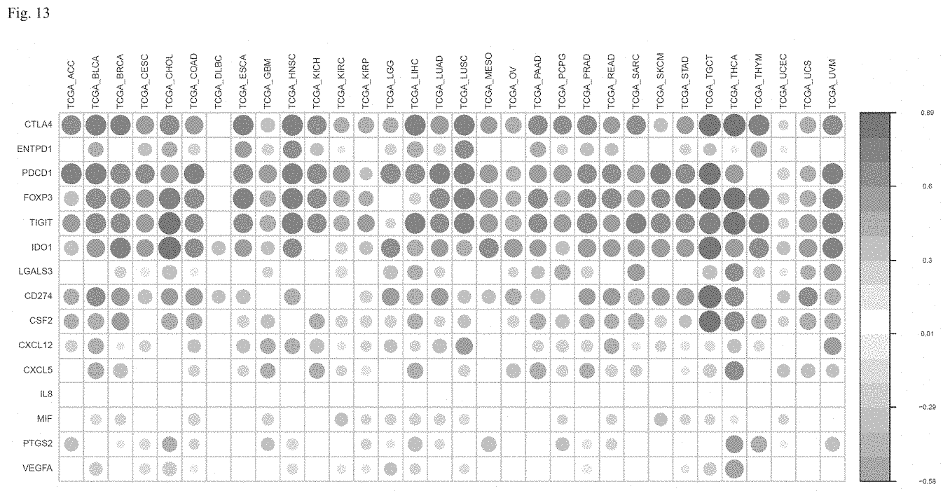

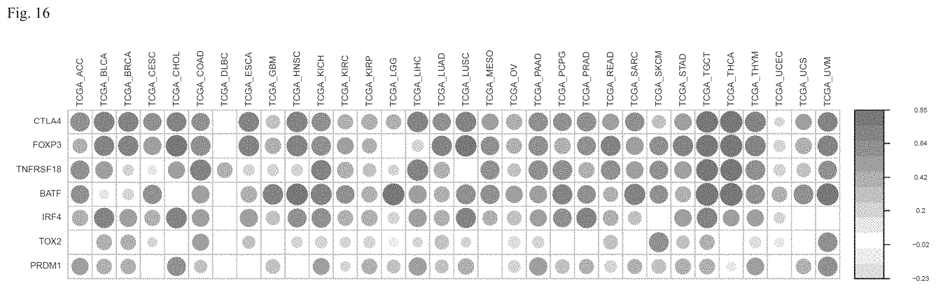

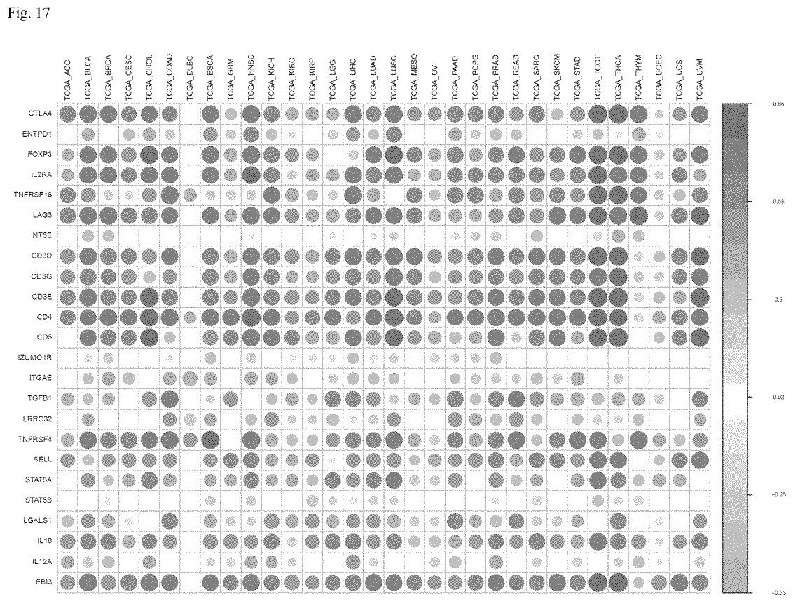

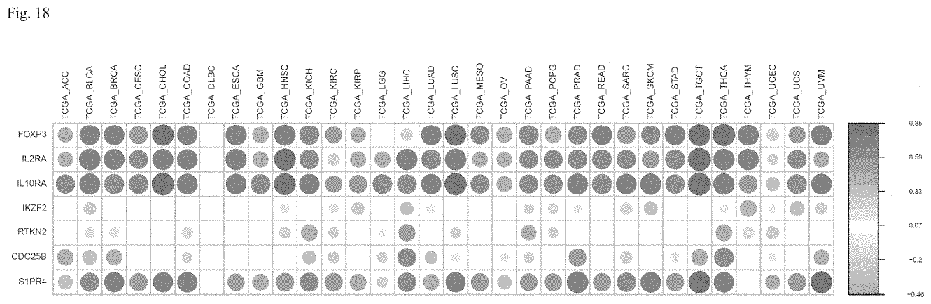

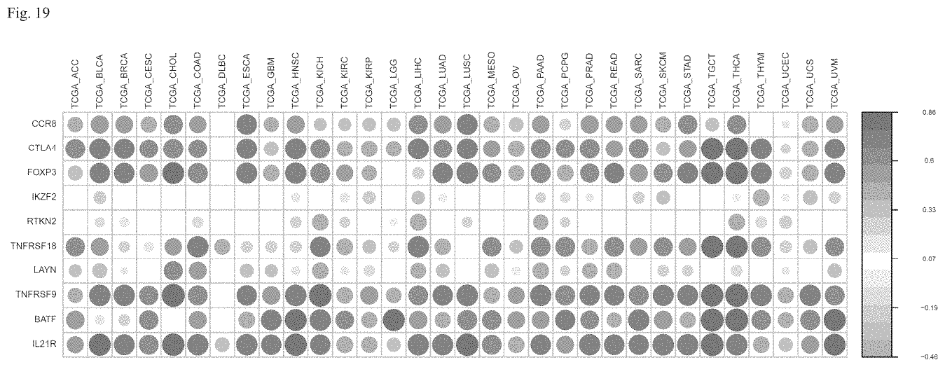

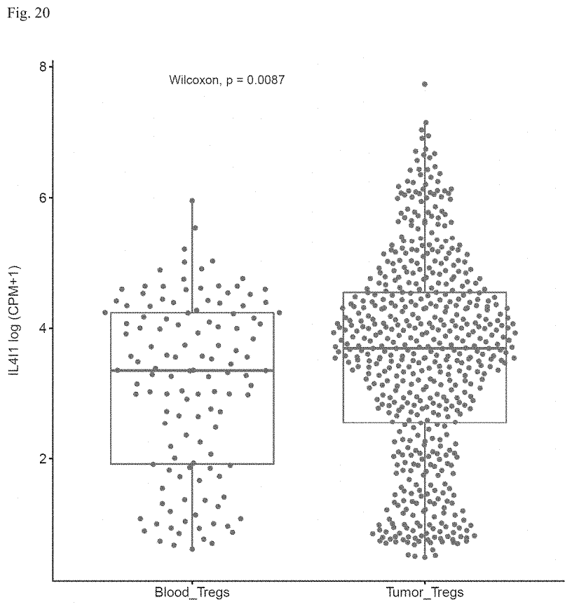

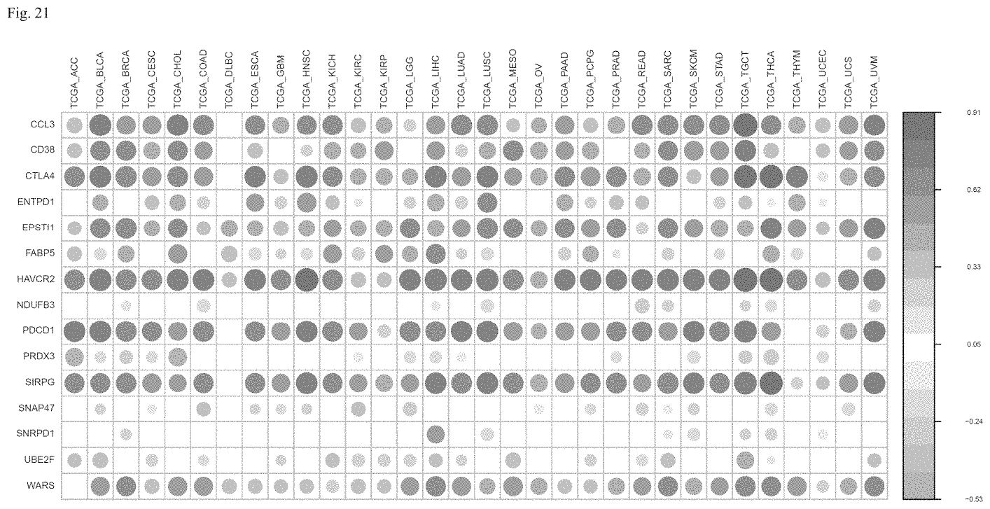

In order to further confirm that the immune suppressive effects of IL4I1 extend across all tumors, markers of immune suppression, T cell exhaustion and regulatory T cells were extracted from single cell data of infiltrating immune cells from breast cancer (PMID: 29961579; PMID: 27851913), lung tissue (PMID: 31624246) and colorectal cancer (PMID: 31341169) and blood of the colorectal cancer patients (PMID: 31341169). These markers were detected in the bulk RNA-seq data of the TCGA tumors. IL4I1 expression highly correlated with these markers across all TCGA tumors, especially FOXP3, TIGIT, LAGS, TOX2, LAYN, BATF, CTLA4, PD1 and PD-L1 (FIGS. 13-19). Of note, IL4I1's immune suppressive properties are pronounced in tumor tissue as illustrated by higher IL4I1 expression in Tregs infiltrating colorectal cancer tissue compared to Tregs circulating in the blood of the same patients (FIG. 20). Taken together, these findings show clear evidence that IL4I1-mediated immune suppression is a general phenomenon across diverse cancer tissues. Importantly, markers of immunotherapy non-response defined from single cell populations of advanced melanoma patients receiving different immune checkpoint blockers (anti-PD1, anti-CTLA-4, or a combination of both) (PMID: 30388456) correlated strongly with the IL4I1 expression in all TCGA tumors (FIG. 21). Most importantly, high expression of immunotherapy non-response signature genes associated with high IL4I1 expression (FIG. 22).

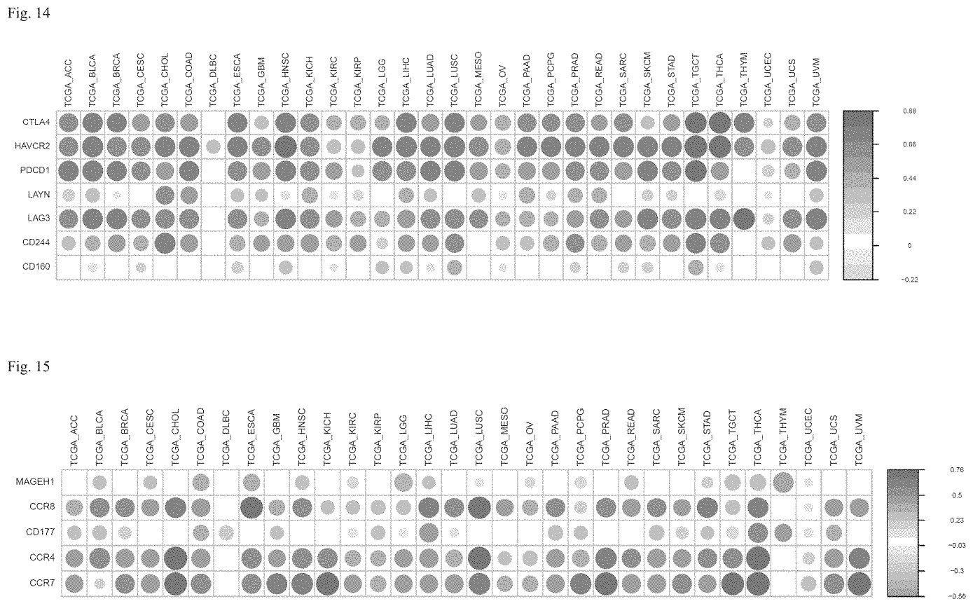

Taken together our data reveal that IL4I1 can be induced by immunotherapy in diverse tumor entities (FIG. 12). The strong association of IL4I1 with myeloid derived suppressor cells (FIGS. 8A and 8B), Tregs (FIGS. 8A and 8B) and Treg markers (FIGS. 13, 15-21) as well as T cell exhaustion markers (FIG. 14) across the TCGA tumors underscores the immunosuppressive effects of IL4I1, which is linked to resistance against immunotherapy as demonstrated by its increased expression in tumors with high immunotherapy non-response marker expression (FIG. 22).

Based on the notion that IDO1 might constitute a potential escape mechanism from anti-PD-1 ICB, a recent phase III clinical trial in advanced melanoma tested the clinical benefit of the IDO1 inhibitor epacadostat in combination with the anti-PD-1 mAb pembrolizumab (Long et al., 2019). However, this trial failed (Garber, 2018; Long et al., 2019; Muller et al., 2019; Platten et al., 2019). The major findings of the inventors are that 1) ICB induces IL4I1, which mediates therapy escape and 2) IL4I1 is a more potent AHR activator than IDO1, which may very likely explain the failure of clinical studies combining immune checkpoint blockade (ICB) with IDO1 inhibitors

Indeed, the inventors hypothesized that in light of their downstream convergence on the AHR, IL4I1 could constitute a resistance mechanism against IDO1 inhibition. The inventors propose that by activating the AHR, IL4I1 represents a resistance mechanism against ICB and/or IDO1 inhibitors. IL4I1 therefore is a new promising target for tumor therapy.

These findings allow to advantageously stratify cancer patients into groups in order to improve diagnoses and/or treatment of cancer. In particular, one aspect of the invention is a method of stratifying cancer patients into a high survival group and a low survival group comprising detecting the activity and/or expression of IL4I1 in a sample obtained from said cancer patients, wherein if said expression or enzymatic activity of said IL4I1 is increased in said patient, wherein said expression or enzymatic activity of said IL4I1 modulates the AHR, when compared to control genes e.g., one or more housekeeping genes in said sample, or a control sample, e.g. a sample derived from a healthy subject, a group of healthy subjects, a prior sample from the same patient, a different patient or patient group, the patient is in the low survival group and wherein if said expression or enzymatic activity of said IL4I1 is unchanged or decreased in said patient when compared to control genes e.g., one or more housekeeping genes in said sample, or a control sample, e.g. a sample derived from a healthy subject, a group of healthy subjects, a prior sample from the same patient, a different patient or patient group, the patient is in the high survival group. In a preferred embodiment, the increase of the activity and/or expression of said IL4I1 is detected in response to an immunotherapy in said patient. In a more preferred embodiment, the patients in the high survival group are immunotherapy responders and patients in the low survival group are immunotherapy non-responders, in particular immune checkpoint blockade (ICB) non responders.

### REFERENCES AS CITED

Agudelo, L. Z., et al. (2018). Kynurenic Acid and Gpr35 Regulate Adipose Tissue Energy Homeostasis and Inflammation. Cell metabolism 27, 378-392e375.
Aoki, R., et al. (2018). Indole-3-Pyruvic Acid, an Aryl Hydrocarbon Receptor Activator, Suppresses Experimental Colitis in Mice. J Immunol 201, 3683-3693.
Argelaguet, R., et al. (2018). Multi-Omics Factor Analysis-a framework for unsupervised integration of multi-omics data sets. Mol Syst Biol 14, e8124.
Bittinger, M. A., Nguyen, L. P., and Bradfield, C. A. (2003). Aspartate aminotransferase generates proagonists of the aryl hydrocarbon receptor. Mol Pharmacol 64, 550-556.
Boitano, A. E., et al. (2010). Aryl hydrocarbon receptor antagonists promote the expansion of human hematopoietic stem cells. Science 329, 1345-1348.
Boulland, M. L., M et al. (2007). Human IL4I1 is a secreted L-phenylalanine oxidase expressed by mature dendritic cells that inhibits T-lymphocyte proliferation. Blood 110, 220-227.
Boyle, E. I., Weng, S., Gollub, J., Jin, H., Botstein, D., Cherry, J. M., and Sherlock, G. (2004). GO::TermFinder—open source software for accessing Gene Ontology information and finding significantly enriched Gene Ontology terms associated with a list of genes. Bioinformatics 20, 3710-3715.
Brennan, J. C., et al. (2015). Development of Species-Specific Ah Receptor-Responsive Third Generation CALUX Cell Lines with Enhanced Responsiveness and Improved Detection Limits. Environ Sci Technol 49, 11903-11912.
Bui, Q. C., and Sloot, P. M. (2012). A robust approach to extract biomedical events from literature. Bioinformatics 28, 2654-2661.
Carbonnelle-Puscian, A., et al. (2009). The novel immunosuppressive enzyme IL4I1 is expressed by neoplastic cells of several B-cell lymphomas and by tumor-associated macrophages. Leukemia 23, 952-960.
Carvalho, B., Bengtsson, H., Speed, T. P., and Irizarry, R. A. (2007). Exploration, normalization, and genotype calls of high-density oligonucleotide SNP array data. Biostatistics 8, 485-499.
Cervantes-Barragan, L., et al. (2017). Lactobacillus reuteri induces gut intraepithelial CD4(+)CD8alphaalpha(+) T cells. Science 357, 806-810.
Cervenka, I., Agudelo, L. Z., and Ruas, J. L. (2017). Kynurenines: Tryptophan's metabolites in exercise, inflammation, and mental health. Science 357.
Charoentong, P., Finotello, F., Angelova, M., Mayer, C., Efremova, M., Rieder, D., Hackl, H., and Trajanoski, Z. (2017). Pan-cancer Immunogenomic Analyses Reveal Genotype-Immunophenotype Relationships and Predictors of Response to Checkpoint Blockade. Cell Rep 18, 248-262.
Chen, J. Y., Li, C. F., Kuo, C. C., Tsai, K. K., Hou, M. F., and Hung, W. C. (2014). Cancer/stroma interplay via cyclooxygenase-2 and indoleamine 2,3-dioxygenase promotes breast cancer progression. Breast cancer research: BCR 16, 410.
Colaprico, A., et al. (2016). TCGAbiolinks: an R/Bioconductor package for integrative analysis of TCGA data. Nucleic Acids Res 44, e71.
D'Amato, N.C., et al. (2015). A TDO2-AhR signaling axis facilitates anoikis resistance and metastasis in triple-negative breast cancer. Cancer Res 75, 4651-4664.
Davis, S., and Meltzer, P. S. (2007). GEOquery: a bridge between the Gene Expression Omnibus (GEO) and BioConductor. Bioinformatics 23, 1846-1847.
Denison, M. S., and Nagy, S. R. (2003). Activation of the aryl hydrocarbon receptor by structurally diverse exogenous and endogenous chemicals. Annu Rev Pharmacol Toxicol 43, 309-334.
DiNatale, B. C., et al. (2010). Kynurenic acid is a potent endogenous aryl hydrocarbon receptor ligand that synergistically induces interleukin-6 in the presence of inflammatory signaling. Toxicol Sci 115, 89-97.
Dodd, D., et al. (2017). A gut bacterial pathway metabolizes aromatic amino acids into nine circulating metabolites. Nature 551, 648-652.
Du, P., Kibbe, W. A., and Lin, S. M. (2008). lumi: a pipeline for processing Illumina microarray. Bioinformatics 24, 1547-1548.
Edgar, R., Domrachev, M., and Lash, A. E. (2002). Gene Expression Omnibus: NCBI gene expression and hybridization array data repository. Nucleic Acids Res 30, 207-210.
Esser, C., Rannug, A., and Stockinger, B. (2009). The aryl hydrocarbon receptor in immunity. Trends Immunol 30, 447-454.
Fernandez-Salguero, P., et al. (1995). Immune system impairment and hepatic fibrosis in mice lacking the dioxin-binding Ah receptor. Science 268, 722-726.
Fong, M. Y., McDunn, J., and Kakar, S. S. (2011). Identification of metabolites in the normal ovary and their transformation in primary and metastatic ovarian cancer. PLoS ONE 6, 1-12.
Gabriely, G., Wheeler, M. A., Takenaka, M. C., and Quintana, F. J. (2017). Role of AHR and HIF-1alpha in Glioblastoma Metabolism. Trends Endocrinol Metab 28, 428-436.
Gagliani, N., et al. (2015). Th17 cells transdifferentiate into regulatory T cells during resolution of inflammation. Nature 523, 221-225.
Garber, K. (2018). A new cancer immunotherapy suffers a setback. Science 360, 588.
Gautier, L., Cope, L., Bolstad, B. M., and Irizarry, R. A. (2004). affy—analysis of Affymetrix GeneChip data at the probe level. Bioinformatics 20, 307-315.
Geback, T., Schulz, M. M., Koumoutsakos, P., and Detmar, M. (2009). TScratch: a novel and simple software tool for automated analysis of monolayer wound healing assays. Biotechniques 46, 265-274.
Goeman, J. J., and Finos, L. (2012). The inheritance procedure: multiple testing of tree-structured hypotheses. Stat Appl Genet Mol Biol 11, Article 11.
Goeman, J. J., van de Geer, S. A., de Kort, F., and van Houwelingen, H. C. (2004). A global test for groups of genes: testing association with a clinical outcome. Bioinformatics 20, 93-99.
Greene, L. I., Bruno, T. C., Christenson, J. L., D'Alessandro, A., Culp-Hill, R., Torkko, K., Borges, V. F., Slansky, J. E., and Richer, J. K. (2019). A role for tryptophan-2,3-dioxygenase in CD8 T-cell suppression and evidence of tryptophan catabolism in breast cancer patient plasma. Molecular Cancer Research 17, 131-139.
Gu, Z., Eils, R., and Schlesner, M. (2016). Complex heatmaps reveal patterns and correlations in multidimensional genomic data. Bioinformatics 32, 2847-2849.
Gu, Z., Gu, L., Eils, R., Schlesner, M., and Brors, B. (2014). circlize Implements and enhances circular visualization in R. Bioinformatics 30, 2811-2812.
Gutierrez-Vazquez, C., and Quintana, F. J. (2018). Regulation of the Immune Response by the Aryl Hydrocarbon Receptor. Immunity 48, 19-33.
Han, Q., Li, J., and Li, J. (2004). pH dependence, substrate specificity and inhibition of human kynurenine aminotransferase I. Eur J Biochem 271, 4804-4814.
Hanzelmann, S., Castelo, R., and Guinney, J. (2013). GSVA: gene set variation analysis for microarray and RNA-seq data. BMC bioinformatics 14, 7.
Hothorn, T., and Zeileis, A. (2008). Generalized maximally selected statistics. Biometrics 64, 1263-1269.
Huang, J., et al. (2016). Serum metabolomic profiling of prostate cancer risk in the prostate, lung, colorectal, and ovarian cancer screening trial. British journal of cancer 115, 1087-1095.
Hubbard, T. D., Murray, I. A., and Perdew, G. H. (2015). Indole and Tryptophan Metabolism: Endogenous and Dietary Routes to Ah Receptor Activation. Drug Metab Dispos 43, 1522-1535.
Hughes, A. L. (2010). Origin and diversification of the L-amino oxidase family in innate immune defenses of animals. Immunogenetics 62, 753-759.
Kanehisa, M., and Goto, S. (2000). KEGG: kyoto encyclopedia of genes and genomes. Nucleic Acids Res 28, 27-30.
Kiss, E. A., Vonarbourg, C., Kopfmann, S., Hobeika, E., Finke, D., Esser, C., and Diefenbach, A. (2011). Natural aryl hydrocarbon receptor ligands control organogenesis of intestinal lymphoid follicles. Science 334, 1561-1565.
Langfelder, P., and Horvath, S. (2008). WGCNA: an R package for weighted correlation network analysis. BMC bioinformatics 9, 559.
Lasoudris, F., Cousin, C., Prevost-Blondel, A., Martin-Garcia, N., Abd-Alsamad, I., Ortonne, N., Farcet, J-P., Castellano F., Molinier-Frenkel, V. (2011). IL4I1: an inhibitor of the CD8⁺ antitumor T-cell response in vivo, Eur J Immunol. Jun;41(6):1629-38)
Law, C. W., Alhamdoosh, M., Su, S., Dong, X., Tian, L., Smyth, G. K., and Ritchie, M. E. (2016). RNA-seq analysis is easy as 1-2-3 with limma, Glimma and edgeR. F1000Res 5.
Law, C. W., Chen, Y., Shi, W., and Smyth, G. K. (2014). voom: Precision weights unlock linear model analysis tools for RNA-seq read counts. Genome biology 15, R29.
Lemieux, G. A., Cunningham, K. A., Lin, L., Mayer, F., Werb, Z., and Ashrafi, K. (2015). Kynurenic acid is a nutritional cue that enables behavioral plasticity. Cell 160, 119-131.
Lemos, H., Huang, L., Prendergast, G. C., and Mellor, A. L. (2019). Immune control by amino acid catabolism during tumorigenesis and therapy. Nature Reviews Cancer 19, 162-175.
Li, B., and Dewey, C. N. (2011). RSEM: accurate transcript quantification from RNA-Seq data with or without a reference genome. BMC bioinformatics 12, 323.
Li, Y., et al. (2011). Exogenous stimuli maintain intraepithelial lymphocytes via aryl hydrocarbon receptor activation. Cell 147, 629-640.
Lin, S. M., Du, P., Huber, W., and Kibbe, W. A. (2008). Model-based variance-stabilizing transformation for Illumina microarray data. Nucleic Acids Res 36, e11.
Liu, Y., et al. (2018). Tumor-Repopulating Cells Induce PD-1 Expression in CD8(+) T Cells by Transferring Kynurenine and AhR Activation. Cancer cell 33, 480-494.e487.
Locasale, J. W., et al. (2012). Metabolomics of human cerebrospinal fluid identifies signatures of malignant glioma. Molecular and Cellular Proteomics 11, 1-12.
Long, G. V., et al. (2019). Epacadostat plus pembrolizumab versus placebo plus pembrolizumab in patients with unresectable or metastatic melanoma (ECHO-301/KEYNOTE-252): a phase 3, randomised, double-blind study. The Lancet Oncology 20, 1083-1097.
Marshall, N. B., and Kerkvliet, N. I. (2010). Dioxin and immune regulation: emerging role of aryl hydrocarbon receptor in the generation of regulatory T cells. Annals of the New York Academy of Sciences 1183, 25-37.
Mason, J. M., Naidu, M. D., Barcia, M., Porti, D., Chavan, S. S., and Chu, C. C. (2004). IL-4-induced gene-1 is a leukocyte L-amino acid oxidase with an unusual acidic pH preference and lysosomal localization. J Immunol 173, 4561-4567.
Mayer, A. K., et al. (2019). Homozygous stop mutation in AHR causes autosomal recessive foveal hypoplasia and infantile nystagmus. Brain 142, 1528-1534.
McGettrick, A. F., et al. (2016). Trypanosoma brucei metabolite indolepyruvate decreases HIF-1α and glycolysis in macrophages as a mechanism of innate immune evasion. Proceedings of the National Academy of Sciences of the United States of America 113, E7778-E7787.
Mezrich, J. D., Fechner, J. H., Zhang, X., Johnson, B. P., Burlingham, W. J., and Bradfield, C. A. (2010). An Interaction between Kynurenine and the Aryl Hydrocarbon Receptor Can Generate Regulatory T Cells. J Immunol 185, 3190-3198.
Molinier-Frenkel, V., Prevost-Blondel, A., and Castellano, F. (2019). The IL4I1 Enzyme: A New Player in the Immunosuppressive Tumor Microenvironment. Cells 8.
Muller, A. J., Manfredi, M. G., Zakharia, Y., and Prendergast, G. C. (2019). Inhibiting IDO pathways to treat cancer: lessons from the ECHO-301 trial and beyond. Semin Immunopathol 41, 41-48.
Murray, I. A., Patterson, A. D., and Perdew, G. H. (2014). Aryl hydrocarbon receptor ligands in cancer: Friend and foe. Nature Reviews Cancer 14, 801-814.
Natividad, J. M., et al. (2018). Impaired Aryl Hydrocarbon Receptor Ligand Production by the Gut Microbiota Is a Key Factor in Metabolic Syndrome. Cell metabolism 28, 737-749 e734.
Neale, P. A., et al. (2017). Development of a bioanalytical test battery for water quality monitoring: Fingerprinting identified micropollutants and their contribution to effects in surface water. Water Res 123, 734-750.
Neamah, W. H., et al. (2019). AhR Activation Leads to Massive Mobilization of Myeloid-Derived Suppressor Cells with Immunosuppressive Activity through Regulation of CXCR2 and MicroRNA miR-150-5p and miR-543-3p That Target Anti-Inflammatory Genes. The Journal of Immunology, ji1900291.
Nguyen, L. P., and Bradfield, C. A. (2008). The search for endogenous activators of the aryl hydrocarbon receptor. Chem Res Toxicol 21, 102-116.
Novikov, O., et al. (2016). An Aryl Hydrocarbon Receptor-Mediated Amplification Loop That Enforces Cell Migration in ER-/PR-/Her2-Human Breast Cancer Cells. Mol Pharmacol 90, 674-688.
Opitz, C. A., et al. (2011). An endogenous tumour-promoting ligand of the human aryl hydrocarbon receptor. Nature 478, 197-203.
Phipson, B., Lee, S., Majewski, I. J., Alexander, W. S., and Smyth, G. K. (2016). Robust Hyperparameter Estimation Protects against Hypervariable Genes and Improves Power to Detect Differential Expression. Ann Appl Stat 10, 946-963.
Plaisier, C. L., et al. (2016). Causal Mechanistic Regulatory Network for Glioblastoma Deciphered Using Systems Genetics Network Analysis. Cell systems 3, 172-186.
Platten, M., Nollen, E. A. A., Rohrig, U. F., Fallarino, F., and Opitz, C. A. (2019). Tryptophan metabolism as a common therapeutic target in cancer, neurodegeneration and beyond. Nature reviews Drug discovery.
Politi, V., Lavaggi, M. V., Di Stazio, G., and Margonelli, A. (1991). Indole-3-pyruvic acid as a direct precursor of kynurenic acid. Adv Exp Med Biol 294, 515-518.
Psachoulia, K., Chamberlain, K. A., Heo, D., Davis, S. E., Paskus, J. D., Nanescu, S. E., Dupree, J. L., Wynn, T. A., and Huang, J. K. (2016). IL4I1 augments CNS remyelination and axonal protection by modulating T cell driven inflammation. Brain 139, 3121-3136.
Quintana, F. J., Basso, A. S., Iglesias, A. H., Korn, T., Farez, M. F., Bettelli, E., Caccamo, M., Oukka, M., and Weiner, H. L. (2008). Control of T(reg) and T(H)17 cell differentiation by the aryl hydrocarbon receptor. Nature 453, 65-71.
Quintana, F. J., Murugaiyan, G., Farez, M. F., Mitsdoerffer, M., Tukpah, A. M., Burns, E. J., and Weiner, H. L. (2010). An endogenous aryl hydrocarbon receptor ligand acts on dendritic cells and T cells to suppress experimental autoimmune encephalomyelitis. Proc Natl Acad Sci USA 107, 20768-20773.
Rentas, S., et al. (2016). Musashi-2 attenuates AHR signalling to expand human haematopoietic stem cells. Nature 532, 508-511.
Riaz, N., et al. (2017). Tumor and Microenvironment Evolution during Immunotherapy with Nivolumab. Cell 171, 934-949 e916.
Ritchie, M. E., Phipson, B., Wu, D., Hu, Y., Law, C. W., Shi, W., and Smyth, G. K. (2015). limma powers differential expression analyses for RNA-sequencing and microarray studies. Nucleic Acids Res 43, e47.
Robinson, M. D., McCarthy, D. J., and Smyth, G. K. (2010). edgeR: a Bioconductor package for differential expression analysis of digital gene expression data. Bioinformatics 26, 139-140.
Robinson, M. D., and Oshlack, A. (2010). A scaling normalization method for differential expression analysis of RNA-seq data. Genome biology 11, R25.
Rothhammer, V., et al. (2018). Microglial control of astrocytes in response to microbial metabolites. Nature 557, 724-728.
Rothhammer, V., and Quintana, F. J. (2019). The aryl hydrocarbon receptor: an environmental sensor integrating immune responses in health and disease. Nat Rev Immunol.
Russi, P., Carla, V., and Moroni, F. (1989). Indolpyruvic acid administration increases the brain content of kynurenic acid. Is this a new avenue to modulate excitatory amino acid receptors in vivo? Biochem Pharmacol 38, 2405-2409.
Schwarcz, R., Bruno, J. P., Muchowski, P. J., and Wu, H. Q. (2012). Kynurenines in the mammalian brain: when physiology meets pathology. Nature reviews Neuroscience 13, 465-477.
Smirnova, A., et al. (2016). Evidence for New Light-Independent Pathways for Generation of the Endogenous Aryl Hydrocarbon Receptor Agonist FICZ. Chemical research in toxicology 29, 75-86.
Stockinger, B., Di Meglio, P., Gialitakis, M., and Duarte, J. H. (2014). The aryl hydrocarbon receptor: multitasking in the immune system. Annu Rev Immunol 32, 403-432.
Stone, T. W., Stoy, N., and Darlington, L. G. (2013). An expanding range of targets for kynurenine metabolites of tryptophan. Trends Pharmacol Sci 34, 136-143.
Takenaka, M. C., et al. (2019). Control of tumor-associated macrophages and T cells in glioblastoma via AHR and CD39. Nature neuroscience 22, 729-740.
Wang, G. L., Jiang, B. H., Rue, E. A., and Semenza, G. L. (1995). Hypoxia-inducible factor 1 is a basic-helix-loop-helix-PAS heterodimer regulated by cellular 02 tension. Proc Natl Acad Sci USA 92, 5510-5514.
Wermter, J., Tomanek, K., and Hahn, U. (2009). High-performance gene name normalization with GeNo. Bioinformatics 25, 815-821.
Wincent, E., et al. (2012). Inhibition of cytochrome P4501-dependent clearance of the endogenous agonist FICZ as a mechanism for activation of the aryl hydrocarbon receptor. Proc Natl Acad Sci USA 109, 4479-4484.
Wu, D., et al. (2010). ROAST: rotation gene set tests for complex microarray experiments. Bioinformatics 26, 2176-2182.
Xiang, Z., et al. (2019). A positive feedback between IDO1 metabolite and COL12A1 via MAPK pathway to promote gastric cancer metastasis. J Exp Clin Cancer Res 38, 314.
Yang, X., et al. (2011). A public genome-scale lentiviral expression library of human ORFs. Nat Methods 8, 659-661.
Yang, Y., et al. (2015). Relation between chemotaxis and consumption of amino acids in bacteria. Mol Microbiol 96, 1272-1282.
Yu, G., et al. (2010). GOSemSim: an R package for measuring semantic similarity among GO terms and gene products. Bioinformatics 26, 976-978.
Yu, G., Wang, L. G., Han, Y., and He, Q. Y. (2012). clusterProfiler: an R package for comparing biological themes among gene clusters. OMICS 16, 284-287.
Zelante, T., et al. (2013). Tryptophan catabolites from microbiota engage aryl hydrocarbon receptor and balance mucosal reactivity via interleukin-22. Immunity 39, 372-385.

1-53. (canceled)

54. A method for detecting cancer in a patient comprising detecting a change in IL4I1 expression or enzymatic activity of IL4I1 in a sample obtained from said patient and detecting cancer in said patient if said expression or enzymatic activity of said IL4I1 is increased in said patient when compared to control genes in said sample and wherein the method further comprises detecting the activity or expression of AHR in a sample obtained from said patient, wherein said activity or expression of AHR is modulated when compared to a control.

55. The method according to claim 54, wherein said control genes comprise one or more housekeeping genes.

56. The method according to claim 54, wherein said sample is a control sample from a healthy subject, a group of healthy subjects, a patient or a patient group.

57. The method according to claim 54, wherein said activity or expression of AHR is increased.

58. The method according to claim 54, wherein said detecting enzymatic activity of IL4I1 in said sample comprises the detection of the amount or concentration of IL4I1 metabolites in said sample.

59. The method according to claim 58, wherein said metabolites are selected from metabolites derived from the conversion of IL4I1 of phenylalanine, tyrosine or tryptophan.

60. The method according to claim 59, wherein said metabolites are selected from the group consisting of phenylpyruvic acid (PP), hydroxyphenylpyruvic acid (HPP), indole-3-pyruvic acid (I3P), 2-phenylacetic acid, phenyllactic acid, 4-hydroxybenzaldehyde, 2-hydroxy-2-phenylacetic acid, 4-hydroxyphenyllactic acid, and in particular the I3P derivatives indole-3-acetic acid (IAA), indole-3-aldehyde (I3A) and indole-3-lactic acid (ILA), 4-hydroxyquinoline-2-carboxylic acid (KynA), 1,3-di(1H-indole-3-yl)acetone, (3Z)-1-(1H-indole-3-yl)-3-indole-3-ylidenepropan-2-one, indole-3-carboxylic acid, oxidized indole-3-acetic acid, and indole-3-carbinol and/or the amino acids or amino acid metabolites L-valine, L-isoleucine, L-leucine, L-alanine, L-glutamic acid, L-methionine, L-glutamine, 4-methylsulfanyl-2-oxobutanoate, alpha-keto-isoleucine, alpha-ketoisovalerate, alpha-ketoisocaproic acid, L-proline, and alpha-ketoglutaric acid.

61. The method according to claim 60, wherein said metabolites are combined with ammonia or H₂O₂.

62. The method according to claim 54, wherein detecting the enzymatic activity of IL4I1 comprises the use of chromatography, NMR, metabolite sensors, antibodies, ELISAs, enzymatic assays, colorimetric assays, fluorescence assays or H₂O₂, or ammonia detection.

63. The method according to claim 62, wherein detecting the enzymatic activity of IL4I1 further comprises detecting expression using genetic tools or detecting the amount of protein of IL4I1 using antibodies in biological fluids and cell or tissue samples.

64. A method for detecting increased tumor cell motility in a cancer patient comprising detecting the activity or expression of IL4I1 in a sample obtained from said patient and detecting increased tumor cell motility in said patient if said expression or enzymatic activity of said IL4I1 is increased in said patient when compared to control genes in said sample and wherein said method further comprises detecting the activity or expression of AHR in a sample obtained from said patient, wherein said activity or expression of AHR is modulated when compared to a control.

65. The method according to claim 64, wherein said control genes comprise one or more housekeeping genes.

66. The method according to claim 64, wherein said sample is a control sample from a healthy subject, a group of healthy subjects, a prior sample from the same patient, a different patient or a patient group.

67. The method according to claim 64, wherein said activity or expression of AHR is increased.

68. A method for detecting the effect of cancer immune therapy in a cancer patient comprising detecting the activity or expression of IL4I1 in a sample obtained from said patient, wherein when said expression or enzymatic activity of said IL4I1 is increased in said patient when compared to control genes in said sample a reduced effect of said cancer immune therapy in said patient is indicated and wherein the method further comprises detecting the activity or expression of AHR in a sample obtained from said patient, wherein said activity or expression of AHR is modulated when compared to a control and wherein said increase of said activity or expression of said IL4I1 is detected in response to an immune therapy in said patient.

69. The method of detecting the effect of cancer immune therapy according to claim 68, wherein the cancer immune therapy comprises a combination of immune therapy with IL4I1 modulators, AHR modulators or any other conventional cancer treatments.

70. A method for detecting resistance against a cancer treatment comprising immune therapy in a patient comprising detecting the activity or expression of IL4I1 in a sample obtained from said patient and detecting resistance against a cancer treatment comprising immune therapy in said patient if said expression or enzymatic activity of said IL4I1 is increased in said patient when compared to control genes in said sample and wherein the method further comprises detecting the activity or expression of AHR in a sample obtained from said patient wherein said activity or expression of AHR is modulated when compared to a control.

71. The method for detecting resistance against a cancer treatment comprising immune therapy according to claim 70, wherein the immune therapy comprises immune checkpoint blockade (ICB) or IDO1 inhibitors.

72. The method according to claim 54, wherein said detecting of said activity comprises detecting IL4I1 tryptophan metabolites.

73. The method according to claim 72, wherein said IL4I1 tryptophan metabolites are selected from the group consisting of I3P-derived metabolites, KynA, indole-3-acetic acid (IAA), indole-3-aldehyde (I3A), indole-3-lactic acid (ILA) and indole-3-carbinol.

74. The method according to claim 54, wherein the method for detecting the activity and/or expression of AHR comprises determining an aryl hydrocarbon receptor (AHR) activation signature or AHR nuclear translocation, or the activity of cytochrome P-450 enzymes or the binding of AHR-ARNT to dioxin-responsive elements (DRE) using reporter assays.

75. The method according to claim 54, wherein said biological sample is selected from a the group consisting of biological fluids, mammalian cells, tissues, whole blood, cell lines, cellular supernatants, primary cells, IPSCs, hybridomas, recombinant cells, stem cells, cancer cells, bone cells, cartilage cells, nerve cells, glial cells, epithelial cells, skin cells, scalp cells, lung cells, mucosal cells, muscle cells, skeletal muscles cells, striated muscle cells, smooth muscle cells, heart cells, secretory cells, adipose cells, blood cells, erythrocytes, basophils, eosinophils, monocytes, lymphocytes, T-cells, B-cells, neutrophils, NK cells, regulatory T-cells, dendritic cells, Th17 cells, Th1 cells, Th2 cells, myeloid cells, macrophages, monocyte derived stromal cells, bone marrow cells, spleen cells, thymus cells, pancreatic cells, oocytes, sperm, kidney cells, fibroblasts, intestinal cells, cells of the female or male reproductive tracts, prostate cells, bladder cells, eye cells, corneal cells, retinal cells, sensory cells, keratinocytes, hepatic cells, brain cells, kidney cells, and colon cells, and cancer cells that do not derive from the immune system.

76. The method according to claim 75, wherein said mammalian cells are human cells.

77. The method according to claim 54, wherein said cancer is selected from the group consisting of B cell lymphoid malignancies, follicular lymphoma, Hodgkin lymphoma, primary mediastinal B cell lymphoma, diffuse large B cell lymphoma, marginal zone lymphoma, chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM).

78. The method according to claim 54, further comprising the step of a stratification of said patient to a disease or treatment group.

79. The method according to claim 78, wherein said treatment group receives IL4I1 inhibitors.

80. A diagnostic kit comprising materials for performing a method according to claim 54, in one or separate containers.

81. The diagnostic kit according to claim 80, further comprising auxiliary agents or instructions for performing said method.

82. A method of stratifying cancer patients into a high survival group and a low survival group comprising detecting the activity or expression of IL4I1 in a sample obtained from said cancer patients wherein if said expression or enzymatic activity of said IL4I1 is increased in said patient when compared to control genes in said sample the patient is in the low survival group and wherein if said expression or enzymatic activity of said IL4I1 is unchanged or decreased in said patient when compared to control genes in said sample the patient is in the high survival group.

83. The method of stratifying cancer patients according to claim 82, wherein said increase of said activity or expression of said IL4I1 is detected in response to an immune therapy in said patient.

84. The method according to claim 82, wherein patients in the high survival group are immune therapy responders and patients in the low survival group are immune therapy non-responders.

85. The method according to claim 84, wherein said non-responders are immune checkpoint blockade (ICB) non-responders or IDO1 inhibitors non-responders.

86. A method of stratifying cancer patients into a responders and non-responders group to immune therapy comprising detecting the activity or expression of IL4I1 in a sample obtained from said cancer patients after said patients are subject to immune therapy, wherein if said expression or enzymatic activity of said IL4I1 is increased in said patient when compared to control genes in said sample, the patient is in the non-responders group and wherein if said expression or enzymatic activity of said IL4I1 is unchanged or decreased in said patient when compared to control genes in said sample the patient is in the responders group.

87. The method of stratifying cancer patients into responders and non-responders group to immune therapy according to claim 86 wherein the control sample is from the same patients prior to being subject to said immune therapy.

88. The method of stratifying cancer patients according to claim 86, wherein the method further comprises detecting the activity or expression of AHR in a sample obtained from said patient, wherein said activity or expression of AHR is modulated when compared to a control.

89. The method of stratifying cancer patients according to claim 88, wherein said modulation of said activity or expression of said AHR is detected in response to immune therapy in said patient.

90. The method of stratifying cancer patients according to claim 86, wherein the immune therapy comprises immune checkpoint blockade (ICB) or IDO1 inhibitors.

91. A method of treating a cancer in a subject in need thereof comprising detecting the activity or expression of IL4I1 in a sample obtained from said patient, wherein at least one IL4I1 inhibitor or at least one AHR modulator is administered in an effective amount to said subject if said expression or enzymatic activity of said IL4I1 is increased in said patient when compared to control genes in said sample.

92. The method of treating a cancer according to claim 91, further comprising detecting the activity or expression of AHR in a sample obtained from said patient, wherein said activity or expression of AHR is modulated when compared to a control.

93. The method of treating a cancer according to claim 91, wherein said increase of said activity or expression of said IL4I1 or said modulation of the activity or expression of AHR is detected in response to an immune therapy in said patient.

94. The method of treating a cancer according to claim 91, comprising an immune therapy.

95. The method of treating a cancer according to claim 95, wherein the immune therapy comprises the use of at least one immune checkpoint inhibitor.

96. The method of treating a cancer according to claim 95, further comprising an anti-CTLA4, anti-PD-L1, anti-PD1, anti-TIM3, anti-TIGIT, anti-LAG3 or a combination thereof.

97. The method of treating a cancer according to claim 92, wherein the immune therapy comprises a combination of immune therapy with inhibitors of IDO1, inhibitors of TDO2, AHR modulators and any other cancer treatments.

98. The method of treating a cancer according to claim 91, wherein the IL4I1 inhibitor is selected from the group consisting of 3-phenyl-2-piperidin-1-ylpropanoic acid, 2-(4-methylpiperazin-1-yl)-3-phenylpropanoic acid, 2-(diethylamino)-3-phenylpropanoic acid, 3-(2,6-dichlorophenyl)-2-piperidin-1-ylpropanoic acid, 3-phenyl-2-(propylamino)propanoic acid, 2-anilino-3-phenylpropanoic acid, L-Phenylalanine N-2-propen-1-yl-3-(trifluoromethyl), L-Phenylalanine, 4-cyano-N-phenyl, N-Propyl-L-phenylalanine, N-Phenyl-L-phenylalanine, 2-amino-3-phenyl-propionic acid ethyl ester, 2-acteylamino-3-phenyl-propionic acid and 3-(2-pyridyl)-alanine.

99. The method of treating a cancer according to claim 91, wherein the modulator of AHR is selected from the group consisting of a 2-phenylpyrimidine-4-carboxamide compound, a sulphur substituted 3-oxo-2,3-dihydropyridazine-4-carboxamide compound, a 3-oxo-6-heteroaryl-2-phenyl-2,3-dihydropyridazine-4-carboxamide compound, a 2-hetarylpyrimidine-4-carboxamide compound, a 3-oxo-2,6-diphenyl-2,3-dihydropyridazine-4-carboxamide compound, a 2-heteroaryl-3-oxo-2,3-dihydro-4-carboxamide compound, PDM 2, 1,3-dichloro-5-[(1E)-2-(4-methoxyphenyl)ethenyl]-benzene, α-Naphthoflavone, 6, 2′,4′-Trimethoxyflavone, CH223191, a tetrahydropyridopyrimidine derivative, StemRegenin-1, CH223191, GNF351, CB 7993113 HP163, PX-A590, PX-A548, PX-A275, PX-A758, PX-A446, PX-A24590, PX-A25548, PX-A25275, PX-A25758, PX-A26446, an Indole AHR inhibitor, and an oxazole-containing (OxC) compound.

100. The method of treating a cancer according to claim 91, wherein said cancer is selected from the group consisting of B cell lymphoid malignancies, follicular lymphoma, Hodgkin lymphoma, primary mediastinal B cell lymphoma, diffuse large B cell lymphoma, marginal zone lymphoma and chronic lymphoid leukemia, Adrenocortical carcinoma (ACC), Bladder Urothelial Carcinoma (BLCA), Breast invasive carcinoma (BRCA), Cervical squamous cell carcinoma and endocervical adenocarcinoma (CESC), Cholangiocarcinoma (CHOL), Colon adenocarcinoma (COAD), Lymphoid Neoplasm Diffuse Large B-cell Lymphoma (DLBC), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Brain Lower Grade Glioma (LGG), Liver hepatocellular carcinoma (LIHC), Lung adenocarcinoma (LUAD), Lung squamous cell carcinoma (LUSC), Mesothelioma (MESO), Ovarian serous cystadenocarcinoma (OV), Pancreatic adenocarcinoma (PAAD), Pheochromocytoma and Paraganglioma (PCPG), Prostate adenocarcinoma (PRAD), Rectum adenocarcinoma (READ), Sarcoma (SARC), Skin Cutaneous Melanoma (SKCM), Stomach adenocarcinoma (STAD), Testicular Germ Cell Tumors (TGCT), Thyroid carcinoma (THCA), Thymoma (THYM), Uterine Corpus Endometrial Carcinoma (UCEC), Uterine Carcinosarcoma (UCS), and Uveal Melanoma (UVM).
